(19) United States
(12) Patent Application Publication
(10) Pub. No.: US 2026/0240861 A1
(43) Pub. Date: Aug. 20, 2026

(54) CDK8/19 DUAL INHIBITORS AND METHODS OF USE THEREOF

(71) Applicant: Insilico Medicine IP Limited, Hong Kong (CN)

(72) Inventors: Xiao DING, Shanghai (CN); Xiaosong LIU, Shanghai (CN); Feng REN, Shanghai (CN); Jianyu XU, Shanghai (CN)

(21) Appl. No.: 19/161,057

(22) PCT Filed: Mar. 5, 2024
(86) PCT No.: PCT/CN2024/080162

§ 371 Date: Aug. 29, 2025

(30) Foreign Application Priority Data
Mar. 6, 2023 (WO) PCT/CN2023/079857
Dec. 13, 2023 (WO) PCT/CN2023/138484

(51) Int. Cl. A61K 31/5377; A61K 31/4184; A61K 31/438; A61K 31/439; A61K 31/4439; A61K 31/454; A61K 31/496; A61K 31/4995; A61K 31/551; C07B 59/00; C07D 405/04; C07D 405/14; C07D 409/04; C07D 413/14; C07D 471/04; C07D 471/08; C07D 487/04; C07D 487/08

(52) U.S. Cl. CPC A61K 31/5377; A61K 31/4184; A61K 31/438; A61K 31/439; A61K 31/4439; A61K 31/454; A61K 31/496; A61K 31/4995; A61K 31/551; C07B 59/002; C07D 405/04; C07D 405/14; C07D 409/04; C07D 413/14; C07D 471/04; C07D 471/08; C07D 487/04; C07D 487/08; C07B 2200/05

(57) ABSTRACT

The disclosure provides for compounds and methods for modulating or inhibiting CDK8/19.

### CROSS-REFERENCE

This patent application claims the benefit of International Application No. PCT/CN2023/079857, filed Mar. 6, 2023 and International Application No. PCT/CN2023/138484, filed Dec. 13, 2023; and which are incorporated herein by reference in their entirety.

### FIELD

The disclosure relates to compounds that function as dual inhibitors of cyclic dependent kinase 8 and/or 19 (CDK8/19) and methods of use.

### BACKGROUND

Cyclin-dependent kinase 8 (“CDK8”) and its paralog, CDK19, collectively termed “Mediator Kinase”, are cyclin-dependent kinases that have been implicated as key rheostats in cellular homeostasis and developmental programming. CDK8 and CDK19 are incorporated, in a mutually exclusive manner, as part of a 4-protein complex called the Mediator kinase module. This module reversibly associates with the Mediator, a 26 subunit protein complex that regulates RNA Polymerase II mediated gene expression. As part of this complex, the Mediator kinases have been implicated in diverse process such as developmental signaling, metabolic homeostasis and in innate immunity. In recent years, dysregulation of Mediator kinase module proteins, including CDK8/19, has been implicated in the development of different human diseases, and in particular for oncological purposes.

### SUMMARY

The present disclosure addresses the above need and provides additional advantages as well.

Disclosed herein is a compound of Formula (Ia), or a pharmaceutically acceptable salt or stereoisomer thereof:

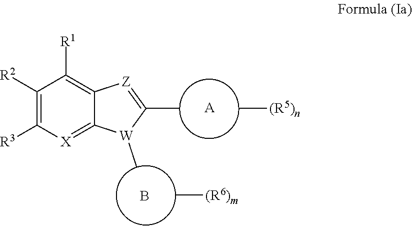

as disclosed herein.

Also disclosed herein is a pharmaceutical composition comprising a compound disclosed herein, or a pharmaceutically acceptable salt or stereoisomer thereof, and at least one pharmaceutically acceptable excipient.

A method of inhibiting cyclic dependent kinase 8 and/or 19 (CDK8/19) activity in a subject, comprising administering to the subject a compound disclosed herein, or a pharmaceutically acceptable salt or stereoisomer thereof, or a pharmaceutical composition disclosed herein.

A method of treating cancer in a subject in need thereof, the method comprising administering a therapeutically effective amount of a compound disclosed herein, or a pharmaceutically acceptable salt or stereoisomer thereof, or a pharmaceutical composition disclosed herein.

In some embodiments, the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM). In some embodiments, the cancer is AML.

In some embodiments, the cancer is a solid cancer. In some embodiments, the cancer is a prostate cancer, breast cancer, colon cancer, pancreatic cancer, skin cancer, ocular cancer, gastrointestinal cancer, thyroid cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, head and neck squamous cell carcinomas, melanoma. A method of treating an auto-immune disease or disorder in a subject in need thereof, the method comprising administering a therapeutically effective amount of a compound disclosed herein, or a pharmaceutically acceptable salt or stereoisomer thereof, or a pharmaceutical composition disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Carboxyl” refers to —COOH.

“Cyano” refers to —CN.

“Alkyl” refers to a straight-chain, or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to a straight-chain, or branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH—CH₂), isopropenyl[—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight-chain or branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkylene is optionally substituted with oxo, halogen, —CN, —COOH, COOMe, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula-Oalkyl where alkyl is as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising 6 to 30 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic, or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ fully saturated cycloalkyl or C₃-C₁₅ cycloalkenyl), from three to ten carbon atoms (C₃-C₁₀ fully saturated cycloalkyl or C₃-C₁₀ cycloalkenyl), from three to eight carbon atoms (C₃-C₈ fully saturated cycloalkyl or C₃-C₈ cycloalkenyl), from three to six carbon atoms (C₃-C₆ fully saturated cycloalkyl or C₃-C₆ cycloalkenyl), from three to five carbon atoms (C₃-C₅ fully saturated cycloalkyl or C₃-C₈ cycloalkenyl), or three to four carbon atoms (C₃-C₄ fully saturated cycloalkyl or C₃-C₄ cycloalkenyl). In some embodiments, the cycloalkyl is a 3- to 10-membered fully saturated cycloalkyl or a 3- to 10-membered cycloalkenyl. In some embodiments, the cycloalkyl is a 3- to 6-membered fully saturated cycloalkyl or a 3- to 6-membered cycloalkenyl. In some embodiments, the cycloalkyl is a 5- to 6-membered fully saturated cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g., —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ fully saturated heterocycloalkyl or C₂-C₁₀ heterocycloalkenyl), from two to eight carbon atoms (C₂-C₈ fully saturated heterocycloalkyl or C₂-C₈ heterocycloalkenyl), from two to seven carbon atoms (C₂-C₇ fully saturated heterocycloalkyl or C₂-C₇ heterocycloalkenyl), from two to six carbon atoms (C₂-C₆ fully saturated heterocycloalkyl or C₂-C₆ heterocycloalkenyl), from two to five carbon atoms (C₂-C₅ fully saturated heterocycloalkyl or C₂-C₈ heterocycloalkenyl), or two to four carbon atoms (C₂-C₄ fully saturated heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns that are sterically impractical and/or synthetically non-feasible. Thus, any substituents described should generally be understood as having a maximum molecular weight of about 1,000 daltons, and more typically, up to about 500 daltons.

The term “one or more” when referring to an optional substituent means that the subject group is optionally substituted with one, two, three, four, or more substituents. In some embodiments, the subject group is optionally substituted with one, two, three or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

An “effective amount” or “therapeutically effective amount” refers to an amount of a compound administered to a mammalian subject, either as a single dose or as part of a series of doses, which is effective to produce a desired therapeutic effect.

The terms “treat,” “treating,” or “treatment,” as used herein, include alleviating, abating, or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

As used herein, a “disease or disorder associated with cyclic dependent kinase 8 and/or 19 (CDK8/19)” or, alternatively, “a cyclic dependent kinase 8 and/or 19 (CDK8/19)-mediated disease or disorder” means any disease or other deleterious condition in which cyclic dependent kinase 8 and/or 19 (CDK8/19), or a mutant thereof, is known or suspected to play a role.

### Compounds

Described herein are compounds, or a pharmaceutically acceptable salt or stereoisomer thereof useful in the treatment of a disease or disorder associated with cyclic dependent kinase 8 and/or cyclic dependent kinase 19.

Disclosed herein is a compound of Formula (I), or a pharmaceutically acceptable salt or stereoisomer thereof:

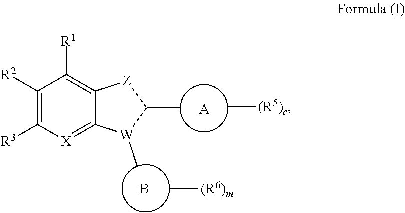

wherein:
R¹ is hydrogen, halogen, —CN, NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
R² is fluoro, chloro, or bromo and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or
R³ is fluoro, chloro, or bromo and R² is hydrogen, fluoro, chloro, iodo, —CN, —OH, —OR^{a}, —S(═O) R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
X is N or CR⁴;
R⁴ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;

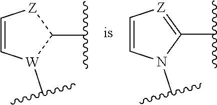

and Z is —N— or —CR^{Z1}—;
R^{Z1} is hydrogen, halogen, —SF⁵, —CN, —NO₂, —OH, —OR^{a}, —NR^{c}R^{d}, —C(═O)R^{a}, —C(═O)OR⁵, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;

or

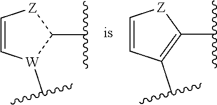

and Z is —NR^{Z2}—, —O—, or —S—;
R^{Z2} is hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
Ring A is cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SF⁵, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or two R⁵ on the same atom are taken together to form an oxo;
each R^{5a} is independently halogen, —CN, —NO₂, —OH, —OR^{a}, —OC(═O)R^{a}, —OC(═O)OR⁵, —OC(═O)NR^{c}R^{d}, —SF⁵, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NRDC(═O)NR^{c}R^{d}, —NRDC(═O)R^{a}, —NRDC(═O)OR^{b}, —NR^{b}S(═O)₂R^{a}, —N═S(═O)(R^{b})₂, —C(═O) R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
n is 0, 1, 2, 3, 4, 5, or 6;
Ring B is cycloalkyl, heterocycloalkyl, aryl, or heteroaryl;
each R⁶ is independently halogen, —CN, —NO₂, —OH, —OR^{a}, —OC(═O) R^{a}, —OC(═O)OR^{b}, —OC(═O)NR^{c}R^{d}, —SF⁵, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{6a};
or two R⁶ on the same atom are taken together to form an oxo;
or two R⁶ on the same atom are taken together to form ═NH;
each R^{6a} is independently halogen, —CN, —NO₂, —OH, —OR^{a}, —OC(═O)R^{a}, —OC(═O)OR^{b}, —OC(═O)NR^{c}R^{d}, —SF⁵, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{6a} on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
L is absent or C₁-C₃ alkylene optionally substituted with one or more R;
each R is independently halogen, —CN, —OH, —SF⁵, —SH, —S(═O) C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O) C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl.

In some embodiments, the compound of Formula (I), or a pharmaceutically acceptable salt or stereoisomer thereof, is a CDK8 inhibitor.

In some embodiments, the compound of Formula (I), or a pharmaceutically acceptable salt or stereoisomer thereof,
R² is bromo and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R; or
R³ is bromo and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R.

In some embodiments, the compound of Formula (I), or a pharmaceutically acceptable salt or stereoisomer thereof, R² is bromo and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or
R³ is bromo and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
X is CR⁴.

In some embodiments of a compound of Formula (I), the compound is of Formula (Ia):

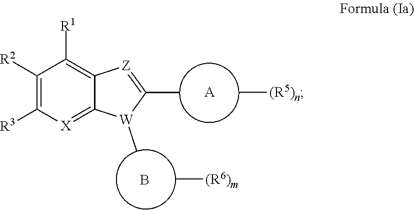

wherein W is —N—.

In some embodiments of a compound of Formula (I), the compound is of Formula (Ib):

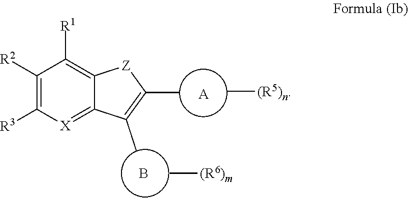

In some embodiments of a compound of Formula (I), the compound is of Formula (Ic-1) or (Ic-2):

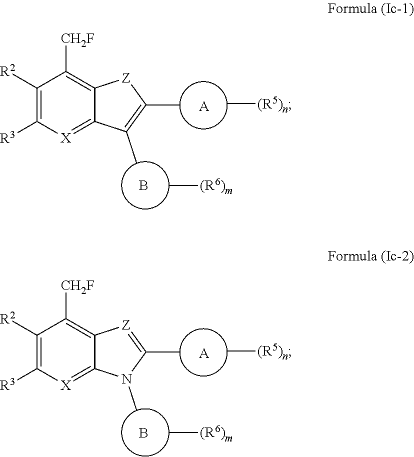

In some embodiments of a compound of Formula (I), the compound is of Formula (Id-1) or (Id-2):

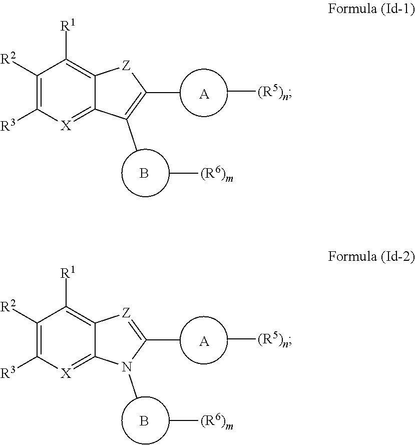

In some embodiments of a compound of Formula (I), the compound is of Formula (Id-3) or (Id-4):

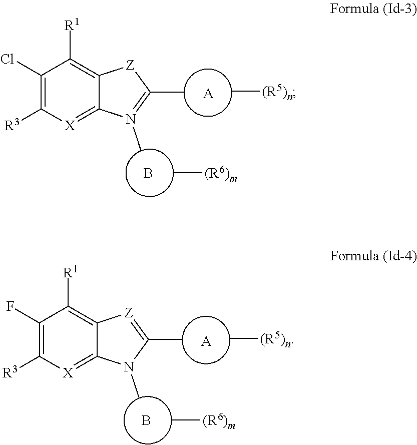

In some embodiments of a compound of Formula (I), the compound is of Formula (Ie-1) or (Ie-2):

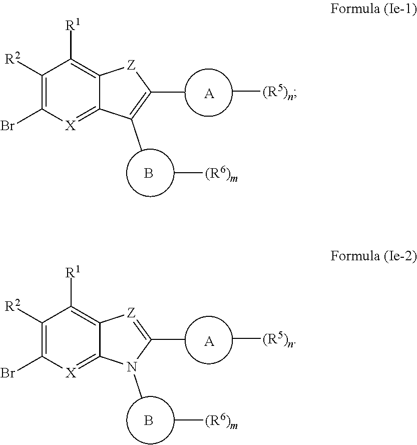

In some embodiments of a compound of Formula (I), (Ib), (Ic-1), (Id-1), or (Ie-1), Z is —NR^{Z2}—. In some embodiments, Z is —O—. In some embodiments, Z is —S—.

In some embodiments of a compound of Formula (I), (Ia), (Ib), (Ic-1), (Id-1), or (Ie-1), R^{Z2} is hydrogen, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, R^{Z2} is hydrogen, C₁-C₆alkyl, or C₁-C₆haloalkyl. In some embodiments, R^{Z2} is hydrogen or C₁-C₆alkyl. In some embodiments, R^{Z2} is hydrogen. In some embodiments, R^{Z2} is C₁-C₆alkyl.

In some embodiments of a compound of Formula (I), (Ia), (Ic-2), (Id-2), (Id-3), (Id-4), or (Ic-2), Z is —N—. In some embodiments, Z is —CR^{Z1}—.

In some embodiments of a compound of Formula (I), (Ia), (Ic-2), (Id-2), (Id-3), (Id-4), or (Ie-2), R^{Z1} is hydrogen, halogen, —SF⁵, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, R^{Z1} is hydrogen, halogen, —CN, C₁-C₆alkyl, C₁-C₆haloalkyl, C₁-C₆hydroxyalkyl, C₁-C₆aminoalkyl, or C₁-C₆heteroalkyl. In some embodiments, R^{Z1} is hydrogen, halogen, —CN, C₁-C₆alkyl, or C₁-C₆haloalkyl. In some embodiments, R^{Z1} is hydrogen, halogen, —CN, or C₁-C₆alkyl. In some embodiments, R^{Z1} is hydrogen or —CN. In some embodiments, R^{Z1} is hydrogen. In some embodiments, R^{Z1} is —CN.

In some embodiments of a compound of Formula (I), (Ia), (Ib), (Ic-1), or (Ic-2), R² is bromo and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R² is bromo and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R² is bromo and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiment, R² is bromo and R³ is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R² is bromo and R³ is hydrogen, fluoro, chloro, iodo, —CN, —OH, —OR^{a}, C₁-C₆alkyl, C₁-C₆haloalkyl, cycloalkyl, or heterocycloalkyl. In some embodiments, R² is bromo and R³ is hydrogen, fluoro, chloro, iodo, —CN, —OH, —OR^{a}, C₁-C₆alkyl, or C₁-C₆haloalkyl. In some embodiments, R² is bromo and R³ is hydrogen, fluoro, chloro, C₁-C₆alkyl, or C₁-C₆haloalkyl. In some embodiments, R² is bromo and R³ is hydrogen.

In some embodiments of a compound of Formula (I), (Ia), (Ib), (Ic-1), or (Ic-2), R² is fluoro and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R² is fluoro and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R² is fluoro and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R² is fluoro and R³ is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R² is fluoro and R³ is hydrogen, fluoro, chloro, iodo, —CN, —OH, —OR^{a}, C₁-C₆alkyl, C₁-C₆haloalkyl, cycloalkyl, or heterocycloalkyl. In some embodiments, R² is fluoro and R³ is hydrogen, fluoro, chloro, iodo, —CN, —OH, —OR^{a}, C₁-C₆alkyl, or C₁-C₆haloalkyl. In some embodiments, R² is fluoro and R³ is hydrogen, fluoro, chloro, C₁-C₆alkyl, or C₁-C₆haloalkyl. In some embodiments, R² is fluoro and R³ is hydrogen.

In some embodiments of a compound of Formula (I), (Ia), (Ib), (Ic-1), or (Ic-2), R² is chloro and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R² is chloro and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R² is chloro and R³ is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R² is chloro and R³ is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R² is chloro and R³ is hydrogen, fluoro, chloro, iodo, —CN, —OH, —OR^{a}, C₁-C₆alkyl, C₁-C₆haloalkyl, cycloalkyl, or heterocycloalkyl. In some embodiments, R² is chloro and R³ is hydrogen, fluoro, chloro, iodo, —CN, —OH, —OR^{a}, C₁-C₆alkyl, or C₁-C₆haloalkyl. In some embodiments, R² is chloro and R³ is hydrogen, fluoro, chloro, C₁-C₆alkyl, or C₁-C₆haloalkyl. In some embodiments, R² is chloro and R³ is hydrogen.

In some embodiments of a compound of Formula (I), (Ia), (Ib), (Ic-1), or (Ic-2), R³ is bromo and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R³ is bromo and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R³ is bromo and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R³ is bromo and R² is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R³ is bromo and R² is hydrogen, fluoro, chloro, iodo, —CN, —OH, —OR^{a}, C₁-C₆alkyl, C₁-C₆haloalkyl, cycloalkyl, or heterocycloalkyl. In some embodiments, R³ is bromo and R² is hydrogen, fluoro, chloro, iodo, —CN, —OH, —OR^{a}, C₁-C₆alkyl, or C₁-C₆haloalkyl. In some embodiments, R³ is bromo and R² is hydrogen, fluoro, chloro, C₁-C₆alkyl, or C₁-C₆haloalkyl. In some embodiments, R³ is bromo and R² is hydrogen or chloro. In some embodiments, R³ is bromo and R² is hydrogen.

In some embodiments of a compound of Formula (I), (Ia), (Ib), (Ic-1), or (Ic-2), R³ is fluoro and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O) R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R³ is fluoro and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R³ is fluoro and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R³ is fluoro and R² is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R³ is fluoro and R² is hydrogen, fluoro, chloro, iodo, —CN, —OH, —OR^{a}, C₁-C₆alkyl, C₁-C₆haloalkyl, cycloalkyl, or heterocycloalkyl. In some embodiments, R³ is fluoro and R² is hydrogen, fluoro, chloro, iodo, —CN, —OH, —OR^{a}, C₁-C₆alkyl, or C₁-C₆haloalkyl. In some embodiments, R³ is fluoro and R² is hydrogen, fluoro, chloro, C₁-C₆alkyl, or C₁-C₆haloalkyl. In some embodiments, R³ is fluoro and R² is hydrogen or chloro. In some embodiments, R³ is fluoro and R² is hydrogen.

In some embodiments of a compound of Formula (I), (Ia), (Ib), (Ic-1), or (Ic-2), R³ is chloro and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R³ is chloro and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, heterocycloalkyl, aryl, or heteroaryl; wherein each alkyl, cycloalkyl, heterocycloalkyl, aryl, and heteroaryl is independently optionally substituted with one or more R. In some embodiments, R³ is chloro and R² is hydrogen, fluoro, chloro, iodo, —CN, —OH, —OR^{a}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R³ is chloro and R² is hydrogen, fluoro, chloro, iodo, —CN, —OH, —OR^{a}, C₁-C₆alkyl, C₁-C₆haloalkyl, cycloalkyl, or heterocycloalkyl; wherein each alkyl, cycloalkyl, and heterocycloalkyl is independently optionally substituted with one or more R. In some embodiments, R³ is chloro and R² is hydrogen, fluoro, chloro, iodo, —CN, —OH, —OR^{a}, C₁-C₆alkyl, C₁-C₆haloalkyl, cycloalkyl, or heterocycloalkyl. In some embodiments, R³ is chloro and R² is hydrogen, fluoro, chloro, iodo, —CN, —OH, —OR^{a}, C₁-C₆alkyl, or C₁-C₆haloalkyl. In some embodiments, R³ is chloro and R² is hydrogen, fluoro, chloro, C₁-C₆alkyl, or C₁-C₆haloalkyl. In some embodiments, R³ is chloro and R² is hydrogen or chloro. In some embodiments, R³ is chloro and R² is hydrogen.

In some embodiments of a compound of Formula (Id-1), (Id-2), (Id-3), or (Id-4), R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃₋₁₀ cycloalkyl, 3-10 membered heterocycloalkyl, C₆₋₁₀ aryl, or 3-10 membered heteroaryl; wherein each alkyl, alkenyl, alkynyl, cycloalkyl, heterocycloalkyl, aryl, and heteroaryl is independently optionally substituted with one or more R. In some embodiments, R³ is hydrogen, fluoro, chloro, iodo, —CN, —OH, —OR^{a}, C₁-C₃alkyl, C₁-C₃haloalkyl, C₁-C₃hydroxyalkyl, C₁-C₃aminoalkyl, C₁-Czheteroalkyl, 5-6 membered cycloalkyl, 5-6 membered heterocycloalkyl, phenyl, or 5-6 membered heteroaryl; wherein each alkyl, cycloalkyl, heterocycloalkyl, phenyl, and heteroaryl is independently optionally substituted with one or more R. In some embodiments, R³ is hydrogen, fluoro, chloro, iodo, —CN, —OH.

In some embodiments of a compound of Formula (Ie-1) or (Ie-2), R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, C₃₋₁₀ cycloalkyl, 3-10 membered heterocycloalkyl, C₆₋₁₀ aryl, or 3-10 membered heteroaryl; wherein each alkyl, alkenyl, alkynyl, cycloalkyl, heterocycloalkyl, aryl, and heteroaryl is independently optionally substituted with one or more R. In some embodiments, R² is hydrogen, fluoro, chloro, iodo, —CN, —OH, —OR^{a}, C₁-C₃alkyl, C₁-C₃haloalkyl, C₁-C₃hydroxyalkyl, C₁-C₃aminoalkyl, C₁-C₃heteroalkyl, 5-6 membered cycloalkyl, 5-6 membered heterocycloalkyl, phenyl, or 5-6 membered heteroaryl; wherein each alkyl, cycloalkyl, heterocycloalkyl, phenyl, and heteroaryl is independently optionally substituted with one or more R. In some embodiments, R² is hydrogen, fluoro, chloro, iodo, —CN, —OH.

In some embodiments of a compound of Formula (I), (Ia), (Ib), (Id-1), (Id-2), (Id-3), (Id-4), (Ie-1), or (Ie-2), R¹ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R¹ is halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R. In some embodiments, R¹ is halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R¹ is halogen, C₁-C₆alkyl, or C₁-C₆haloalkyl; wherein each alkyl is independently optionally substituted with one or more R. In some embodiments, R¹ is halogen, C₁-C₆alkyl, or C₁-C₆haloalkyl. In some embodiments, R¹ is C₁-C₆alkyl or C₁-C₆haloalkyl. In some embodiments, R¹ is halogen or C₁-C₆haloalkyl. In some embodiments, R¹ is C₁-C₆haloalkyl. In some embodiments, R¹ is C₁-C₃haloalkyl. In some embodiments, R¹ is-OH, —NO₂, —CN, —CH₂OH, —CF₂H, —CH₂CH₂OH, —CF₃, —CH₃, —CH₂CH₃, —CH(CH₃)₂, —CH═CH₂, ethynyl, cyclopropyl, cyclobutyl, or imidazolyl. In some embodiments, R¹ is CF₂H.

In some embodiments of a compound of Formula (I), (Ia), (Ib), (Ic-1), (Ic-2), (Id-1), (Id-2), (Id-3), (Id-4), (Ie-1), or (Ie-2), X is N. In some embodiments, X is CR⁴.

In some embodiments of a compound of Formula (I), (Ia), (Ib), (Ic-1), (Ic-2), (Id-1), (Id-2), (Id-3), (Id-4), (Ie-1), or (Ie-2), R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R. In some embodiments, R⁴ is hydrogen, halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, R⁴ is hydrogen, halogen, C₁-C₆alkyl, C₁-C₆haloalkyl, cycloalkyl, or heterocycloalkyl. In some embodiments, R⁴ is hydrogen, halogen, C₁-C₆alkyl, or C₁-C₆haloalkyl. In some embodiments, R⁴ is hydrogen, halogen, or C₁-C₆alkyl. In some embodiments, R⁴ is hydrogen or halogen. In some embodiments, R⁴ is halogen. In some embodiments, R⁴ is hydrogen.

In some embodiments of a compound of Formula (I), (Ia), (Ib), (Ic-1), (Ic-2), (Id-1), (Id-2), (Id-3), (Id-4), (Ie-1), or (Ie-2), Ring A is cycloalkyl or heterocycloalkyl.

In some embodiments of a compound of Formula (I), (Ia), (Ib), (Ic-1), (Ic-2), (Id-1), (Id-2), (Id-3), (Id-4), (Ie-1), or (Ie-2), Ring A is cycloalkyl.

In some embodiments of a compound of Formula (I), (Ia), (Ib), (Ic-1), (Ic-2), (Id-1), (Id-2), (Id-3), (Id-4), (Ie-1), or (Ie-2), Ring A is a monocyclic or bicyclic cycloalkyl. In some embodiments, Ring A is a monocyclic cycloalkyl. In some embodiments, Ring A is a monocyclic 3- to 9-membered cycloalkyl. In some embodiments, Ring A is a monocyclic 3- to 7-membered cycloalkyl. In some embodiments, Ring A is a monocyclic 5- to 7-membered cycloalkyl. In some embodiments, Ring A is a monocyclic 5- to 6-membered cycloalkyl. In some embodiments, Ring A is a bicyclic cycloalkyl. In some embodiments, Ring A is a bicyclic 7- to 12-membered cycloalkyl. In some embodiments, Ring A is a fused or spiro bicyclic 7- to 12-membered cycloalkyl. In some embodiments, Ring A is a spiro bicyclic 7- to 12-membered cycloalkyl. In some embodiments, Ring A is a fused bicyclic 7- to 12-membered cycloalkyl.

In some embodiments of a compound of Formula (I), (Ia), (Ib), (Ic-1), (Ic-2), (Id-1), (Id-2), (Id-3), (Id-4), (Ie-1), or (Ie-2), Ring A is heterocycloalkyl.

In some embodiments of a compound of Formula (I), (Ia), (Ib), (Ic-1), (Ic-2), (Id-1), (Id-2), (Id-3), (Id-4), (Ie-1), or (Ie-2), Ring A is a monocyclic or bicyclic 3- to 10-membered heterocycloalkyl comprising one, two, or three heteroatoms selected from the group consisting of N, O, S, and P. In some embodiments, Ring A is a monocyclic or bicyclic 3- to 10-membered heterocycloalkyl comprising one, two, or three heteroatoms selected from the group consisting of N, O, and S. In some embodiments, Ring A is a monocyclic or bicyclic 3- to 10-membered heterocycloalkyl comprising one, two, or three heteroatoms selected from the group consisting of N and O. In some embodiments, Ring A is a monocyclic or bicyclic 3- to 10-membered heterocycloalkyl comprising one or two heteroatoms selected from the group consisting of N, O, S, and P. In some embodiments, Ring A is a monocyclic or bicyclic 3- to 10-membered heterocycloalkyl comprising one or two heteroatoms selected from the group consisting of N, O, and S. In some embodiments, Ring A is a monocyclic or bicyclic 3- to 10-membered heterocycloalkyl comprising one or two heteroatoms selected from the group consisting of N and O. In some embodiments, Ring A is a monocyclic or bicyclic 3- to 10-membered heterocycloalkyl comprising one or two heteroatoms that are N.

In some embodiments of a compound of Formula (I), (Ia), (Ib), (Ic-1), (Ic-2), (Id-1), (Id-2), (Id-3), (Id-4), (Ie-1), or (Ie-2), Ring A is a monocyclic 5- to 7-membered heterocycloalkyl comprising one or two heteroatoms selected from the group consisting of N, O, S, and P. In some embodiments, Ring A is a monocyclic 5- to 7-membered heterocycloalkyl comprising one or two heteroatoms selected from the group consisting of N, O, and S. In some embodiments, Ring A is a monocyclic 5- to 6-membered heterocycloalkyl comprising one or two heteroatoms selected from the group consisting of N, O, and S. In some embodiments, Ring A is a monocyclic 5- to 7-membered heterocycloalkyl comprising one or two heteroatoms selected from the group consisting of N and O. In some embodiments, Ring A is a monocyclic 5- to 7-membered heterocycloalkyl comprising one or two heteroatoms that are N. In some embodiments, Ring A is pyrrolidinyl, piperidinyl, piperazinyl, or morpholinyl. In some embodiments, Ring A is pyrrolidinyl, piperidinyl, or piperazinyl. In some embodiments, Ring A is pyrrolidinyl. In some embodiments, Ring A is piperidinyl or piperazinyl. In some embodiments, Ring A is piperidinyl. In some embodiments, Ring A is piperazinyl. In some embodiments, Ring A is morpholinyl. In some embodiments, Ring A is

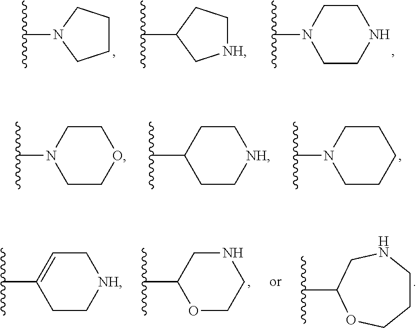

In some embodiments, Ring A is

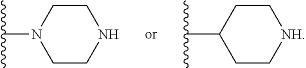 [SMILES: *N1CCNCC1 ; *C1CCNCC1]

In some embodiments, Ring A is

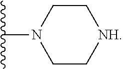 [SMILES: *N1CCNCC1]

In some embodiments, Ring A is

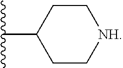 [SMILES: *C1CCNCC1]

In some embodiments of a compound of Formula (I), (Ia), (Ib), (Ic-1), (Ic-2), (Id-1), (Id-2), (Id-3), (Id-4), (Ie-1), or (Ie-2), Ring A is a bicyclic heterocycloalkyl. In some embodiments, Ring A is a fused bicyclic heterocycloalkyl. In some embodiments, Ring A is a spiro bicyclic heterocycloalkyl. In some embodiments, Ring A is a bridged bicyclic heterocycloalkyl. In some embodiments, Ring A is a bicyclic 6- to 12-membered heterocycloalkyl. In some embodiments, Ring A is a bicyclic 6-, 7-, 8-, 9-, 10-, 11-, or 12-membered heterocycloalkyl. In some embodiments, Ring A is a bicyclic 6- to 12-membered heterocycloalkyl comprising one, two, or three heteroatoms selected from the group consisting of N, O, S, and P. In some embodiments, Ring A is a bicyclic 7- to 12-(e.g., 7- to 11-, 7- to 10-, 7- to 9-, 7- to 8-, 8- to 12-, 8- to 11-, 8- to 10-, 8- to 9-, 9- to 12-, 9- to 11-, or 9- to 10-) membered heterocycloalkyl comprising one, two, or three heteroatoms selected from the group consisting of N, O, S, and P. In some embodiments, Ring A is a bicyclic 6- to 10-(e.g., 6- to 9-, 6- to 8-, 6- to 7-, 7- to 10-, 7- to 9-, 7- to 8-, 8- to 10-, 8- to 9-, or 9- to 10-) membered heterocycloalkyl comprising one, two, or three heteroatoms selected from the group consisting of N, O, S, and P. In some embodiments, Ring A is a bicyclic 6- to 10-(e.g., 6- to 9-, 6- to 8-, 6- to 7-, 7- to 10-, 7- to 9-, 7- to 8-, 8- to 10-, 8- to 9-, or 9- to 10-) membered heterocycloalkyl comprising one, two, or three heteroatoms selected from the group consisting of N, O, and S. In some embodiments, Ring A is a bicyclic 6- to 10-(e.g., 6- to 9-, 6- to 8-, 6- to 7-, 7- to 10-, 7- to 9-, 7- to 8-, 8- to 10-, 8- to 9-, or 9- to 10-) membered heterocycloalkyl comprising one, two, or three heteroatoms selected from the group consisting of N and O. In some embodiments, Ring A is a bicyclic 6- to 10-(e.g., 6- to 9-, 6- to 8-, 6- to 7-, 7- to 10-, 7- to 9-, 7- to 8-, 8- to 10-, 8- to 9-, or 9- to 10-) membered heterocycloalkyl comprising one or two heteroatoms selected from the group consisting of N, O, and S. In some embodiments, Ring A is a bicyclic 6- to 10-(e.g., 6- to 9-, 6- to 8-, 6- to 7-, 7- to 10-, 7- to 9-, 7- to 8-, 8- to 10-, 8- to 9-, or 9- to 10-) membered heterocycloalkyl comprising one or two heteroatoms selected from the group consisting of N and O. In some embodiments, Ring A is a bicyclic 6- to 10-membered heterocycloalkyl comprising one or two heteroatoms that are N. In some embodiments, Ring A is

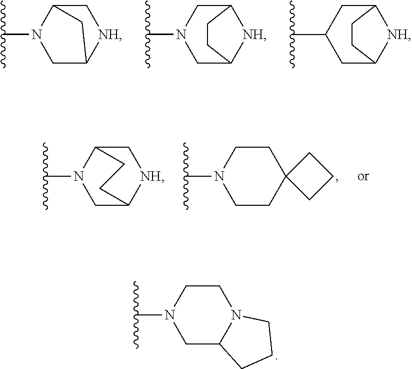

In some embodiments of a compound of Formula (I), (Ia), (Ib), (Ic-1), (Ic-2), (Id-1), (Id-2), (Id-3), (Id-4), (Ie-1), or (Ie-2), Ring A is heteroaryl. In some embodiments, Ring A is 5- to 12-membered heteroaryl comprising one, two, or three heteroatoms selected from the group consisting of N, O, S, and P. In some embodiments, Ring A is 5- to 6-membered heteroaryl comprising one, two, or three heteroatoms selected from the group consisting of N, O, S, and P. In some embodiments, Ring A is 5- to 6-membered heteroaryl comprising one, two, or three heteroatoms selected from the group consisting of N, O, and S. In some embodiments, Ring A is monocyclic 5- to 6-membered heteroaryl comprising one, two, or three heteroatoms selected from the group consisting of N. In some embodiments, Ring A is

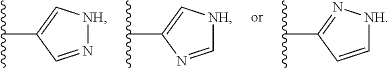

In some embodiments of a compound of Formula (I), (Ia), (Ib), (Ic-1), (Ic-2), (Id-1), (Id-2), (Id-3), (Id-4), (Ie-1), or (Ie-2), each R⁵ is independently halogen, —CN, —OH, —OR^{a}, —NR^{c}R^{d}, —C(═O) R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein each alkyl, alkenyl, alkynyl, cycloalkyl, and heterocycloalkyl is independently optionally substituted with one or more R^{5a}; or two R⁵ on the same atom are taken together to form an oxo. In some embodiments, each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, or heterocycloalkyl; wherein each alkyl, alkenyl, alkynyl, cycloalkyl, and heterocycloalkyl is independently optionally substituted with one or more R^{5a}; or two R⁵ on the same atom are taken together to form an oxo. In some embodiments, each R⁵ is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, or C₂-C₆alkynyl; wherein each alkyl and alkynyl is independently optionally substituted with one or more R^{5a}; or two R⁵ on the same atom are taken together to form an oxo. In some embodiments, each R⁵ is independently —OH, —NR^{c}R^{d}, C₁-C₆alkyl, C₁-C₆haloalkyl, C₁-C₆hydroxyalkyl, or C₂-C₆alkynyl; wherein each alkyl and alkynyl is independently optionally substituted with one or more R^{5a}; or two R⁵ on the same atom are taken together to form an oxo. In some embodiments, each R⁵ is independently 3- to 6-membered cycloalkyl, or 3- to 6-membered heterocycloalkyl wherein each cycloalkyl and heterocycloalkyl is independently optionally substituted with one or more R^{5a}. In some embodiments, each R⁵ is independently —F, —C₁, —Br, —I, —OH, —NH₂, —CN, —CH₂OH, —CH₂CH₂OH, —CH₂CH₂OCH₃, —C(═O)CH₂OH, —CHF₂, —CH₃, —NH₂, —CH₂CN, cyclobutyl-OH, cyclopropyl-OH, azetidinyl-OH, or —CH₂C(═O)NH₂; or two R⁵ on the same atom are taken together to form an oxo. In some embodiments, each R⁵ is independently —F, —C₁, —Br, —I, —OH, —NH₂, —CN, —CH₂OH, —CH₂CH₂OH, —CH₂CH₂OCH₃, —C(═O)CH₂OH, —CHF₂, —CH₃, —NH₂, —CH₂CN, cyclopropyl-OH, azetidinyl-OH, or —CH₂C(═O)NH₂; or two R⁵ on the same atom are taken together to form an oxo. In some embodiments, each R⁵ is independently —OH, —CN, —CH₂OH, —CH₂CH₂OH, —CHF₂, —CH₃, —NH₂, —CH₂CN; or two R⁵ on the same atom are taken together to form an oxo. In some embodiments, —CH₃ is —CD₃.

In some embodiments of a compound of Formula (I), (Ia), (Ib), (Ic-1), (Ic-2), (Id-1), (Id-2), (Id-3), (Id-4), (Ic-1), or (Ie-2), each R^{5a} is independently halogen, —CN, —OH, —OR^{a}, —NR^{c}R^{d}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, each R^{5a} is independently halogen, —CN, —OH, —OR^{a}, —NR^{c}R^{d}, —C(═O)NR^{c}R^{d}, C₁-C₆alkyl, or C₁-C₆haloalkyl. In some embodiments, each R^{5a} is independently halogen, —CN, —OH, —OR^{a}, —NR^{c}R^{d}, or —C(═O)NR^{c}R^{d}. In some embodiments, each R^{5a} is independently —CN, —OH, or —C(═O)NH₂. In some embodiments of a compound of Formula (I), (Ia), or (Ib)-(Ie), each R^{5a} is independently —CN.

In some embodiments of a compound of Formula (I), (Ia), (Ib), (Ic-1), (Ic-2), (Id-1), (Id-2), (Id-3), (Id-4), (Ie-1), or (Ie-2), n is 0, 1, or 2. In some embodiments, n is 0 or 1. In some embodiments, n is 1 or 2. In some embodiments, n is 0. In some embodiments, n is 1. In some embodiments, n is 2. In some embodiments, n is 3. In some embodiments, n is 4.

In some embodiments of a compound of Formula (I), (Ia), (Ib), (Ic-1), (Ic-2), (Id-1), (Id-2), (Id-3), (Id-4), (Ie-1), or (Ie-2),

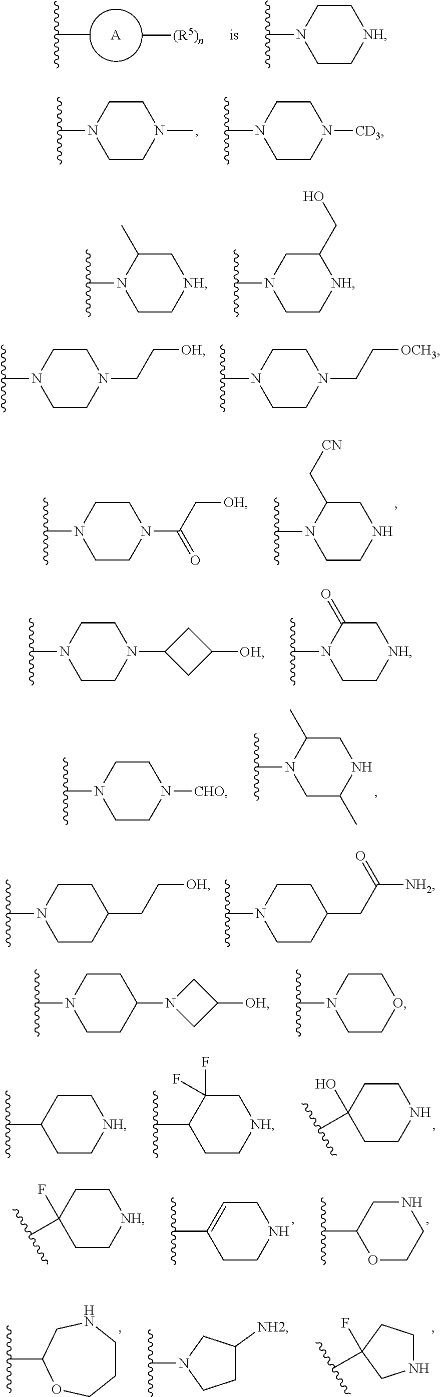

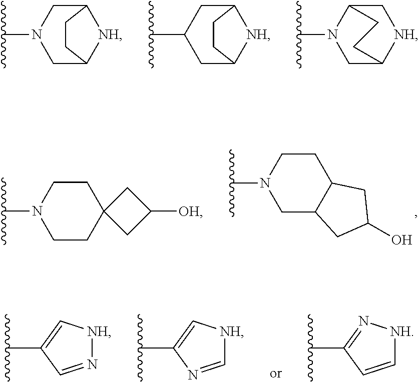

In some embodiments of a compound of Formula (I), (Ia), (Ib), (Ic-1), (Ic-2), (Id-1), (Id-2), (Id-3), (Id-4), (Ie-1), or (Ie-2),

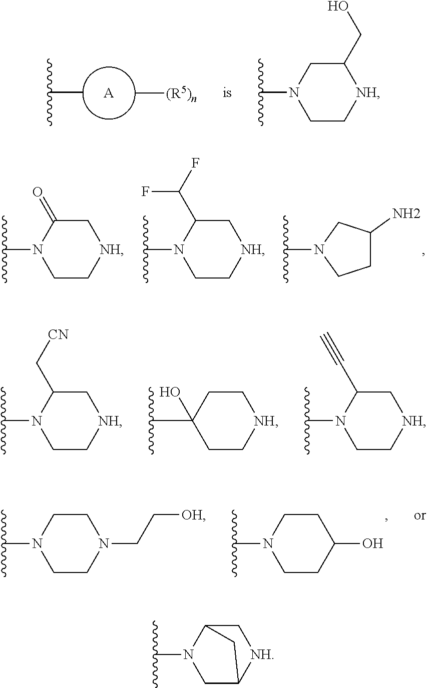

In some embodiments of a compound of Formula (I), (Ia), (Ib), (Ic-1), (Ic-2), (Id-1), (Id-2), (Id-3), (Id-4), (Ie-1), or (Ie-2), Ring B is cycloalkyl or heterocycloalkyl. In some embodiments, Ring B is 5- to 7-membered cycloalkyl. In some embodiments, Ring B is heterocycloalkyl. In some embodiments, Ring B is 5- to 7-membered heterocycloalkyl. In some embodiments, Ring B is a monocyclic or bicyclic 3- to 10-membered heterocycloalkyl comprising one, two, or three heteroatoms selected from the group consisting of N, O, S, and P. In some embodiments, Ring B is a monocyclic or bicyclic 3- to 10-membered heterocycloalkyl comprising one, two, or three heteroatoms selected from the group consisting of N, O, and S. In some embodiments, Ring B is a monocyclic or bicyclic 3- to 10-membered heterocycloalkyl comprising one, two, or three heteroatoms selected from the group consisting of N and O. In some embodiments, Ring B is a monocyclic 5- to 7-membered heterocycloalkyl comprising one or two heteroatoms selected from the group consisting of N, O, S, and P. In some embodiments, Ring B is a monocyclic 5- to 7-membered heterocycloalkyl comprising one or two heteroatoms selected from the group consisting of N, O, and S. In some embodiments, Ring B is a monocyclic 5- to 7-membered heterocycloalkyl comprising one or two heteroatoms selected from the group consisting of N and O. In some embodiments, Ring B is tetrahydropyranyl, tetrahydrothiopyranyl, or phosphinanyl. In some embodiments, Ring B is tetrahydropyranyl or tetrahydrothiopyranyl. In some embodiments, Ring B is tetrahydropyranyl. In some embodiments, Ring B is

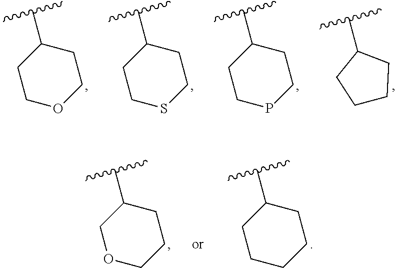

In some embodiments, Ring B is a bicyclic heterocycloalkyl. In some embodiments, Ring B is a fused bicyclic heterocycloalkyl. In some embodiments, Ring B is a spiro bicyclic heterocycloalkyl. In some embodiments, Ring B is a bridged bicyclic heterocycloalkyl. In some embodiments, Ring B is a bicyclic 6- to 10-membered heterocycloalkyl. In some embodiments, Ring B is a bicyclic 6- to 10-membered heterocycloalkyl comprising one or two heteroatoms selected from the group consisting of N, O, S, and P. In some embodiments, Ring B is a bicyclic 6- to 10-membered heterocycloalkyl comprising one or two heteroatoms selected from the group consisting of N, O, and S. In some embodiments, Ring B is a bicyclic 6- to 10-membered heterocycloalkyl comprising one or two heteroatoms selected from the group consisting of N and O. In some embodiments, Ring B is a bicyclic 8- to 9-membered heterocycloalkyl comprising one or two heteroatoms selected from the group consisting of N and O. In some embodiments, Ring B is

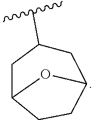 [SMILES: *C1CC2CCC(C1)O2]

In some embodiments, Ring B is

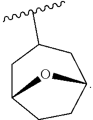 [SMILES: *C1C[C@H]2CC[C@@H](C1)O2]

In some embodiments of a compound of Formula (I), (Ia), (Ib), (Ic-1), (Ic-2), (Id-1), (Id-2), (Id-3), (Id-4), (Ie-1), or (Ie-2), each R⁶ is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl; wherein each alkyl, cycloalkyl, and heterocycloalkyl is independently optionally substituted with one or more R^{6a}; or two R⁶ on the same atom are taken together to form an oxo; or two R⁶ on the same atom are taken together to form ═NH. In some embodiments, each R⁶ is independently halogen, —CN, —OH, —OR^{a}, —NR^{c}R^{d}, C₁-C₆alkyl, C₁-C₆haloalkyl, cycloalkyl, or heterocycloalkyl; wherein each alkyl, cycloalkyl, and heterocycloalkyl is independently optionally substituted with one or more R^{6a}; or two R⁶ on the same atom are taken together to form an oxo; or two R⁶ on the same atom are taken together to form-NH. In some embodiments, each R⁶ is independently halogen, —CN, —OH, —OR^{a}, —NR^{c}R^{d}, C₁-C₆alkyl, or C₁-C₆haloalkyl; or two R⁶ on the same atom are taken together to form an oxo; or two R⁶ on the same atom are taken together to form ═NH. In some embodiments, each R⁶ is independently halogen, —CN, —OH, —OR^{a}, —NR^{c}R^{d}, C₁-C₆alkyl, or C₁-C₆haloalkyl. In some embodiments, each R⁶ is independently —OH or C₁-C₆alkyl; or two R⁶ on the same atom are taken together to form an oxo. In some embodiments, each R⁶ is independently —OH or C₁-C₆alkyl.

In some embodiments of a compound of Formula (I), (Ia), (Ib), (Ic-1), (Ic-2), (Id-1), (Id-2), (Id-3), (Id-4), (Ie-1), or (Ie-2), two R⁶ on the same atom are taken together to form an oxo.

In some embodiments of a compound of Formula (I), (Ia), (Ib), (Ic-1), (Ic-2), (Id-1), (Id-2), (Id-3), (Id-4), (Ie-1), or (Ie-2), two R⁶ on the same atom are taken together to form ═NH.

In some embodiments of a compound of Formula (I), (Ia), (Ib), (Ic-1), (Ic-2), (Id-1), (Id-2), (Id-3), (Id-4), (Ie-1), or (Ie-2), each R^{6a} is independently halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, each R^{6a} is independently halogen, —CN, —OH, —OR^{a}, —NR^{c}R^{d}, C₁-C₆alkyl, C₁-C₆haloalkyl, C₁-C₆hydroxyalkyl, C₁-C₆aminoalkyl, C₁-C₆heteroalkyl, cycloalkyl, or heterocycloalkyl. In some embodiments, each R^{6a} is independently halogen, —CN, —OH, —OR^{a}, —NR^{c}R^{d}, C₁-C₆alkyl, or C₁-C₆haloalkyl.

In some embodiments of a compound of Formula (I), (Ia), (Ib), (Ic-1), (Ic-2), (Id-1), (Id-2), (Id-3), (Id-4), (Ie-1), or (Ie-2), m is 0, 1, 2, 3, or 4. In some embodiments, m is 0, 1, 2, or 3. In some embodiments, m is 1, 2, 3, or 4. In some embodiments, m is 0, 1, or 2. In some embodiments, m is 2, 3, or 4. In some embodiments, m is 0. In some embodiments, m is 1. In some embodiments, m is 2. In some embodiments, m is 3. In some embodiments, m is 4. In some embodiments, m is 3 or 4.

In some embodiments of a compound of Formula (I), (Ia), (Ib), (Ic-1), (Ic-2), (Id-1), (Id-2), (Id-3), (Id-4), (Ie-1), or (Ie-2).

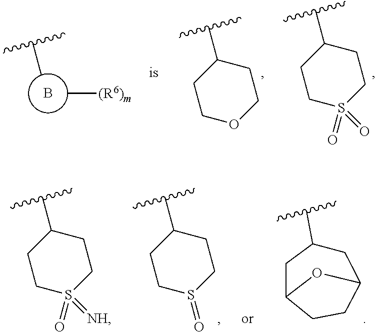

In some embodiments,

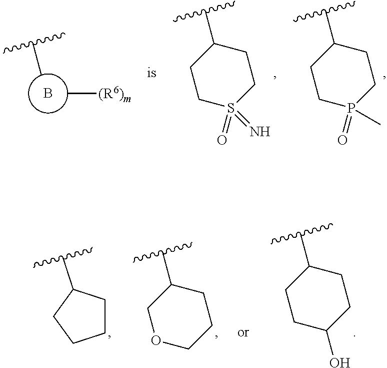

In some embodiments,

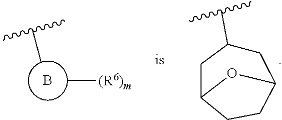

In some embodiments,

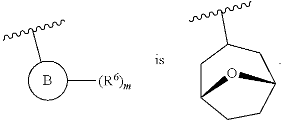

In some embodiments of a compound disclosed herein, each R^{a} is independently C₁-C₆alkyl, C₁-C₆haloalkyl, C₁-C₆hydroxyalkyl, C₁-C₆aminoalkyl, C₁-C₆heteroalkyl, -L-cycloalkyl, -L-heterocycloalkyl,-L-aryl, or -L-heteroaryl, wherein each alkyl, cycloalkyl, heterocycloalkyl, aryl, and heteroaryl is independently optionally substituted with one or more R. In some embodiments of a compound disclosed herein, each R^{a} is independently -L-cycloalkyl, -L-heterocycloalkyl, -L-aryl, or -L-heteroaryl, wherein each cycloalkyl, heterocycloalkyl, aryl, and heteroaryl is independently optionally substituted with one or more R. In some embodiments of a compound disclosed herein, each R^{a} is independently -L-cycloalkyl or -L-heterocycloalkyl, wherein each cycloalkyl and heterocycloalkyl is independently optionally substituted with one or more R. In some embodiments of a compound disclosed herein, each R^{a} is independently C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, each R^{a} is independently C₁-C₆alkyl or C₁-C₆haloalkyl, wherein each alkyl is independently optionally substituted with one or more R. In some embodiments of a compound disclosed herein, each R^{a} is independently C₁-C₆alkyl or C₁-C₆haloalkyl. In some embodiments of a compound disclosed herein, each R^{a} is independently C₁-C₆alkyl. In some embodiments of a compound disclosed herein, each R^{a} is independently C₁-C₆haloalkyl.

In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, C₁-C₆heteroalkyl, -L-cycloalkyl, -L-heterocycloalkyl, -L-aryl, or -L-heteroaryl, wherein each alkyl, cycloalkyl, heterocycloalkyl, aryl, and heteroaryl is independently optionally substituted with one or more R. In some embodiments of a compound disclosed herein, each R^{b} is independently hydrogen, -L-cycloalkyl, -L-heterocycloalkyl, -L-aryl, or -L-heteroaryl, wherein each cycloalkyl, heterocycloalkyl, aryl, and heteroaryl is independently optionally substituted with one or more R. In some embodiments of a compound disclosed herein, each R^{b} is independently hydrogen, -L-cycloalkyl or -L-heterocycloalkyl, wherein each cycloalkyl and heterocycloalkyl is independently optionally substituted with one or more R. In some embodiments of a compound disclosed herein, each R^{b} is independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R. In some embodiments of a compound disclosed herein, each R^{b} is independently hydrogen, C₁-C₆alkyl, or C₁-C₆haloalkyl, wherein each alkyl is independently optionally substituted with one or more R. In some embodiments of a compound disclosed herein, each R^{b} is independently hydrogen, C₁-C₆alkyl, or C₁-C₆haloalkyl. In some embodiments of a compound disclosed herein, each R^{b} is independently hydrogen or C₁-C₆alkyl. In some embodiments of a compound disclosed herein, each R^{b} is independently hydrogen or C₁-C₆haloalkyl. In some embodiments of a compound disclosed herein, each R^{b} is hydrogen. In some embodiments of a compound disclosed herein, each R^{b} is independently C₁-C₆alkyl. In some embodiments of a compound disclosed herein, each R^{b} is independently C₁-C₆haloalkyl.

In some embodiments of a compound disclosed herein, Re and R^{d} are each independently hydrogen, C₁-C₆alkyl, C₁-C₆haloalkyl, C₁-C₆hydroxyalkyl, C₁-C₆aminoalkyl, C₁-C₆heteroalkyl, -L-cycloalkyl, -L-heterocycloalkyl, -L-aryl, or -L-heteroaryl, wherein each alkyl, cycloalkyl, heterocycloalkyl, aryl, and heteroaryl is independently optionally substituted with one or more R. In some embodiments of a compound disclosed herein, Re and R^{d} are each independently hydrogen, -L-cycloalkyl, -L-heterocycloalkyl, -L-aryl, or -L-heteroaryl, wherein each cycloalkyl, heterocycloalkyl, aryl, and heteroaryl is independently optionally substituted with one or more R. In some embodiments of a compound disclosed herein, Re and R^{d} are each independently hydrogen, -L-cycloalkyl or -L-heterocycloalkyl, wherein each cycloalkyl and heterocycloalkyl is independently optionally substituted with one or more R. In some embodiments of a compound disclosed herein, Re and R^{d} are each independently hydrogen, C₁-C₆alkyl, C₁-C₆haloalkyl, C₁-C₆hydroxyalkyl, C₁-C₆aminoalkyl, or C₁-C₆heteroalkyl, wherein each alkyl is independently optionally substituted with one or more R.

In some embodiments of a compound disclosed herein, Re and R^{d} are each independently hydrogen, C₁-C₆alkyl, or C₁-C₆haloalkyl, wherein each alkyl is independently optionally substituted with one or more R. In some embodiments of a compound disclosed herein, Re and R^{d} are each independently hydrogen, C₁-C₆alkyl, or C₁-C₆haloalkyl. In some embodiments of a compound disclosed herein, Re and R^{d} are each independently hydrogen or C₁-C₆alkyl. In some embodiments of a compound disclosed herein, Re and R^{d} are each independently hydrogen or C₁-C₆haloalkyl. In some embodiments of a compound disclosed herein, Re and R^{d} are each hydrogen. In some embodiments of a compound disclosed herein, Re and R^{d} are each independently C₁-C₆alkyl. In some embodiments of a compound disclosed herein, Re and R^{d} are each independently C₁-C₆haloalkyl.

In some embodiments of a compound disclosed herein, Re and R^{d} are taken together with the atom to which they are attached to form a heterocycloalkyl optionally substituted with one or more R; and

In some embodiments of a compound disclosed herein, L is absent. In some embodiments of a compound disclosed herein, L is C₁-C₃ alkylene optionally substituted with one or more R. In some embodiments of a compound disclosed herein, Lis C₁-C₃ alkylene. In some embodiments of a compound disclosed herein, L is —CH₂—. In some embodiments of a compound disclosed herein, L is —CH₂CH₂—. In some embodiments of a compound disclosed herein, L is —CH₂CH₂CH₂—.

In some embodiments of a compound disclosed herein, each R is independently halogen, —CN, —OH, —SF⁵, —NH₂, —NHC₁-C₃alkyl, —N(C₁-C₃alkyl)₂, —C(═O) C₁-C₃alkyl, —C(═O)OH, —C(═O)OC₁-C₃alkyl, —C(═O)NH₂, —C(═O)NHC₁-C₃alkyl, —C(═O) N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl. In some embodiments of a compound disclosed herein, each R is independently halogen, —CN, —OH, —SF⁵, —NH₂, —NHC₁-C₃alkyl, —N(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₃cycloalkyl. In some embodiments of a compound disclosed herein, each R is independently halogen, —CN, —OH, —SF⁵, —NH₂, —NHC₁-C₃alkyl, —N(C₁-C₃alkyl)₂, —C(═O) C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, C₁-C₃alkyl, or C₁-C₃haloalkyl. In some embodiments of a compound disclosed herein, each R is independently halogen, —CN, —OH, —SF⁵, C₁-C₃alkyl, C₁-C₃alkoxy, C₁-C₃haloalkyl, C₁-C₃haloalkoxy, C₁-C₃hydroxyalkyl, C₁-C₆aminoalkyl, C₁-C₃heteroalkyl, or C₃-C₃cycloalkyl. In some embodiments of a compound disclosed herein, each R is independently halogen, —CN, —OH, —SF⁵, C₁-C₃alkyl, C₁-C₃alkoxy, or C₁-C₃haloalkyl. In some embodiments of a compound disclosed herein, each R is independently halogen, C₁-C₃alkyl, or C₁-C₃haloalkyl.

In some embodiments of a compound disclosed herein, one or more of R¹, R², R³, R₄, R^{Z1}, R^{Z2}, R⁵, R^{5a}, R⁶, R^{6a}, R^{a}, R^{b}, R^{c}, R^{d}, L, and R groups comprise deuterium at a percentage higher than the natural abundance of deuterium.

In some embodiments of a compound disclosed herein, one or more ¹H are replaced with one or more deuteriums in one or more of the following groups R¹, R², R³, R⁴, R^{Z1}, R^{Z2}, R⁵, R^{5a}, R⁶, R^{6a}, R^{a}, R^{b}, R^{c}, R^{d}, L, and R.

In some embodiments of a compound disclosed herein, the abundance of deuterium in each of R¹, R², R³, R⁴, R^{Z1}, R^{Z2}, R⁵, R^{5a}, R⁶, R^{6a}, R^{a}, R^{b}, R^{c}, R^{d}, L, and R is independently at least 1%, at least 10%, at least 20%, at least 30%, at least 40%, at least 50%, at least 60%, at least 70%, at least 80%, at least 90%, or 100% by molar.

In some embodiments of a compound disclosed herein, one or more 1H of Ring A or Ring B are replaced with one or more deuteriums.

Any combination of the groups described above for the various variables is contemplated herein. Throughout the specification, groups and substituents thereof are chosen by one skilled in the field to provide stable moieties and compounds.

In some embodiments the compound disclosed herein, or a pharmaceutically acceptable salt, or stereoisomer thereof, is one of the compounds in Table 1.

TABLE 1

| Ex. | Structure |
|---|---|
| 1 | 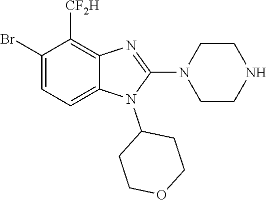 [SMILES: FC(F)c1c(Br)ccc2c1nc(N1CCNCC1)n2C1CCOCC1] |
| 3 | 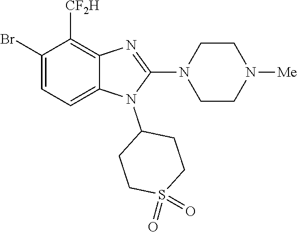 [SMILES: CN1CCN(c2nc3c(C(F)F)c(Br)ccc3n2C2CCS(=O)(=O)CC2)CC1] |
| 2 | 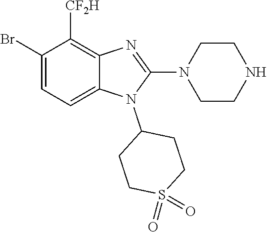 [SMILES: O=S1(=O)CCC(n2c(N3CCNCC3)nc3c(C(F)F)c(Br)ccc32)CC1] |
| 4 | 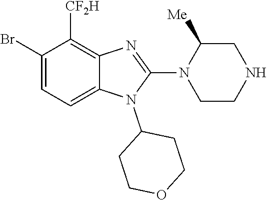 [SMILES: C[C@H]1CNCCN1c1nc2c(C(F)F)c(Br)ccc2n1C1CCOCC1] |
| 5 | 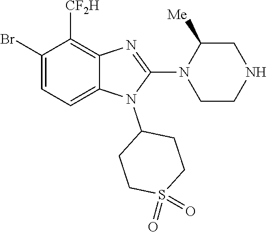 [SMILES: C[C@H]1CNCCN1c1nc2c(C(F)F)c(Br)ccc2n1C1CCS(=O)(=O)CC1] |
| 6 | 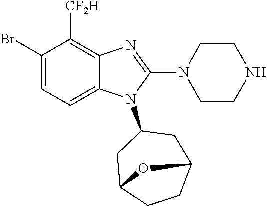 [SMILES: FC(F)c1c(Br)ccc2c1nc(N1CCNCC1)n2[C@@H]1C[C@H]2CC[C@@H](C1)O2] |
| 7 | 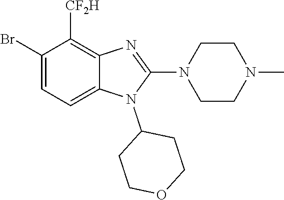 [SMILES: CN1CCN(c2nc3c(C(F)F)c(Br)ccc3n2C2CCOCC2)CC1] |
| 8 | 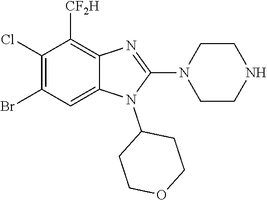 [SMILES: FC(F)c1c(Cl)c(Br)cc2c1nc(N1CCNCC1)n2C1CCOCC1] |
| 9 | 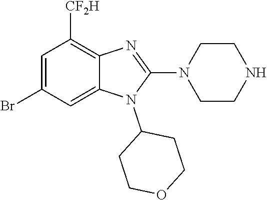 [SMILES: FC(F)c1cc(Br)cc2c1nc(N1CCNCC1)n2C1CCOCC1] |
| 10 | 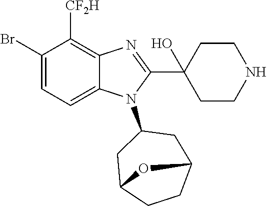 [SMILES: OC1(c2nc3c(C(F)F)c(Br)ccc3n2[C@@H]2C[C@H]3CC[C@@H](C2)O3)CCNCC1] |
| 11 | 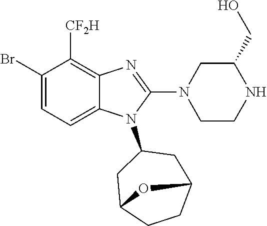 [SMILES: OC[C@H]1CN(c2nc3c(C(F)F)c(Br)ccc3n2[C@@H]2C[C@H]3CC[C@@H](C2)O3)CCN1] |
| 12 | 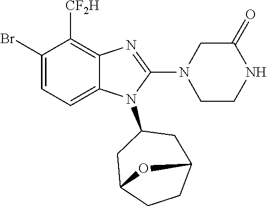 [SMILES: O=C1CN(c2nc3c(C(F)F)c(Br)ccc3n2[C@@H]2C[C@H]3CC[C@@H](C2)O3)CCN1] |
| 13 | 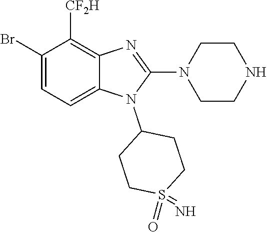 [SMILES: N=S1(=O)CCC(n2c(N3CCNCC3)nc3c(C(F)F)c(Br)ccc32)CC1] |
| 14 | 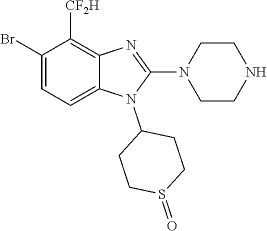 [SMILES: O=S1CCC(n2c(N3CCNCC3)nc3c(C(F)F)c(Br)ccc32)CC1] |
| 15 | 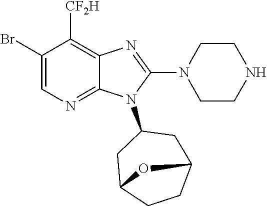 [SMILES: FC(F)c1c(Br)cnc2c1nc(N1CCNCC1)n2[C@@H]1C[C@H]2CC[C@@H](C1)O2] |
| 16 | 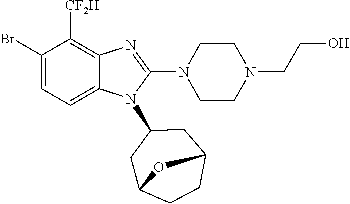 [SMILES: OCCN1CCN(c2nc3c(C(F)F)c(Br)ccc3n2[C@@H]2C[C@H]3CC[C@@H](C2)O3)CC1] |
| 17 | 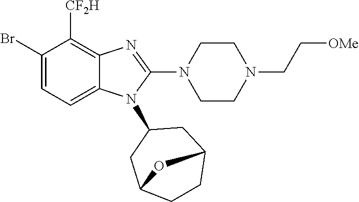 [SMILES: COCCN1CCN(c2nc3c(C(F)F)c(Br)ccc3n2[C@@H]2C[C@H]3CC[C@@H](C2)O3)CC1] |
| 18 | 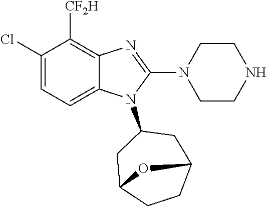 [SMILES: FC(F)c1c(Cl)ccc2c1nc(N1CCNCC1)n2[C@@H]1C[C@H]2CC[C@@H](C1)O2] |
| 19 | 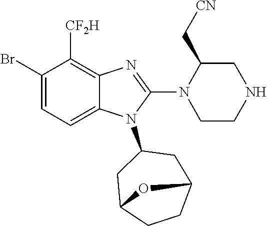 [SMILES: N#CC[C@H]1CNCCN1c1nc2c(C(F)F)c(Br)ccc2n1[C@@H]1C[C@H]2CC[C@@H](C1)O2] |
| 20 | 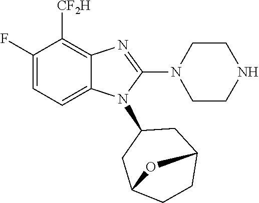 [SMILES: Fc1ccc2c(nc(N3CCNCC3)n2[C@@H]2C[C@H]3CC[C@@H](C2)O3)c1C(F)F] |
| 21 | 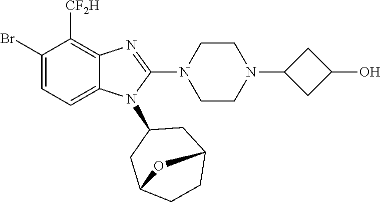 [SMILES: OC1CC(N2CCN(c3nc4c(C(F)F)c(Br)ccc4n3[C@@H]3C[C@H]4CC[C@@H](C3)O4)CC2)C1] |
| 22 | 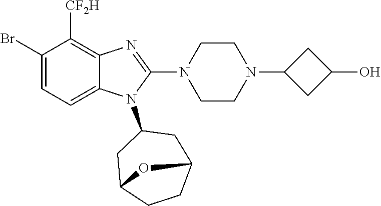 [SMILES: OC1CC(N2CCN(c3nc4c(C(F)F)c(Br)ccc4n3[C@@H]3C[C@H]4CC[C@@H](C3)O4)CC2)C1] |
| 23 | 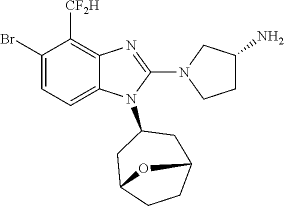 [SMILES: N[C@@H]1CCN(c2nc3c(C(F)F)c(Br)ccc3n2[C@@H]2C[C@H]3CC[C@@H](C2)O3)C1] |
| 24 | 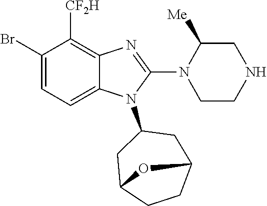 [SMILES: C[C@H]1CNCCN1c1nc2c(C(F)F)c(Br)ccc2n1[C@@H]1C[C@H]2CC[C@@H](C1)O2] |
| 25 | 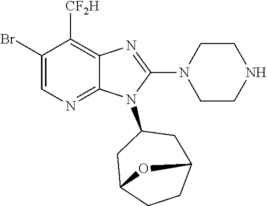 [SMILES: FC(F)c1c(Br)cnc2c1nc(N1CCNCC1)n2[C@@H]1C[C@H]2CC[C@@H](C1)O2] |
| 26 | 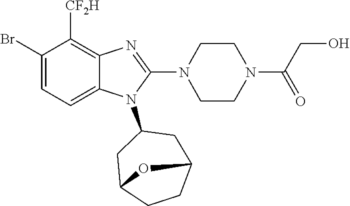 [SMILES: O=C(CO)N1CCN(c2nc3c(C(F)F)c(Br)ccc3n2[C@@H]2C[C@H]3CC[C@@H](C2)O3)CC1] |
| 27 | 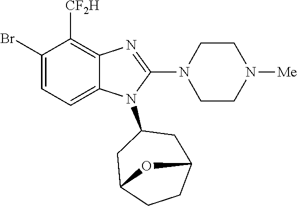 [SMILES: CN1CCN(c2nc3c(C(F)F)c(Br)ccc3n2[C@@H]2C[C@H]3CC[C@@H](C2)O3)CC1] |
| 28 | 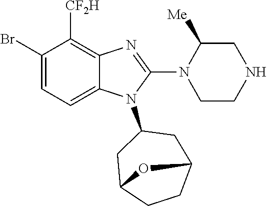 [SMILES: C[C@H]1CNCCN1c1nc2c(C(F)F)c(Br)ccc2n1[C@@H]1C[C@H]2CC[C@@H](C1)O2] |
| 29 | 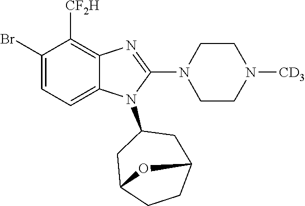 [SMILES: [2H]C([2H])([2H])N1CCN(c2nc3c(C(F)F)c(Br)ccc3n2[C@@H]2C[C@H]3CC[C@@H](C2)O3)CC1] |
| 30 | 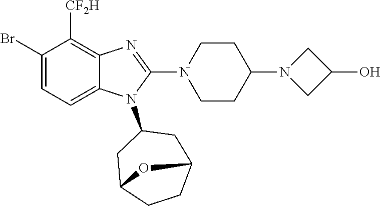 [SMILES: OC1CN(C2CCN(c3nc4c(C(F)F)c(Br)ccc4n3[C@@H]3C[C@H]4CC[C@@H](C3)O4)CC2)C1] |
| 31 | 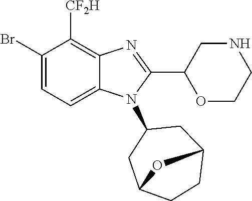 [SMILES: FC(F)c1c(Br)ccc2c1nc(C1CNCCO1)n2[C@@H]1C[C@H]2CC[C@@H](C1)O2] |
| 32 | 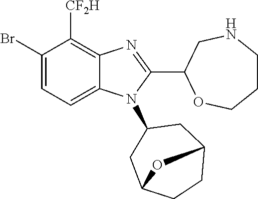 [SMILES: FC(F)c1c(Br)ccc2c1nc(C1CNCCCO1)n2[C@@H]1C[C@H]2CC[C@@H](C1)O2] |
| 33 | 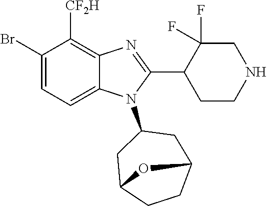 [SMILES: FC(F)c1c(Br)ccc2c1nc(C1CCNCC1(F)F)n2[C@@H]1C[C@H]2CC[C@@H](C1)O2] |
| 34 | 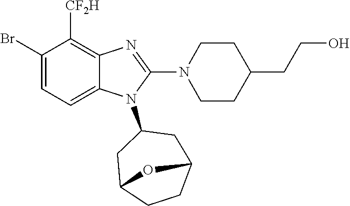 [SMILES: OCCC1CCN(c2nc3c(C(F)F)c(Br)ccc3n2[C@@H]2C[C@H]3CC[C@@H](C2)O3)CC1] |
| 35 | 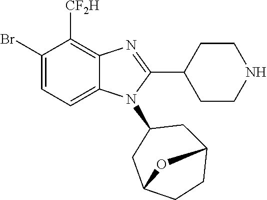 [SMILES: FC(F)c1c(Br)ccc2c1nc(C1CCNCC1)n2[C@@H]1C[C@H]2CC[C@@H](C1)O2] |
| 36 | 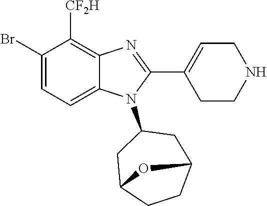 [SMILES: FC(F)c1c(Br)ccc2c1nc(C1=CCNCC1)n2[C@@H]1C[C@H]2CC[C@@H](C1)O2] |
| 37 | 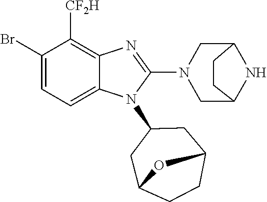 [SMILES: FC(F)c1c(Br)ccc2c1nc(N1CC3CCC(C1)N3)n2[C@@H]1C[C@H]2CC[C@@H](C1)O2] |
| 38 | 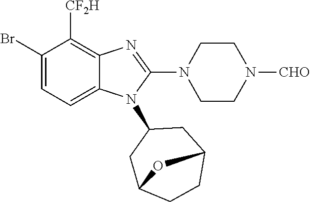 [SMILES: O=CN1CCN(c2nc3c(C(F)F)c(Br)ccc3n2[C@@H]2C[C@H]3CC[C@@H](C2)O3)CC1] |
| 39 | 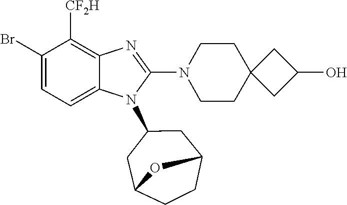 [SMILES: OC1CC2(CCN(c3nc4c(C(F)F)c(Br)ccc4n3[C@@H]3C[C@H]4CC[C@@H](C3)O4)CC2)C1] |
| 40 | 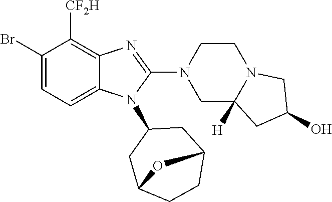 [SMILES: O[C@H]1C[C@@H]2CN(c3nc4c(C(F)F)c(Br)ccc4n3[C@@H]3C[C@H]4CC[C@@H](C3)O4)CCN2C1] |
| 41 | 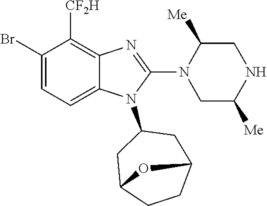 [SMILES: C[C@H]1CN(c2nc3c(C(F)F)c(Br)ccc3n2[C@@H]2C[C@H]3CC[C@@H](C2)O3)[C@@H](C)CN1] |
| 42 | 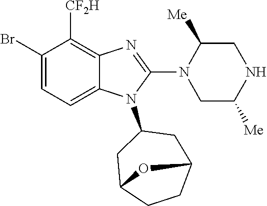 [SMILES: C[C@@H]1CN(c2nc3c(C(F)F)c(Br)ccc3n2[C@@H]2C[C@H]3CC[C@@H](C2)O3)[C@@H](C)CN1] |
| 43 | 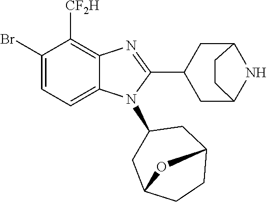 [SMILES: FC(F)c1c(Br)ccc2c1nc(C1CC3CCC(C1)N3)n2[C@@H]1C[C@H]2CC[C@@H](C1)O2] |
| 44 | 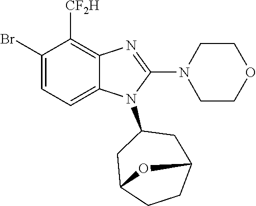 [SMILES: FC(F)c1c(Br)ccc2c1nc(N1CCOCC1)n2[C@@H]1C[C@H]2CC[C@@H](C1)O2] |
| 45 | 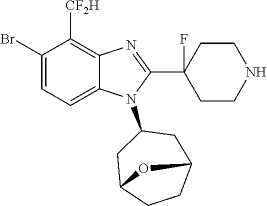 [SMILES: FC(F)c1c(Br)ccc2c1nc(C1(F)CCNCC1)n2[C@@H]1C[C@H]2CC[C@@H](C1)O2] |
| 46 | 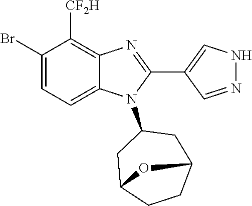 [SMILES: FC(F)c1c(Br)ccc2c1nc(-c1cn[nH]c1)n2[C@@H]1C[C@H]2CC[C@@H](C1)O2] |
| 47 | 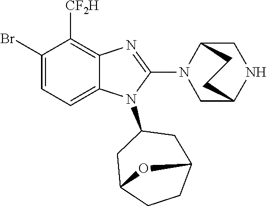 [SMILES: FC(F)c1c(Br)ccc2c1nc(N1C[C@@H]3CC[C@H]1CN3)n2[C@@H]1C[C@H]2CC[C@@H](C1)O2] |
| 48 | 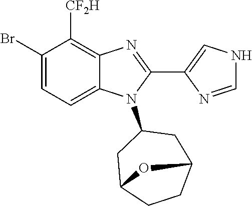 [SMILES: FC(F)c1c(Br)ccc2c1nc(-c1c[nH]cn1)n2[C@@H]1C[C@H]2CC[C@@H](C1)O2] |
| 49 | 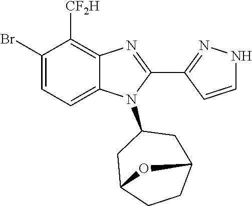 [SMILES: FC(F)c1c(Br)ccc2c1nc(-c1cc[nH]n1)n2[C@@H]1C[C@H]2CC[C@@H](C1)O2] |
| 50 | 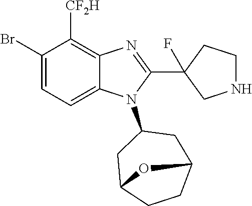 [SMILES: FC(F)c1c(Br)ccc2c1nc(C1(F)CCNC1)n2[C@@H]1C[C@H]2CC[C@@H](C1)O2] |
| 51 | 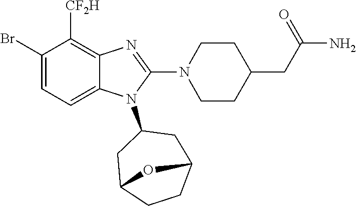 [SMILES: NC(=O)CC1CCN(c2nc3c(C(F)F)c(Br)ccc3n2[C@@H]2C[C@H]3CC[C@@H](C2)O3)CC1] |

In some embodiments the compound disclosed herein, or a pharmaceutically acceptable salt, or stereoisomer thereof, is one of the compounds in Table 2.

TABLE 2

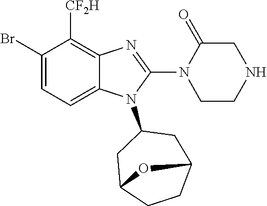 [SMILES: O=C1CNCCN1c1nc2c(C(F)F)c(Br)ccc2n1[C@@H]1C[C@H]2CC[C@@H](C1)O2]

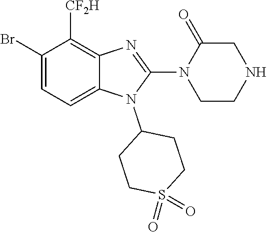 [SMILES: O=C1CNCCN1c1nc2c(C(F)F)c(Br)ccc2n1C1CCS(=O)(=O)CC1]

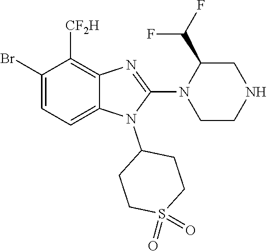 [SMILES: O=S1(=O)CCC(n2c(N3CCNC[C@@H]3C(F)F)nc3c(C(F)F)c(Br)ccc32)CC1]

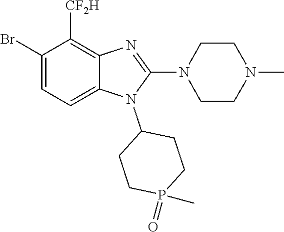 [SMILES: CN1CCN(c2nc3c(C(F)F)c(Br)ccc3n2C2CCP(C)(=O)CC2)CC1]

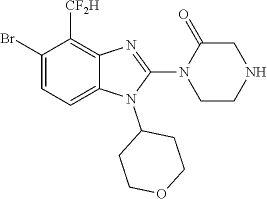 [SMILES: O=C1CNCCN1c1nc2c(C(F)F)c(Br)ccc2n1C1CCOCC1]

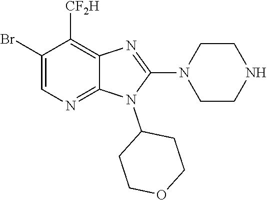 [SMILES: FC(F)c1c(Br)cnc2c1nc(N1CCNCC1)n2C1CCOCC1]

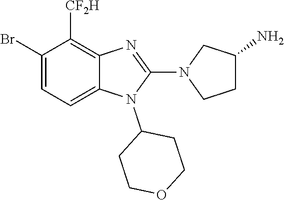 [SMILES: N[C@@H]1CCN(c2nc3c(C(F)F)c(Br)ccc3n2C2CCOCC2)C1]

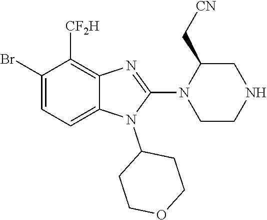 [SMILES: N#CC[C@H]1CNCCN1c1nc2c(C(F)F)c(Br)ccc2n1C1CCOCC1]

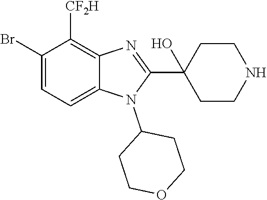 [SMILES: OC1(c2nc3c(C(F)F)c(Br)ccc3n2C2CCOCC2)CCNCC1]

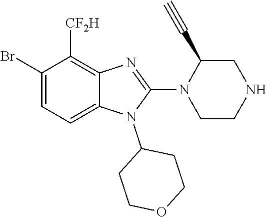 [SMILES: C#C[C@H]1CNCCN1c1nc2c(C(F)F)c(Br)ccc2n1C1CCOCC1]

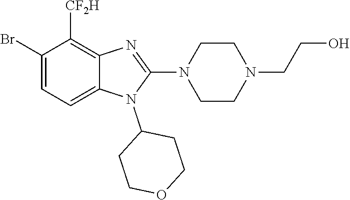 [SMILES: OCCN1CCN(c2nc3c(C(F)F)c(Br)ccc3n2C2CCOCC2)CC1]

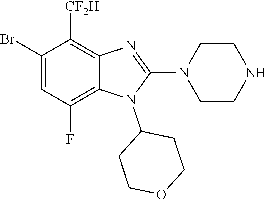 [SMILES: Fc1cc(Br)c(C(F)F)c2nc(N3CCNCC3)n(C3CCOCC3)c12]

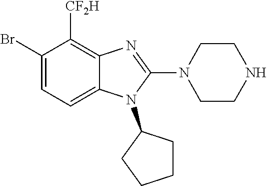

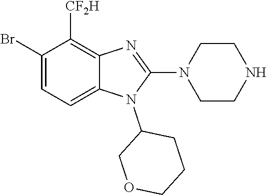 [SMILES: FC(F)c1c(Br)ccc2c1nc(N1CCNCC1)n2C1CCCOC1]

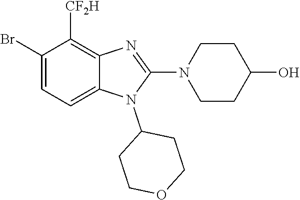 [SMILES: OC1CCN(c2nc3c(C(F)F)c(Br)ccc3n2C2CCOCC2)CC1]

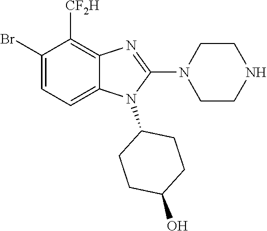 [SMILES: O[C@H]1CC[C@H](n2c(N3CCNCC3)nc3c(C(F)F)c(Br)ccc32)CC1]

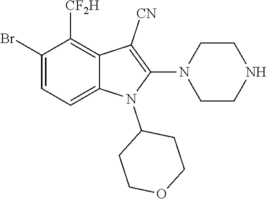 [SMILES: N#Cc1c(N2CCNCC2)n(C2CCOCC2)c2ccc(Br)c(C(F)F)c12]

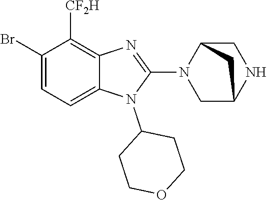 [SMILES: FC(F)c1c(Br)ccc2c1nc(N1C[C@@H]3C[C@H]1CN3)n2C1CCOCC1]

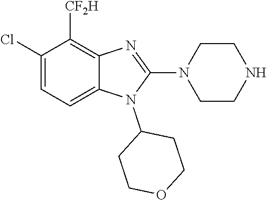 [SMILES: FC(F)c1c(Cl)ccc2c1nc(N1CCNCC1)n2C1CCOCC1]

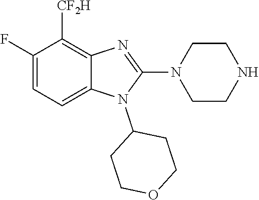 [SMILES: Fc1ccc2c(nc(N3CCNCC3)n2C2CCOCC2)c1C(F)F]

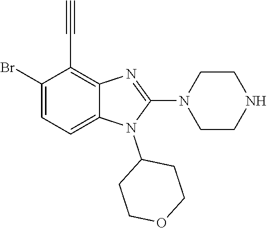 [SMILES: C#Cc1c(Br)ccc2c1nc(N1CCNCC1)n2C1CCOCC1]

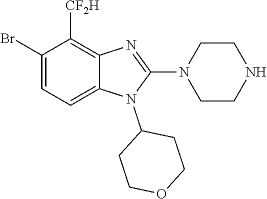 [SMILES: FC(F)c1c(Br)ccc2c1nc(N1CCNCC1)n2C1CCOCC1]

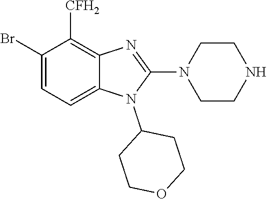

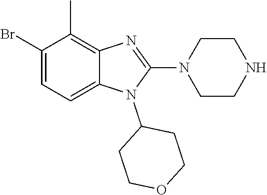 [SMILES: Cc1c(Br)ccc2c1nc(N1CCNCC1)n2C1CCOCC1]

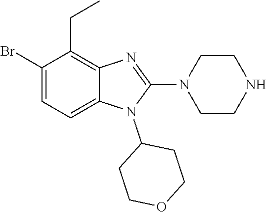 [SMILES: CCc1c(Br)ccc2c1nc(N1CCNCC1)n2C1CCOCC1]

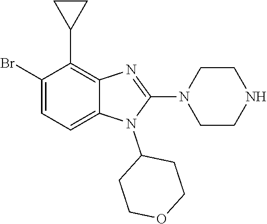 [SMILES: Brc1ccc2c(nc(N3CCNCC3)n2C2CCOCC2)c1C1CC1]

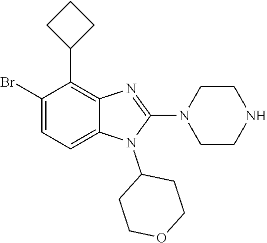 [SMILES: Brc1ccc2c(nc(N3CCNCC3)n2C2CCOCC2)c1C1CCC1]

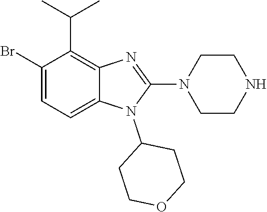 [SMILES: CC(C)c1c(Br)ccc2c1nc(N1CCNCC1)n2C1CCOCC1]

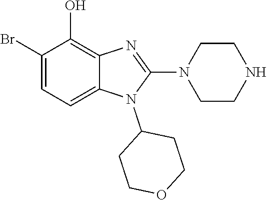 [SMILES: Oc1c(Br)ccc2c1nc(N1CCNCC1)n2C1CCOCC1]

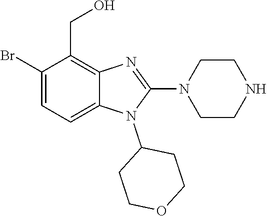 [SMILES: OCc1c(Br)ccc2c1nc(N1CCNCC1)n2C1CCOCC1]

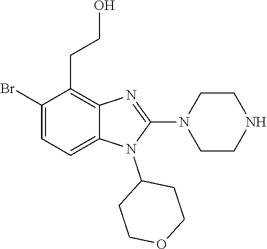 [SMILES: OCCc1c(Br)ccc2c1nc(N1CCNCC1)n2C1CCOCC1]

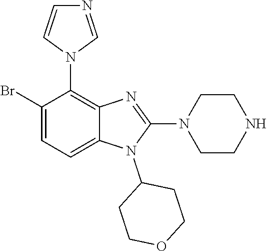 [SMILES: Brc1ccc2c(nc(N3CCNCC3)n2C2CCOCC2)c1-n1ccnc1]

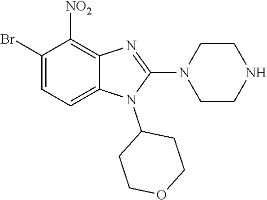 [SMILES: O=[N+]([O-])c1c(Br)ccc2c1nc(N1CCNCC1)n2C1CCOCC1]

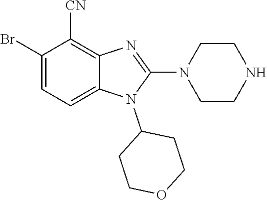 [SMILES: N#Cc1c(Br)ccc2c1nc(N1CCNCC1)n2C1CCOCC1]

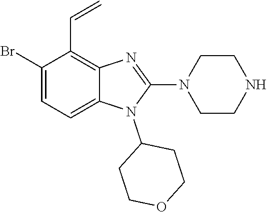 [SMILES: C=Cc1c(Br)ccc2c1nc(N1CCNCC1)n2C1CCOCC1]

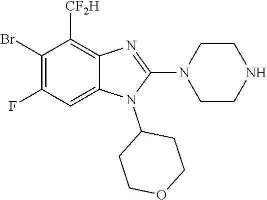 [SMILES: Fc1cc2c(nc(N3CCNCC3)n2C2CCOCC2)c(C(F)F)c1Br]

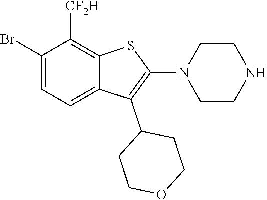 [SMILES: FC(F)c1c(Br)ccc2c(C3CCOCC3)c(N3CCNCC3)sc12]

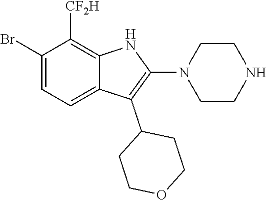 [SMILES: FC(F)c1c(Br)ccc2c(C3CCOCC3)c(N3CCNCC3)[nH]c12]

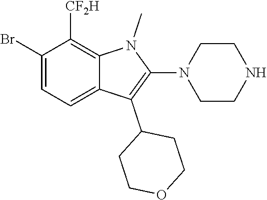 [SMILES: Cn1c(N2CCNCC2)c(C2CCOCC2)c2ccc(Br)c(C(F)F)c21]

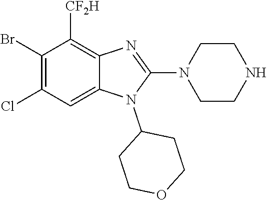 [SMILES: FC(F)c1c(Br)c(Cl)cc2c1nc(N1CCNCC1)n2C1CCOCC1]

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Isotopically Enriched Compounds

Unless otherwise stated, compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford some therapeutic advantages, such as increased in vivo half-life and/or exposure, or may provide a compound useful for investigating in vivo routes of drug elimination and metabolism.

For example, the compounds described herein may be artificially enriched in one or more particular isotopes. In some embodiments, the compounds described herein may be artificially enriched in one or more isotopes that are not predominantly found in nature. In some embodiments, the compounds described herein may be artificially enriched in one or more isotopes selected from deuterium (²H), tritium (³H), iodine-125 (¹²⁵I) or carbon-14 (¹⁴C). In some embodiments, the compounds described herein are artificially enriched in one or more isotopes selected from ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹³¹I, and ¹²⁵I. In some embodiments, the abundance of the enriched isotopes is independently at least 1%, at least 10%, at least 20%, at least 30%, at least 40%, at least 50%, at least 60%, at least 70%, at least 80%, at least 90%, or 100% by molar.

In some embodiments, the compound is deuterated in at least one position. In some embodiments, the compounds disclosed herein have some or all of the 1H atoms replaced with 2H atoms.

The methods of synthesis for deuterium-containing compounds are known in the art and include, by way of non-limiting example only, the procedure described in U.S. Pat. Nos. 5,846,514 and 6,334,997, and the following synthetic methods. For example, deuterium substituted compounds may be synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6 (10)] 2000, 110 pp; George W.; Varma, Rajender S. The Synthesis of Radiolabeled Compounds via Organometallic Intermediates, Tetrahedron, 1989, 45 (21), 6601-21; and Evans, E. Anthony. Synthesis of radiolabeled compounds, J. Radioanal. Chem., 1981, 64 (1-2), 9-32.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

Disclosed herein is method of modulating cyclic dependent kinase 8 and/or 19 (CDK8/19) activity in a subject, comprising administering to the subject a compound described herein, or a pharmaceutically acceptable salt, or stereoisomer thereof.

In another aspect, provided herein is a method of inhibiting cyclic dependent kinase 8 and/or 19 (CDK8/19) activity in a subject, comprising administering to the subject a compound described herein, or a pharmaceutically acceptable salt, or stereoisomer thereof.

In another aspect, provided herein is a method of treating cancer in a subject in need thereof, the method comprising administering a therapeutically effective amount of a compound described herein, or a pharmaceutically acceptable salt, or stereoisomer thereof, or a pharmaceutical composition described herein.

In some embodiments, the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM). In some embodiments, the cancer is leukemia. In some embodiments, the cancer is acute myeloid leukemia (AML). In some embodiments, the cancer is chronic myeloid leukemia. In some embodiments, the cancer is acute lymphoblastic leukemia (ALL). In some embodiments, the cancer is non-Hodgkin lymphoma (NHL). In some embodiments, the cancer is Hodgkin lymphoma (HL). In some embodiments, the cancer is multiple myeloma (MM).

In some embodiments, the cancer is a solid cancer.

In some embodiments, the cancer is a skin cancer, ocular cancer, gastrointestinal cancer, thyroid cancer, breast cancer, ovarian cancer, central nervous system cancer, laryngeal cancer, cervical cancer, lymphatic system cancer, genitourinary tract cancer, bone cancer, biliary tract cancer, pancreatic cancer, endometrial cancer, liver cancer, lung cancer, prostate cancer, or colon cancer. In some embodiments, the cancer is a prostate cancer, breast cancer, colon cancer, pancreatic cancer, skin cancer, ocular cancer, gastrointestinal cancer, thyroid cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, head and neck squamous cell carcinomas, or melanoma. In some embodiments, the cancer is a prostate cancer, breast cancer, colon cancer, pancreatic cancer, head and neck squamous cell carcinomas, or melanoma. In some embodiments, the cancer is a prostate cancer. In some embodiments, the cancer is a breast cancer. In some embodiments, the cancer is a colon cancer. In some embodiments, the cancer is a pancreatic cancer. In some embodiments, the cancer is a head and neck squamous cell carcinomas. In some embodiments, the cancer is a melanoma.

In another aspect, provided herein is a method of treating an autoimmune disease or disorder in a subject in need thereof, the method comprising administering a therapeutically effective amount of a compound described herein, or a pharmaceutically acceptable salt, or stereoisomer thereof, or a pharmaceutical composition described herein.

### Dosing

In certain embodiments, the compositions containing the compound(s) described herein are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

### Pharmaceutical Compositions/Formulations

The compounds described herein are administered to a subject in need thereof, either alone or in combination with pharmaceutically acceptable carriers, excipients, or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### EXAMPLES

### Preparation of Intermediate 1 and Intermediate 2

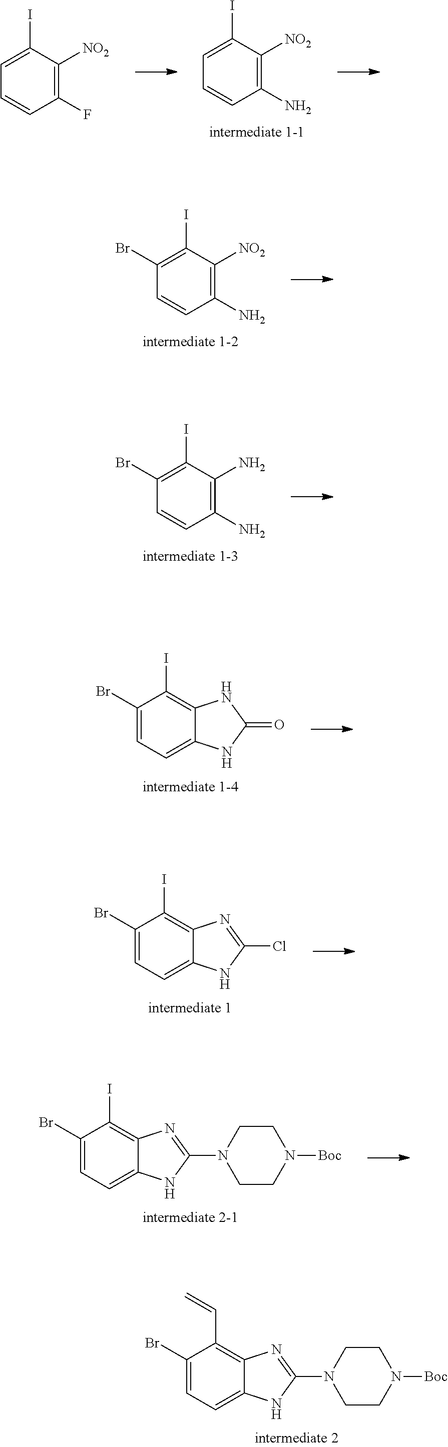

Step 1: To a solution of 1-fluoro-3-iodo-2-nitrobenzene (20.00 g, 74.91 mmol) in MeOH (50 mL) was added NH₃·H₂O (100 mL, 25%) at 20° C. After addition, the reaction was heated to 110° C. and stirred for 48 hrs. After cooling to room temperature, the resulting mixture was concentrated to dryness under reduced pressure (remove MeOH) to give a residue. Water (500 mL) was added and extracted with EtOAc (300 mL×3). The combined organic layers were washed with brine (100 mL), dried over Na₂SO₄, filtered, and concentrated under reduced pressure to afford the desired product. LC-MS (ESI+): Rt=2.390 min; m/z 265.0 [M+H]⁺.

Step 2: To a solution of intermediate 1-1 (20.00 g, 75.75 mmol) in DMF (200 mL) was added NBS (11.50 g, 64.30 mmol) at 0° C. After addition, the mixture was stirred at 0° C. for 10 mins and then warmed to 20° C. for 2 hrs. The reaction mixture was quenched with water (1000 mL) and filtered. The filter cake was washed with water (100 mL×3) and concentrated to dryness under reduced pressure to afford the desired product. LC-MS (ESI+): Rt=2.523 min; m/z 342.8 [M+H]⁺.

Step 3: To a solution of intermediate 1-2 (24.00 g, 69.99 mmol) in EtOH (300 mL) and water (60 mL) were added Fe (39.10 g, 69.91 mmol) and NH₄Cl (37.40 g, 699.91 mmol). After addition, the mixture was stirred at 50° C. for 2 hrs. After cooling to room temperature, the suspension was filtered. The filter cake was washed with EtOH (200 mL×3). The combined filtrates were concentrated to dryness under reduced pressure to give a residue, which was adjusted pH to 8 with saturated sodium bicarbonate (500 mL) and then extracted with EtOAc (300 mL×3). The combined organic phases were concentrated to dryness under reduced pressure to afford the desired product. LC-MS (ESI+): Rt=1.959 min; m/z 312.8 [M+H]⁺.

Step 4: To a solution of intermediate 1-3 (20.00 g, 63.90 mmol) in toluene (100 mL) was added CDI (15.60 g, 96.2 mmol) at 20° C. After addition, the mixture was stirred at 80° C. for 2 hrs. After cooling to room temperature, water (500 mL) was added and the mixture was filtered. The filter cake was washed with EtOH (100 mL×3) and concentrated to dryness under reduced pressure to afford the desired product. LC-MS (ESI+): Rt=1.843 min; m/z 338.8 [M+H]⁺.

Step 5: To a solution of intermediate 1-4 (18.00 g, 53.10 mmol) in POCl₃ (100 mL) was added DMF (1 mL, 14.75 mmol) at 20° C. After addition, the reaction was stirred at 100° C. for 12 hrs. After cooling to room temperature, the resulting mixture was concentrated to dryness under reduced pressure (remove POCl₃) to give a residue, which was diluted with H₂O (500 mL) and extracted with EtOAc (300 mL×2). Then the combined organic layers were washed with brine (200 mL×2), dried over Na₂SO₄, filtered, and concentrated under reduced pressure to afford the desired product (intermediate 1). LC-MS (ESI+): Rt=2.298 min; m/z 356.7 [M+H]⁺. ¹H NMR: (400 MHz, DMSO-d₆) δ 7.53 (d, J=8.0 Hz, 1H), 7.44 (d, J=8.0 Hz, 1H).

Step 6: To a mixture of intermediate 1 (1200 mg, 3.56 mmol) in NMP (30 mL) was added 2-methylpropan-2-yl piperazine-1-carboxylate (1200 mg, 6.44 mmol) at 20° C. After addition, the mixture was stirred at 130° C. for 4 hrs under N₂. After cooling to 20° C., the resulting mixture was diluted with water (100 mL) and then extracted with EtOAc (100 mL×2). The combined organic layers were washed with water (100 mL×2), brine (100 mL×2), filtrated and concentrated under reduced pressure to give a residue, which was triturated with MeOH (5 mL) at 20° C. for 20 mins to afford the desired product. LC-MS (ESI+): Rt=1.650 min; m/z 506.8 [M+H]⁺.

Step 7: To a solution of intermediate 2-1 (900 mg, 1.77 mmol), Na₂CO₃ (564.30 mg, 5.32 mmol) and potassium trifluoro (vinyl) borate (285.3 mg, 2.13 mmol) in dioxane (20 mL) and H₂O (4 mL) was added Pd(dppf)Cl₂ (129.80 mg, 0.18 mmol) under N₂. After addition, the mixture was purged with N₂ for 2 mins and then stirred at 80° C. for 12 hrs under N₂. After cooling to room temperature, the resulting mixture was poured to water (40 mL) and extracted with EtOAc (100 mL×3). The combined organic layers were washed with brine (100 mL×2), dried over Na₂SO₄, filtered, and concentrated under reduced pressure to give a residue, which was purified by column chromatography (SiO₂, PE:EtOAc:THF=10:1:1 to 1:1:1) to afford the desired product (intermediate 2). LC-MS (ESI+): Rt=1.416 min; m/z 407.2 [M+H]⁺.

### Preparation of Intermediate 3

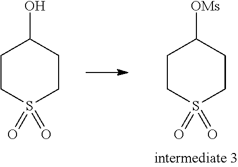 [SMILES: O=S1(=O)CCC(O)CC1 ; CS(=O)(=O)OC1CCS(=O)(=O)CC1]

To a mixture of 4-hydroxytetrahydro-2H-thiopyran 1,1-dioxide (2.00 g, 13.32 mmol) and TEA (2.59 mL, 19.97 mmol) in DCM (20 mL) was added methanesulfonyl chloride (1.24 mL, 15.98 mmol) at 0° C. After addition, the mixture was stirred at 20° C. for 3 hrs. The resulting mixture was diluted with DCM (100 mL) and washed with water (100 mL×2), brine (50 mL×2), dried over Na₂SO₄ and concentrated to afford the desired product (intermediate 3). ¹H NMR (400 MHz, CDCl₃) δ 5.06-5.01 (m, 1H), 3.37-3.28 (m, 2H), 3.10 (s, 3H), 3.06-2.98 (m, 2H), 2.56-2.39 (m, 4H).

### Preparation of Intermediate 4

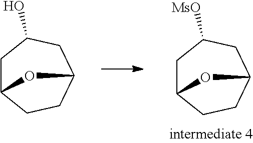 [SMILES: O[C@H]1C[C@H]2CC[C@@H](C1)O2 ; CS(=O)(=O)O[C@H]1C[C@H]2CC[C@@H](C1)O2]

To a solution of (3-endo)-8-oxabicyclo[3.2.1]octan-3-ol (5.00 g, 39.01 mmol) and TEA (8.13 mL, 58.52 mmol) in DCM (50 mL) was added methanesulfonyl chloride (3.62 mL, 46.81 mmol) at 0° C. After addition, the mixture was stirred at 20° C. for 2 hrs. The reaction mixture was poured into ice-water (100 mL) and extracted with DCM (50 mL×2). The combined organic layers were washed with brine (100 mL), dried over Na₂SO₄, and concentrated under reduced pressure to afford the desired product (intermediate 4). ¹H NMR (400 MHz, CDCl₃) δ 5.03-4.98 (m, 1H), 4.42-4.37 (m, 2H), 3.01 (s, 3H), 2.27-2.12 (m, 4H), 2.02-1.91 (m, 4H).

### Preparation of Intermediate 5

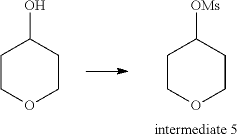 [SMILES: OC1CCOCC1 ; CS(=O)(=O)OC1CCOCC1]

To a solution of tetrahydro-2H-pyran-4-ol (180.00 g, 1.76 mol) in DCM (1200 mL) were added TEA (319.6 L, 2.46 mol) and MsCl (163.6 mL, 2.11 mol) at 0° C. After addition, the reaction mixture was stirred at 10° C. for 3 hrs. Then the resulting mixture was quenched with water (2000 mL) and extracted with DCM (1000 mL×2). The combined organic layers were washed with brine (2000 mL), dried over Na₂SO₄, filtered, and concentrated under reduced pressure to afford the desired product (intermediate 5), which was used for next step directly without further purification. ¹H NMR (400 MHz, CDCl₃) δ 4.94-4.86 (m, 1H), 3.99-3.89 (m, 2H), 3.59-3.51 (m, 2H), 3.04 (s, 3H), 2.09-2.01 (m, 2H), 1.94-1.83 (m, 2H).

### Preparation of Intermediate 6 and Intermediate 7

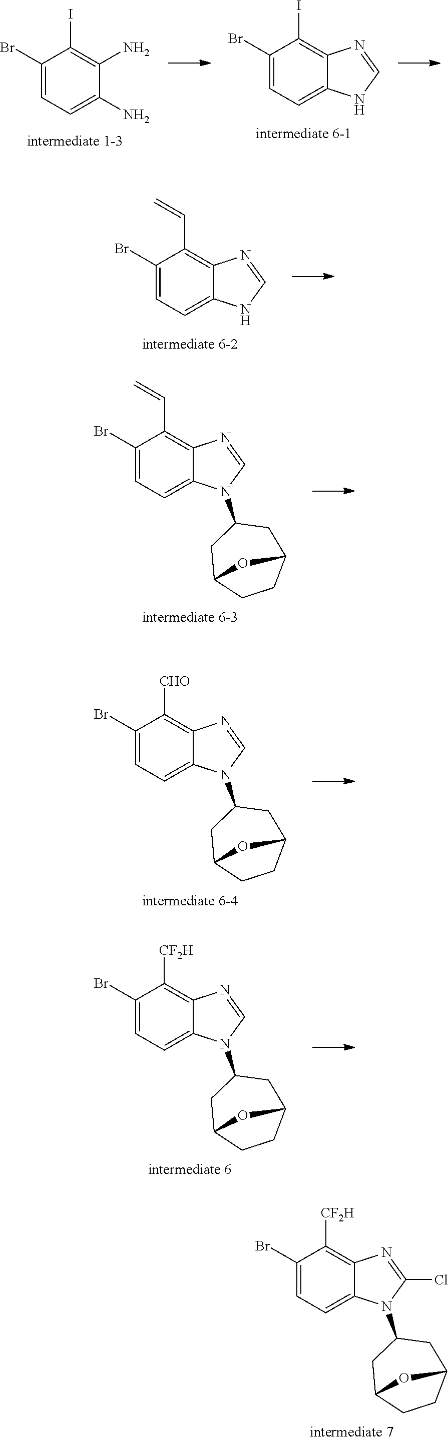

Step 1: To a solution of intermediate 1-3 (21.00 g, 67.11 mmol) in MeOH (100 mL) was added trimethoxymethane (21.40 g, 201.34 mmol) and TsOH (1.20 g, 6.71 mmol) at 20° C. After addition, the reaction mixture was stirred at 20° C. for 12 hrs. The reaction mixture was filtered. The filter cake was washed with MeOH (20 mL×3) and concentrated to dryness under reduced pressure to afford the desired product. LC-MS (ESI+): Rt=1.371 min; m/z 322.6 [M+H]⁺.

Step 2: To a solution of intermediate 6-1 (4.00 g, 12.39 mmol), Na₂CO₃ (3.90 g, 37.16 mmol) and potassium vinyltrifluoroborate (5.00 g, 37.16 mmol) in 1,4-dioxane (50 mL) and water (10 mL) was added Pd(dppf)Cl₂ (0.90 g, 0.12 mmol) at 20° C. After addition, the mixture was purged with N₂ for 2 mins and then stirred at 100° C. for 12 hrs under N₂. After cooling to 20° C., the reaction solution was poured into water (50 mL) and extracted with EtOAc (50 mL×3). The combined organic layers were washed with water (100 mL×2), brine (100 mL×2), dried over Na₂SO₄, filtered, and concentrated under reduced pressure to give a residue, which was purified by column chromatography (SiO₂, PE:EtOAc=10:1 to 3:1) to afford the desired product. LC-MS (ESI+): Rt=1.265 min; m/z 223.0 [M+H]⁺.

Step 3: To a mixture of intermediate 6-2 (1.60 g, 7.17 mmol), Cs₂CO₃ (4.67 g, 14.34 mmol) and tetrabutylammonium iodide (264.9 mg, 0.72 mmol) in NMP (10 mL) was added intermediate 4 (2.95 g, 14.34 mmol) at 20° C. After addition, the mixture was stirred at 135° C. for 12 hrs. After cooling to 20° C., the reaction solution was poured into ice water (50 mL) and extracted with EtOAc (50 mL×3). The combined organic layers were washed with water (100 mL×2), brine (100 mL×2), dried over Na₂SO₄, filtered, and concentrated under reduced pressure to give a residue, which was purified by column chromatography (SiO₂, PE:EtOAc=10:1 to 1:1) to afford the desired product. LC-MS (ESI+): Rt=1.633 min; m/z 333.0 [M+H]⁺.

Step 4: To a solution of intermediate 6-3 (450.0 mg, 1.35 mmol) in THF (10 mL) and water (10 mL) were added NaIO₄ (866.6 mg, 4.05 mmol) and K₂OsO₄·2H₂O (49.0 mg, 0.14 mmol) at 0° C. After addition, the mixture was stirred at 20° C. for 30 min. The reaction solution was poured into water (50 mL) and extracted with EtOAc (30 mL×3). The combined organic layers were washed with water (30 mL×2), brine (30 mL×2), dried over Na₂SO₄, filtered, and concentrated under reduced pressure to afford the desired product. LC-MS (ESI+): Rt=1.259 min; m/z 334.9 [M+H]⁺.

Step 5: To a solution of intermediate 6-4 (460 mg, 1.37 mmol) in DCM (5 mL) was added DAST (1.11 g, 6.86 mmol) dropwise at 0° C. After addition, the mixture was stirred at 50° C. for 30 min under N₂. After cooling to 20° C., the resulting mixture was quenched with water (50 mL) and extracted with DCM (50 mL×2). The combined organic layers were washed with brine (50 mL×2), dried over Na₂SO₄, filtered, and concentrated under reduced pressure to give a residue, which was purified by prep-TLC (SiO₂, PE:EtOAc=2:1) to afford the desired product (intermediate 6). LC-MS (ESI+): Rt=1.673 min; m/z 356.9 [M+H]⁺.

Step 6: To a solution of intermediate 6 (130.0 mg, 0.36 mmol) in THF (10 mL) was added LDA (0.36 mL, 2M) dropwise at −78° C. over 5 min. After addition, the mixture was stirred at −78° C. for 1 hr, and then perchloroethane (172.3 mg, 0.72 mmol) in THF (1 mL) was added dropwise at −78° C. The resulting mixture was stirred at −78° C. for 2 hr under N₂. The resulting mixture was quenched with water (30 mL) and extracted with EtOAc (30 mL×2). The organic layers were washed with water (50 mL×2), brine (50 mL×2), dried over Na₂SO₄, filtered, and concentrated under reduced pressure to give a residue, which was purified by prep-TLC (SiO₂, PE:EtOAc=1:1) to afford intermediate 7. LC-MS (ESI+): Rt=2.652 min; m/z 390.8 [M+H]⁺. ¹H NMR (400 MHz, DMSO-d₆) δ 7.87-7.85 (d, J=8.0 Hz, 1H), 7.64-7.62 (d, J=8.0 Hz, 1H), 7.60-7.34 (t, J=12.0 Hz, 1H), 4.97-4.90 (m, 1H), 4.53 (s, 2H), 2.47-2.40 (m, 2H), 1.95 (m, 4H), 1.87-1.82 (m, 2H).

### Example 1

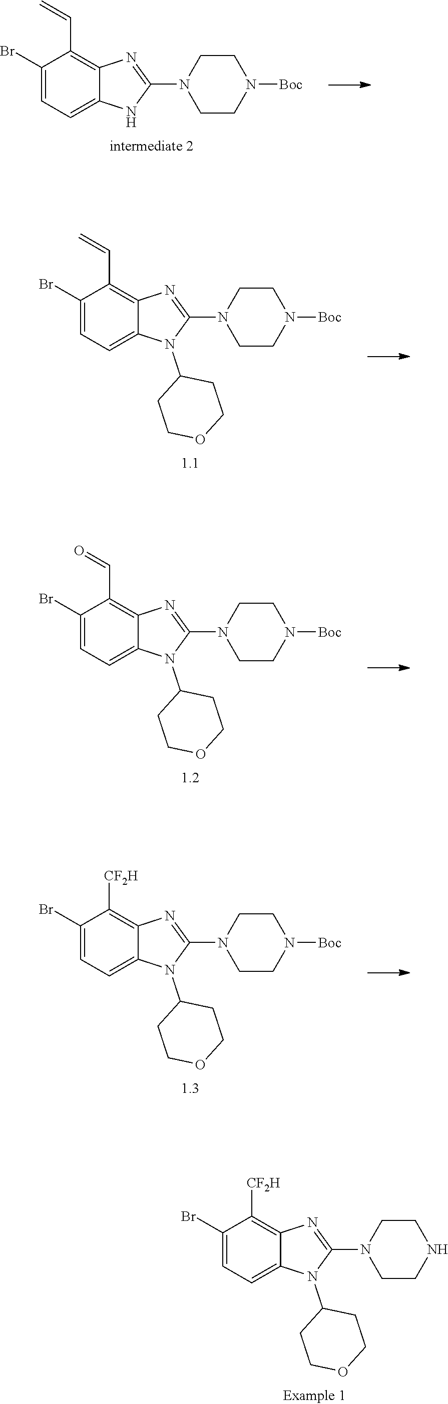 [SMILES: C=Cc1c(Br)ccc2[nH]c(N3CCN(C(=O)OC(C)(C)C)CC3)nc12 ; C=Cc1c(Br)ccc2c1nc(N1CCN(C(=O)OC(C)(C)C)CC1)n2C1CCOCC1 ; CC(C)(C)OC(=O)N1CCN(c2nc3c(C=O)c(Br)ccc3n2C2CCOCC2)CC1 ; CC(C)(C)OC(=O)N1CCN(c2nc3c(C(F)F)c(Br)ccc3n2C2CCOCC2)CC1 ; FC(F)c1c(Br)ccc2c1nc(N1CCNCC1)n2C1CCOCC1]

Step 1: To a mixture of intermediate 2 (250.0 mg, 0.61 mmol), Cs₂CO₃ (599.9 mg, 1.84 mmol) and TBAI (68.00 mg, 0.18 mmol) in NMP (5 mL) was added intermediate 5 (331.8 mg, 1.84 mmol) at 20° C. After addition, the mixture was stirred at 130° C. for 4 hrs. After cooling to 20° C., the resulting mixture was diluted with water (10 mL) and extracted with EtOAc (10 mL×2). The combined organic layers were washed with H₂O (10 mL×3), brine (10 mL×2), dried over Na₂SO₄, filtered, and concentrated under reduced pressure to give a residue, which was purified by prep-TLC (SiO₂, PE:EtOAc=1:1) to afford the desired product. LC-MS (ESI+): Rt=3.295 min; m/z 490.9 [M+H]⁺.

Step 2: To a solution of 1.1 (150.0 mg, 0.31 mmol) in THF (10 mL) and H₂O (10 mL) were added NaIO₄ (195.1 mg, 0.92 mmol) and K₂OsO₄·2H₂O (9.30 mg, 0.030 mmol) at 0° C. After addition, the reaction was stirred at 20° C. for 2 hrs. To the resulting mixture was added water (50 mL) and extracted with EtOAc (50 mL×2). The combined organic layers were washed with brine (50 mL×2), dried over Na₂SO₄, filtered, and concentrated under reduced pressure to afford the desired product. LC-MS (ESI+): Rt=2.416 min; m/z 492.8 [M+H]⁺.

Step 3: To a solution of 1.2 (50.0 mg, 0.10 mmol) in DCM (10 mL) was added DAST (0.20 mL, 1.52 mmol) at 0° C. After addition, the mixture was stirred at 50° C. for 2 hrs under N₂. After cooling to room temperature, the resulting mixture was diluted with H₂O (30 mL) and extracted with DCM (50 mL×2). The combined organic layers were washed with brine (50 mL×2), dried over Na₂SO₄, filtered, and concentrated under reduced pressure to give a residue, which was purified by prep-TLC (SiO₂, PE:EtOAc=3:1) to afford the desired product. LC-MS (ESI+): Rt=3.009 min; m/z 514.8 [M+H]⁺.

Step 4: To a solution of 1.3 (60.0 mg, 0.12 mmol) in EtOAc (2 mL) was added HCl/1,4-dioxane (4M, 3 mL) at 0° C. After addition, the mixture was stirred at 20° C. for 1 hr. The reaction mixture was concentrated under reduced pressure to give a residue, which was purified by prep-HPLC (Column: Waters Xbridge Prep OBD C18 20× 250 mm×10 μm; Mobile phase: from 20% to 50% ACN in water [0.1% NH₃·H₂O]; Wavelength: 220 nm; Flow rate: 20 mL/min) to afford the desired product. LC-MS (ESI+): Rt=1.084 min; m/z 415.1 [M+H]⁺. ¹H NMR (400 MHz, methanol-d₄) δ 7.65 (d, J=8.0 Hz, 1H), 7.58-7.31 (m, 2H), 4.59-4.51 (m, 1H), 4.17-4.13 (m, 2H), 3.66-3.61 (m, 2H), 3.38-3.36 (m, 4H), 3.22-3.19 (m, 4H), 2.57-2.54 (m, 2H), 1.88-1.82 (m, 2H).

### Example 2

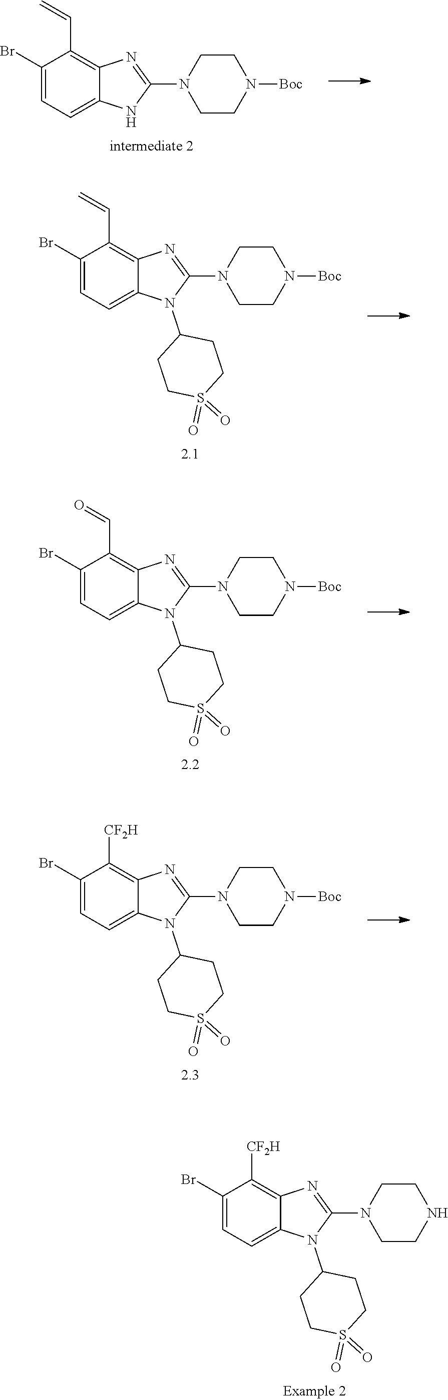 [SMILES: C=Cc1c(Br)ccc2[nH]c(N3CCN(C(=O)OC(C)(C)C)CC3)nc12 ; C=Cc1c(Br)ccc2c1nc(N1CCN(C(=O)OC(C)(C)C)CC1)n2C1CCS(=O)(=O)CC1 ; CC(C)(C)OC(=O)N1CCN(c2nc3c(C=O)c(Br)ccc3n2C2CCS(=O)(=O)CC2)CC1 ; CC(C)(C)OC(=O)N1CCN(c2nc3c(C(F)F)c(Br)ccc3n2C2CCS(=O)(=O)CC2)CC1 ; O=S1(=O)CCC(n2c(N3CCNCC3)nc3c(C(F)F)c(Br)ccc32)CC1]

Step 1: To a mixture of intermediate 2 (400 mg, 0.98 mmol), Cs₂CO₃ (959.9 mg, 2.95 mmol) and TBAI (36.30 mg, 0.098 mmol) in NMP (5 mL) was added intermediate 3 (672.6 mg, 2.95 mmol) at 20° C. After addition, the mixture was stirred at 130° C. for 4 hrs. After cooling to 20° C., the resulting mixture was diluted with water 10 mL and extracted with EtOAc (10 mL×2). The combined organic layers were washed with H₂O (10 mL×3), brine (10 mL×2), dried over Na₂SO₄, filtered, and concentrated under reduced pressure to give a residue, which was purified by column chromatography (SiO₂, PE:EtOAc:THF=10:1:1 to DCM:MeOH=100:1) to afford the desired product. LC-MS (ESI+): Rt=3.137 min; m/z 539.2 [M+H]⁺.

Step 2: To a solution of 2.1 (330.0 mg, 0.61 mmol) in THF (10 mL) and H₂O (10 mL) were added NaIO₄ (390.9 mg, 1.84 mmol) and K₂OsO₄·2H₂O (15.50 mg, 0.061 mmol) at 0° C. After addition, the reaction was stirred at 20° C. for 2 hrs. The resulting mixture was poured to water (50 mL) and then extracted with EtOAc (50 mL×2). Then the combined organic layers were washed with brine (50 mL×2), dried over Na₂SO₄, filtered, and concentrated under reduced pressure to afford the desired product. LC-MS (ESI+): Rt=3.130 min; m/z 541.2 [M+H]⁺.

Step 3: To a solution of 2.2 (280 mg, 0.52 mmol) in DCM (10 mL) was added DAST (0.34 mL, 2.59 mmol) at 0° C. After addition, the mixture was stirred at 50° C. for 2 hrs under N₂. After cooling to room temperature, the resulting mixture was diluted with H₂O (30 mL) and extracted with DCM (50 mL×2). Then the combined organic layers were washed with brine (30 mL×2), dried over Na₂SO₄, filtered, and concentrated under reduced pressure to give a residue, which was purified by prep-TLC (SiO₂, PE:EtOAc=5:2) to afford the desired product. LC-MS (ESI+): Rt=2.786 min; m/z 563.2 [M+H]⁺.

Step 4: To a solution of 2.3 (110 mg, 0.24 mmol) in EtOAc (1 mL) was added HCl/1,4-dioxane (4M, 5 mL) dropwise at 0° C. After addition, the mixture was stirred at 20° C. for 1 hr. The resulting mixture was concentrated under reduced pressure to give a residue, which was purified by prep-HPLC (Column: Waters Xbridge Prep OBD C18 20× 250 mm×10 μm; Mobile phase: from 25% to 50% ACN in water [0.1% NH₃·H₂O]; Wavelength: 220 nm; Flow rate: 20 mL/min.) to afford the desired product. LC-MS (ESI+): Rt=1.285 min; m/z 463.1 [M+H]⁺. ¹H NMR (400 MHz, methanol-d₄) δ 7.60-7.29 (m, 3H), 4.70-4.64 (m, 1H), 3.59-3.49 (m, 2H), 3.28-3.22 (m, 4H), 3.21-3.13 (m, 2H), 3.09-3.05 (m, 4H), 3.05-2.95 (m, 2H), 2.30-2.18 (m, 2H).

The following compound was prepared using a synthesis method similar to that described in Example 3.

| | LC-MS (ESI+) | |
|---|---|---|
| Example | [M + H]⁺ | ¹H NMR (400 MHz) |
| 13 | 462.0 | DMSO-d₆ δ 7.61-7.34 (m, 3H), 6.13 (s, 1H), 4.70-4.50 (m, 1H), |
| | | 3.81 (s, 1H), 3.55-3.51 (m, 4H), 3.25-3.06 (m, 6H), 3.00-2.81 (m, |
| | | 4H), 2.15-2.07 (m, 2H). |

### Example 3

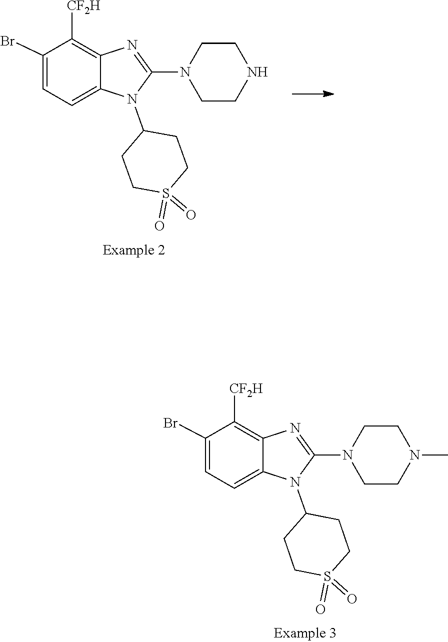 [SMILES: O=S1(=O)CCC(n2c(N3CCNCC3)nc3c(C(F)F)c(Br)ccc32)CC1 ; CN1CCN(c2nc3c(C(F)F)c(Br)ccc3n2C2CCS(=O)(=O)CC2)CC1]

To a solution of example 2 (22.0 mg, 0.047 mmol) in MeOH (1 mL) were added AcOH (8.50 mg, 0.14 mmol), NaBH₃CN (4.50 mg, 0.071 mmol) and formaldehyde (1.40 mg, 0.047 mmol) at 0° C. After addition, the mixture was stirred at 20° C. for 2 hrs. The reaction mixture was concentrated under reduced pressure to give a residue. Then, to the mixture was added water (30 mL) and extracted with EtOAc (20 mL×3). The combined organic phases were concentrated to dryness under reduced pressure to afford a residue, which was purified by prep-HPLC (Column: Waters Xbridge Prep OBD C18 20×250 mm×10 μm; Mobile phase: from 25% to 50% ACN in water [0.1% NH₃·H₂O]; Wavelength: 220 nm; Flow rate: 20 mL/min.) to afford the desired product. LC-MS (ESI+): Rt=1.298 min; m/z 477.0 [M+H]⁺. ¹H NMR (400 MHz, methanol-d₄) δ 7.66-7.31 (m, 3H), 4.73-4.63 (m, 1H), 3.60-3.49 (m, 2H), 3.40-3.42 (m, 4H), 3.25-3.15 (m, 2H), 3.11-2.97 (m, 2H), 2.90-2.65 (m, 4H), 2.44 (s, 3H), 2.32-2.18 (m, 2H).

The following compounds were prepared using a synthesis method similar to that described in Example 3.

| | LC-MS (ESI+) | |
|---|---|---|
| Example | [M + H]⁺ | ¹H NMR (400 MHz) |
| 7 | 429.2 | methanol-d₄ δ 7.60 (d, J = 8.0 Hz, 1H), 7.42 (t, J = 52.0 Hz, 1H), 7.40 |
| | | (d, J = 8.0 Hz, 1H), 4.51-4.47 (m, 1H), 4.15-4.11 (m, 2H), 3.63- |
| | | 3.56 (m, 2H), 3.35-3.33 (m, 4H), 2.72-2.69 (m, 4H), 2.54-2.49 (m, |
| | | 2H), 2.40 (s, 3H), 1.83-1.79 (m, 2H). |
| 21 | 511.0 | methanol-d₄ δ 7.58-7.30 (m, 3H), 4.78-4.72 (m, 1H), 4.61 (s, 2H), |
| | | 4.42-4.36 (m, 1H), 3.66-3.33 (m, 4H), 3.13-3.10 (m, 1H), 2.70- |
| | | 2.53 (m, 6H), 2.34-2.29 (m, 2H), 2.15-2.09 (m, 4H), 1.96-1.90 (m, |
| | | 2H), 1.82-1.78 (m, 2H). |
| 22 | 510.8 | methanol-d₄ δ 7.55-7.30 (m, 3H), 4.79-4.70 (m, 1H), 4.61 (s, 2H), |
| | | 4.04-3.96 (m, 1H), 3.35-3.32 (m, 4H), 2.63-2.40 (m, 9H), 2.16- |
| | | 2.06 (m, 2H), 1.95-1.90 (m, 2H), 1.88-1.77 (m, 4H). |
| 27 | 455.0 | methanol-d₄ δ 7.58-7.30 (m, 3H), 4.79-4.73 (m, 1H), 4.61 (s, 2H), |
| | | 3.35-3.32 (m, 4H), 2.72-2.69 (m, 4H), 2.60-2.53 (m, 2H), 2.41 (s, |
| | | 3H), 2.15-2.12 (m, 2H), 1.96-1.91 (m, 2H), 1.82-1.77 (m, 2H). |

### Example 4

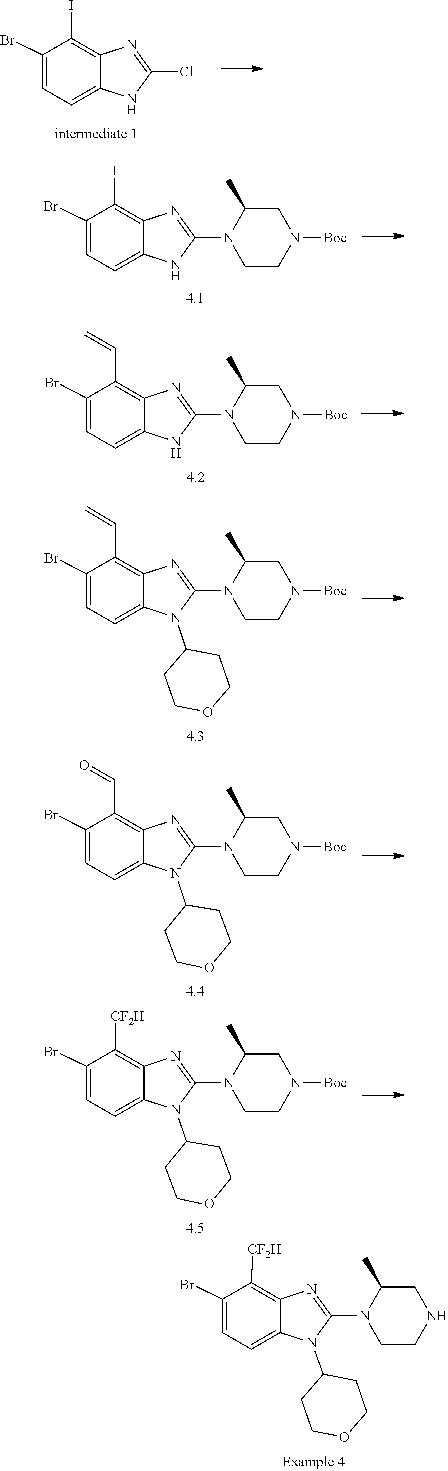

Step 1: To a mixture of intermediate 1 (2.00 g, 5.60 mmol) in NMP (20 mL) were added DIEA (3.89 mL, 22.45 mmol) and tert-butyl(S)-3-methylpiperazine-1-carboxylate (3.30 g, 16.48 mmol) at 20° C. After addition, the mixture was stirred at 130° C. for 8 hrs under N₂. After cooling to 20° C. The resulting mixture was quenched with water (100 mL) and extracted with EtOAc (100 mL×2). The combined organic layers were washed with water (100 mL×2), brine (150 mL×2), dried over Na₂SO₄, filtrate and concentrated under reduced pressure to give a residue, which was triturated with MeOH (10 mL) at 20° C. for 20 mins and filtered, the filter cake was dried to afford the desired product. LC-MS (ESI+): Rt=1.350 min; m/z 520.8 [M+H]⁺.

Step 2: To a solution of 4.1 (1.00 g, 1.92 mmol), Na₂CO₃ (0.60 g, 5.76 mmol) and potassium trifluoro (vinyl) borate (0.80 g, 5.76 mmol) in dioxane (50 mL) and H₂O (10 mL) was added Pd(dppf)Cl₂ (0.10 g, 0.19 mmol). After addition, the mixture was purged with N₂ for 3 mins and then stirred at 70° C. for 12 hrs under N₂. After cooling to room temperature, the reaction mixture was poured to water (100 mL) and extracted with EtOAc (50 mL×3). The combined organic layers were washed with brine (100 mL×2), dried over Na₂SO₄, filtered, and concentrated under reduced pressure to give a residue, which was purified by column chromatography (SiO₂, PE:EtOAc=10:1 to 3:1) to afford the desired product. LC-MS (ESI+): Rt=2.043 min; m/z 420.8 [M+H]⁺.

Step 3: To a mixture of 4.2 (150.0 mg, 0.36 mmol), Cs₂CO₃ (578.6 mg, 1.78 mmol) and TBAI (26.3 mg, 0.071 mmol) in NMP (3 mL) was added intermediate 5 (320.8 mg, 1.78 mmol) at 20° C. After addition, the mixture was stirred at 130° C. for 4 hrs. After cooled to 20° C., the resulting mixture was diluted with water (30 mL) and extracted with EtOAc (10 mL×2). The combined organic layers were washed with brine (20 mL), dried over Na₂SO₄, filtered, and concentrated under reduced pressure to give a residue, which was purified by column chromatography (SiO₂, PE:EtOAc:THF=10:1:1 to 10:3:1) to afford the desired product. LC-MS (ESI+): Rt=3.756 min; m/z 505.1 [M+H]⁺.

Step 4: To a solution of 4.3 (100.0 mg, 0.20 mmol) in THF (3 mL) and H₂O (3 mL) were added NaIO₄ (168.5 mg, 1.791 mmol) and K₂OsO₄·2H₂O (7.30 mg, 0.02 mmol) at 0° C. After addition, the reaction was stirred at 20° C. for 2 hrs. The reaction mixture was poured to water (50 mL) extracted with EtOAc (50 mL×2), the combined organic layers were washed with brine (10 mL×2), dried over Na₂SO₄, filtered, and concentrated under reduced pressure to afford the desired product. LC-MS (ESI+): Rt=2.889 min; m/z 506.8 [M+H]⁺.

Step 5: To a solution of 4.4 (100 mg, 0.20 mmol) in DCM (5 mL) was added DAST (46.0 mg, 0.29 mmol) at 0° C. After addition, the mixture was stirred at 50° C. for 2 hrs under N₂. After cooling to room temperature, the reaction mixture was diluted with H₂O (30 mL) and extracted with DCM (20 mL×2). Then the combined organic layers were washed with brine (30 mL×2), dried over Na₂SO₄, filtered, and concentrated under reduced pressure to give a residue, which was purified by prep-TLC (SiO₂, PE:EtOAc=5:2) to afford the desired product. LC-MS (ESI+): Rt=3.279 min; m/z 528.8 [M+H]⁺.

Step 6: To a solution of 4.5 (50.0 mg, 0.094 mmol) in EtOAc (2 mL) was added HCl/1,4-dioxane (4M, 2 mL) at 20° C. After addition, the mixture was stirred at 20° C. for 4 hrs. The reaction mixture was concentrated under reduced pressure to give a residue, which was purified by prep-HPLC (Column: Welch Xtimate C18 150×30 mm×5 μm; mobile phase: [water (NH₃·H₂O+NH₄HCO₃)-ACN]; B %: 20%-80%) to afford the desired product. LC-MS (ESI+): Rt=1.378 min; m/z 431.1 [M+H]⁺. ¹H NMR (400 MHz, methanol-d₄) δ 7.69 (d, J=8.0 Hz, 1H), 7.47 (d, J=8.0 Hz, 1H), 7.43 (t, J=54.0 Hz, 1H), 4.85-4.75 (m, 1H), 4.17-4.09 (m, 2H), 3.66-3.58 (m, 2H), 3.55-3.46 (m, 1H), 3.18-3.01 (m, 5H), 2.76-2.66 (m, 1H), 2.61-2.46 (m, 2H), 1.91-1.84 (m, 1H), 1.673-1.66 (m, 1H), 0.97 (d, J=4.0 Hz, 3H).

The following compounds were prepared using a synthesis method similar to that described in Example 4.

| | LC-MS (ESI+) | |
|---|---|---|
| Example | [M + H]⁺ | ¹H NMR (400 MHz) |
| 5 | 477.0 | methanol-d₄) δ 7.66 (d, J = 8.0 Hz, 1H), 7.53 (d, J = 8.0 Hz, 1H), 7.42 |
| | | (t, J = 54.0 Hz, 1H), 5.04-4.94 (m, 1H), 3.60-3.49 (m, 3H), 3.24- |
| | | 3.13 (m, 6H), 3.13-3.02 (m, 2H), 3.01-2.92 (m, 1H), 2.88-2.78 (m, |
| | | 1H), 2.34-2.20 (m, 1H), 2.17-2.07 (m, 1H), 0.99 (d, J = 4.0 Hz, 3H). |
| 23 | 440.9 | methanol-d₄ δ 7.40 (t, J = 54.0 Hz, 1H), 7.47-7.45 (d, J = 8.0 Hz, |
| | | 1H), 7.33-7.31 (d, J = 8.0 Hz, 1H), 4.87-4.81 (m, 1H), 4.60 (s, 2H), |
| | | 3.88-3.81 (m, 2H), 3.75-3.66 (m, 2H), 3.46-3.42 (m, 1H), 2.65- |
| | | 2.57 (m, 2H), 2.30-2.23 (m, 1H), 2.13-1.76 (m, 7H). |

### Example 6

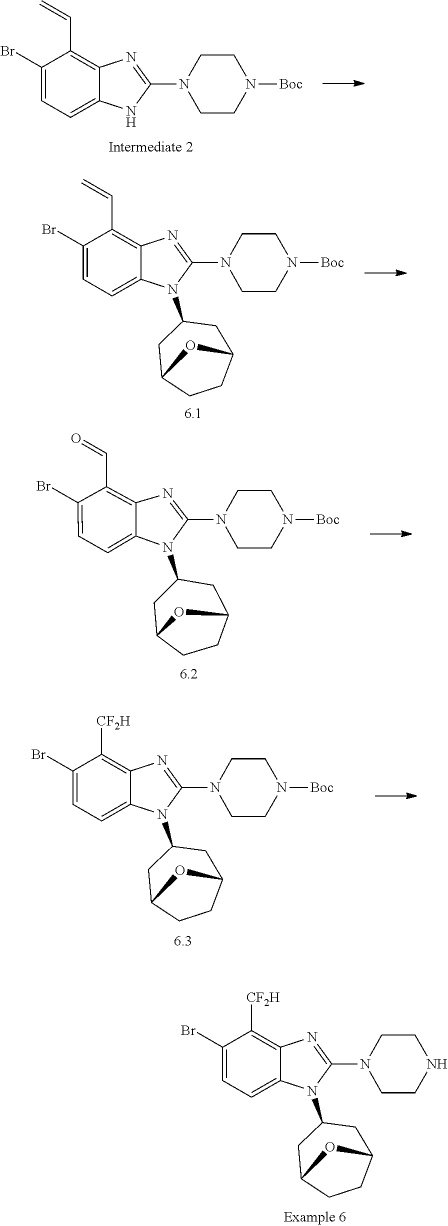 [SMILES: C=Cc1c(Br)ccc2[nH]c(N3CCN(C(=O)OC(C)(C)C)CC3)nc12 ; C=Cc1c(Br)ccc2c1nc(N1CCN(C(=O)OC(C)(C)C)CC1)n2[C@@H]1C[C@H]2CC[C@@H](C1)O2 ; CC(C)(C)OC(=O)N1CCN(c2nc3c(C=O)c(Br)ccc3n2[C@@H]2C[C@H]3CC[C@@H](C2)O3)CC1 ; CC(C)(C)OC(=O)N1CCN(c2nc3c(C(F)F)c(Br)ccc3n2[C@@H]2C[C@H]3CC[C@@H](C2)O3)CC1 ; FC(F)c1c(Br)ccc2c1nc(N1CCNCC1)n2[C@@H]1C[C@H]2CC[C@@H](C1)O2]

Step 1: To a mixture of intermediate 2 (438 mg, 1.08 mmol) and intermediate 4 (1.11 g, 5.38 mmol) in NMP (10 mL) were added Cs₂CO₃ (1751.8 mg, 5.38 mmol) and TBAI (39.7 mg, 0.11 mmol) at 25° C. After addition, the mixture was stirred at 130° C. for 4 hrs. After cooling to 25° C., the resulting mixture was quenched with water (10 mL) and extracted with EtOAc (30 mL×3). The combined organic layers were washed with H₂O (10 mL×3), brine (10 mL×2), dried over Na₂SO₄, filtered, and concentrated under reduced pressure to give a residue, which was purified by column chromatography (SiO₂, PE:EtOAc:THF=10:1:1 to 1:1:1) to afford the desired product. LC-MS (ESI+): Rt=3.631 min; m/z 517.0 [M+H]⁺.

Step 2: To a solution of 6.1 (150 mg, 0.29 mmol) in THF (5 mL) and H₂O (5 mL) were added NaIO₄ (186.0 mg, 0.87 mmol) and K₂OsO₄·2H₂O (7.4 mg, 0.02 mmol) at 0° C. After addition, the mixture was stirred at 25° C. for 30 mins. The residue was extracted with EtOAc (10 mL×2) and the combined organic layers were washed with water (10 mL×2), brine (20 mL×2), dried over Na₂SO₄, filtered, and concentrated under reduced pressure to afford the desired product. LC-MS (ESI+): Rt=2.769 min; m/z 519.0 [M+H]⁺.

Step 3: To a solution of 6.2 (186 mg, 0.36 mmol) in DCM (2 mL) was added DAST (0.24 mL, 1.79 mmol) dropwise at 0° C. After addition, the mixture was stirred at 50° C. for 2 hrs. After cooling to room temperature, the resulting mixture was put into ice water (10 mL) and extracted with DCM (5 mL×2). The combined organic layers were washed with water (2 mL×2), brine (10 mL×2), dried over Na₂SO₄, filtered, and concentrated under reduced pressure to afford the desired product. LC-MS (ESI+): Rt=3.289 min; m/z 540.8 [M+H]⁺.

Step 4: To a solution of 6.3 (150.0 mg, 0.28 mmol) in EtOAc (2 mL) was added HCl/dioxane (4M, 2 mL) dropwise at 0° C. After addition, the mixture was stirred at 25° C. for 30 mins. The reaction mixture was concentrated under reduced pressure to give a residue, which was purified by prep-HPLC (Column: Welch Xtimate C18 150×30 mm×5 μm; mobile phase: [water (NH₃·H₂O+NH₄HCO₃)-ACN]; B %: 20%-80%) to afford the desired product. LC-MS (ESI+): Rt=1.551 min; m/z 440.9 [M+H]⁺. ¹H NMR (400 MHz, methanol-d₄) δ 7.57-7.27 (m, 3H), 4.81-4.69 (m, 1H), 4.61-4.54 (m, 2H), 3.28-3.22 (m, 4H), 3.12-3.00 (m, 4H), 2.59-2.49 (m, 2H), 2.16-2.07 (m, 2H), 1.96-1.88 (m, 2H), 1.81-1.72 (m, 2H).

The following compounds were prepared using a synthesis method similar to that described in Example 6.

| | LC-MS (ESI+) | |
|---|---|---|
| Example | [M + H]⁺ | ¹H NMR (400 MHz) |
| 11 | 471.0 | methanol-d₄ δ 7.59-7.27 (m, 3H), 4.80-4.71 (m, 1H), 4.59 (s, 2H), |
| | | 3.69-3.67 (m, 2H), 3.55-3.46 (m, 2H), 3.35-3.30 (m, 2H), 3.30- |
| | | 3.28 (m, 2H), 3.14-3.10 (m, 1H), 2.60-2.53 (m, 2H), 2.13-2.08 (m, |
| | | 2H), 1.98-1.93 (m, 2H), 1.82-1.79 (m, 2H). |
| 12 | 454.7 | DMSO-d₆ δ 8.10 (s, 1H), 7.47 (t, J = 54.0 Hz, 1H), 7.56-7.54 (d, J = |
| | | 8.0 Hz, 1H), 7.45-7.43 (d, J = 8.0 Hz, 1H), 4.61-4.52 (m, 3H), 3.86 |
| | | (s, 2H), 3.46-3.43 (m, 2H), 3.41-3.37 (m, 2H), 2.43-2.34 (m, 2H), |
| | | 1.99-1.90 (m, 2H), 1.88-1.78 (m, 4H). |
| 18 | 397.1 | methanol-d₄ δ 7.64-7.62 (d, J = 8.0 Hz, 1H ), 7.45 (t, J = 54.0 Hz, |
| | | 1H), 7.27-7.25 (d, J = 8.0 Hz, 1H ), 4.86-4.74 (m, 1H), 4.62-4.59 |
| | | (m, 2H), 3.29-3.26 (m, 4H), 3.08-3.06 (m, 4H), 2.61-2.53 (m, 2H), |
| | | 2.15-2.12 (m, 2H), 1.97-1.92 (m, 2H), 1.82-1.78 (m, 2H). |
| 20 | 381.1 | methanol-d₄ δ 7.65-7.63 (m, 1H), 7.35 (t, J = 52.0 Hz, 1H), 7.06- |
| | | 7.01 (m, 1H), 4.82-4.76 (m, 1H), 4.63-4.59 (m, 2H), 3.28-3.25 (m, |
| | | 4H), 3.08-3.05 (m, 4H), 2.61-2.54 (m, 2H), 2.15-2.12 (m, 2H), |
| | | 1.97-1.92 (m, 2H), 1.82-1.78 (m, 2H). |

### Example 8

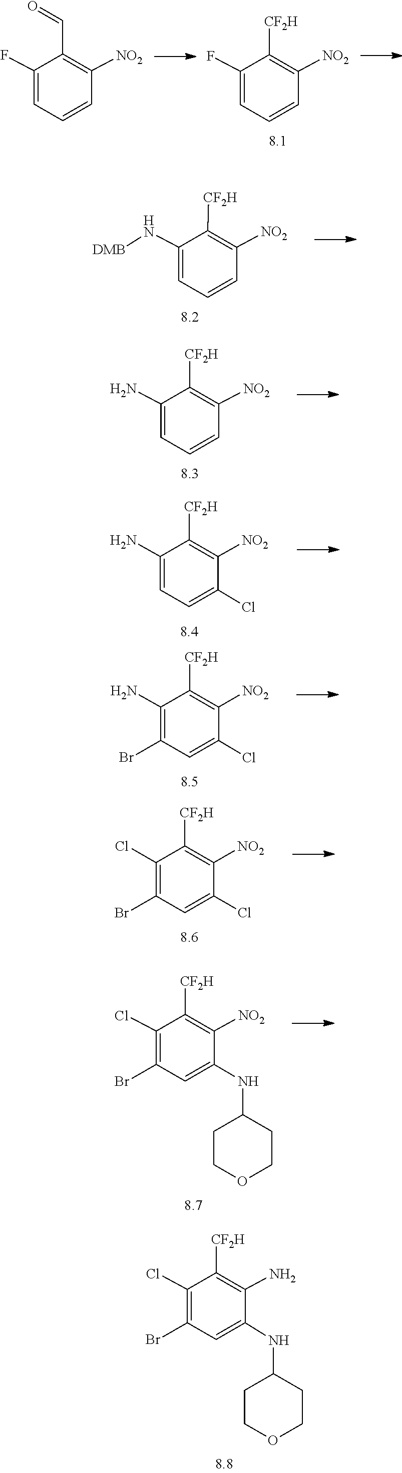

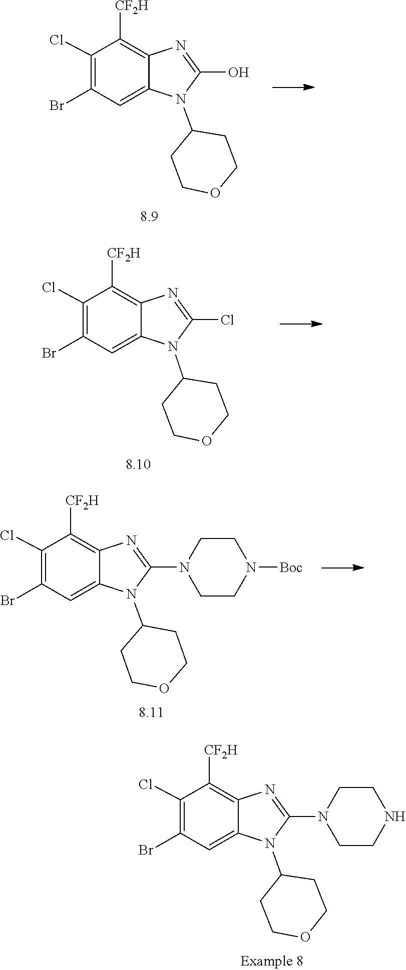 [SMILES: Oc1nc2c(C(F)F)c(Cl)c(Br)cc2n1C1CCOCC1 ; FC(F)c1c(Cl)c(Br)cc2c1nc(Cl)n2C1CCOCC1 ; CC(C)(C)OC(=O)N1CCN(c2nc3c(C(F)F)c(Cl)c(Br)cc3n2C2CCOCC2)CC1 ; FC(F)c1c(Cl)c(Br)cc2c1nc(N1CCNCC1)n2C1CCOCC1]

Step 1: To a solution of 2-fluoro-6-nitrobenzaldehyde (10.00 g, 59.14 mmol) in DCM (100 mL) was added DAST (39.07 mL, 295.68 mmol) dropwise at 0° C. After addition, the mixture was stirred at 50° C. for 2 hrs under N₂. After cooling to room temperature, the mixture was quenched with ice water (200 mL) and extracted with DCM (100 mL×2). The combined organic layers were washed with brine (200 mL), dried over Na₂SO₄, and concentrated to dryness under reduced pressure to afford a residue, which was purified by column chromatography (SiO₂, PE:EtOAc=10:1 to 5:1) to afford the desired product.

Step 2: To a solution of 8.1 (11.00 g, 57.56 mmol) in DMSO (50 mL) were added TEA (17.50 g, 172.93 mmol) and DMB-NH₂ (19.20 g, 114.83 mmol) at 20° C. After addition, the reaction was stirred at 100° C. for 12 hrs. After cooling to room temperature, the resulting mixture was diluted with water (300 mL) and extracted with EtOAc (200 mL×2). The combined the organic layers were washed with brine (200 mL), dried over Na₂SO₄, filtered, and concentrated under reduced pressure to give a residue, which was purified by silica gel chromatography eluted with (PE:EtOAc=50:1 to 10:1) to afford the desired product. LC-MS (ESI+): Rt=2.380 min; m/z 337.1 [M−H]⁻.

Step 3: A solution of 8.2 (12.00 g, 35.47 mmol) in 2,2,2-trifluoroacetic acid (20 mL, 0.15 mmol) was stirred at 25° C. for 2 hrs under N₂. The reaction mixture was put into ice-water (200 mL) and adjusted pH to 10 with aq. NaOH while a large amount of solid precipitated, filtered, and the filter cake was washed with water (50 mL×2), dried to dryness under reduced pressure to afford the desired product. LC-MS (ESI+): Rt=1.393 min; m/z 189.2 [M+H]⁺.

Step 4: To a solution of 8.3 (6.80 g, 36.14 mmol) in ACN (30 mL) was added NCS (5.30 g, 39.70 mmol) at 20° C. After addition, the reaction mixture was stirred at 60° C. for 4 hrs under N₂. After cooling to 20° C., the resulting mixture was quenched with water (100 mL) and extracted with EtOAc (100 mL×2). The combined organic layers were washed with water (100 mL×2), brine (100 mL×2), dried over Na₂SO₄, filtered, and concentrated under reduced pressure to give a residue, which was purified by column chromatography (SiO₂, PE:EtOAc=10:1 to 1:1) to afford the desired product. LC-MS (ESI+): Rt=1.674 min; m/z 223.1 [M+H]⁺.

Step 5: To a solution of 8.4 (1.00 g, 4.49 mmol) in AcOH (2 mL) was added Br₂ (0.37 mL, 6.88 mmol) at 20° C. After addition, the reaction mixture was stirred at 80° C. for 10 hrs. After cooling to 20° C., the resulting was poured to water (20 mL) and extracted with EtOAc (50 mL×2). The combined organic layers were washed with water (50 mL×2), brine (50 mL×2), filtrate and concentrated under reduced pressure to give a residue, which was purified by column chromatography (SiO₂, PE:EtOAc=10:1 to 1:1) to afford the desired product. LC-MS (ESI+): Rt=1.549 min; m/z 299.1 [M−H]⁻.

Step 6: To a solution of 8.5 (500 mg, 1.66 mmol) in ACN (3 mL) were added Copper (I) chloride (164.5 mg, 1.66 mmol), tert-butyl nitrite (256.9 mg, 2.49 mmol) at 0° C. After addition, the mixture was stirred at 25° C. for 7 hrs. The reaction mixture was quenched with water (30 mL), extracted with EtOAc (20 mL×2). The combined organic layers were washed with brine (30 mL×2), dried over Na₂SO₄, filtered, and concentrated under reduced pressure to give a residue, which was purified by prep-TLC (SiO₂, PE:EtOAc=3:1) to afford the desired product. ¹H NMR (400 MHz, CDCl₃) δ 7.98 (s, 1H), 6.99 (t, J=52.0 Hz, 1H).

Step 7: To a solution of 8.6 (100.0 mg, 0.31 mmol) and DIEA (0.050 mL, 0.31 mmol) in NMP (3 mL) was added tetrahydropyran-4-amine (47.30 mg, 0.47 mmol) at 20° C. After addition, the mixture was stirred at 80° C. for 4 hrs under N₂. After cooling to 20° C., the resulting mixture was quenched with water (10 mL) and extracted with EtOAc (10 mL×3). The combined organic layers were washed with water (20 mL×2), brine (20 mL), dried over Na₂SO₄, filtered, and concentrated under reduced pressure to give a residue, which was purified by prep-TLC (SiO₂, PE:EtOAc=5:1) to afford the desired product. LC-MS (ESI+): Rt=2.223 min; m/z 384.9 [M+H]⁺.

Step 8: To a solution of 8.7 (100.0 mg, 0.26 mmol) and NH₄Cl (138.7 mg, 2.59 mmol) in EtOH (5 mL) and H₂O (1 mL) was added Fe powder (144.7 mg, 2.59 mmol) at 0° C. After addition, the reaction mixture was stirred at 50° C. for 2 hrs. After cooling to 20° C., the resulting mixture was filtered and the filtrate was concentrated to afford the desired product. LC-MS (ESI+): Rt=2.099 min; m/z 355.0 [M+H]⁺.

Step 9: To a solution of 8.8 (77.0 mg, 0.22 mmol) and TEA (0.03 mL, 0.22 mmol) in DCM (5 mL) was added triphosgene (64.2 mg, 0.22 mmol) at 0° C. After addition, the reaction mixture was stirred at 0° C. for 10 mins. The resulting mixture was poured into water (30 mL) and extracted with DCM (30 mL×2). The combined organic layers were washed with brine (50 mL×2), dried over Na₂SO₄, filtered, and concentrated to dryness under reduced pressure to afford the desired product. LC-MS (ESI+): Rt=1.683 min; m/z 381.0 [M+H]⁺.

Step 10: A solution of 8.9 (50.0 mg, 0.13 mmol) in POCl₃ (0.5 mL) was stirred at 80° C. for 10 hrs. After cooling to 20° C., the resulting mixture was diluted with EtOAc (10 mL) and water (20 mL), extracted with EtOAc (20 mL×3). The combined organic layers were washed with brine (30 mL), dried over Na₂SO₄, filtered, and concentrated under reduced pressure to afford the desired product. LC-MS (ESI+): Rt=2.140 min; m/z 398.9 [M+H]⁺.

Step 11: To a solution of 8.10 (20.0 mg, 0.05 mmol) and DIEA (0.04 mL, 0.25 mmol) in NMP (2 mL) was added 2-methylpropan-2-yl piperazine-1-carboxylate (27.9 mg, 0.15 mmol) at 20° C. After addition, the reaction mixture was stirred at 130° C. for 4 hrs under N₂. After cooling to 20° C., the resulting mixture was quenched with water (15 mL) and extracted with EtOAc (20 mL×3). The combined organic layers were washed with brine (20 mL×2), dried over Na₂SO₄, filtered, and concentrated to dryness under reduced pressure to afford a residue, which was purified by prep-TLC (SiO₂, PE:EtOAc=1:1) to afford the desired product. LC-MS (ESI+): Rt=3.256 min; m/z 549.0 [M+H]⁺.

Step 12: To a solution of 8.11 (15.0 mg, 0.027 mmol) in EtOAc (2 mL) was added HCl/dioxane (4 m, 2 mL) at 0° C. After addition, the mixture was stirred at 20° C. for 4 hrs. The mixture was concentrated to dryness under reduced pressure to afford a residue, which was purified by pre-HPLC (Column: YMC-ACTUS Prep OBD C18 20× 250 mm×5 μm; Mobile phase: from 40% to 65% ACN in water [0.1% NH₃·H₂O]; Wavelength: 220 nm; Flow rate: 20 mL/min.) to afford the desired product. LC-MS (ESI+): Rt=1.474 min; m/z 449.0 [M+H]⁺. ¹H NMR (400 MHz, methanol-d₄) δ 8.14 (s, 1H), 7.50 (t, J=54.0 Hz, 1H), 4.56-4.53 (m, 1H), 4.21-4.12 (m, 2H), 3.70-3.62 (m, 2H), 3.61-3.54 (m, 4H), 3.53-3.49 (m, 4H), 2.56-2.46 (m, 2H), 1.93-1.81 (m 2H).

### Example 9

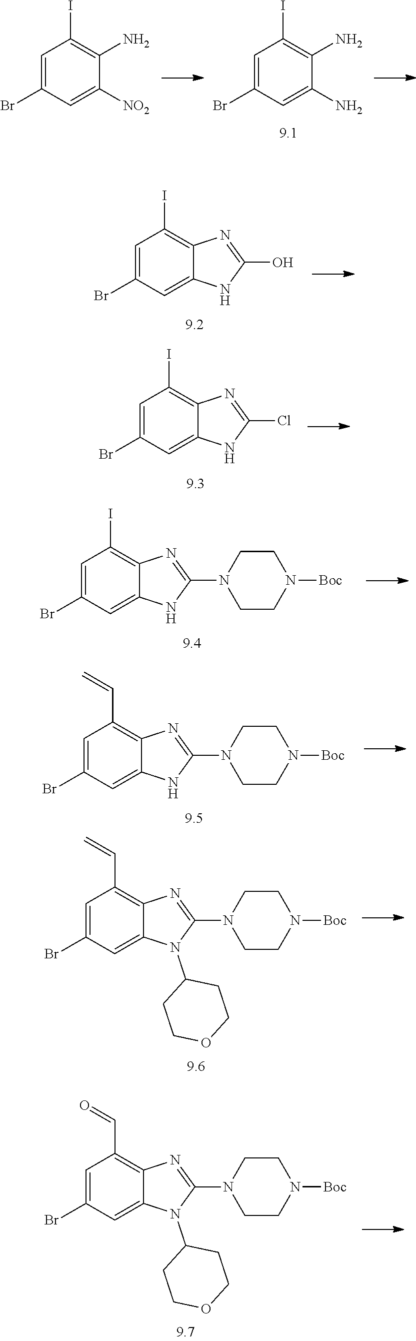

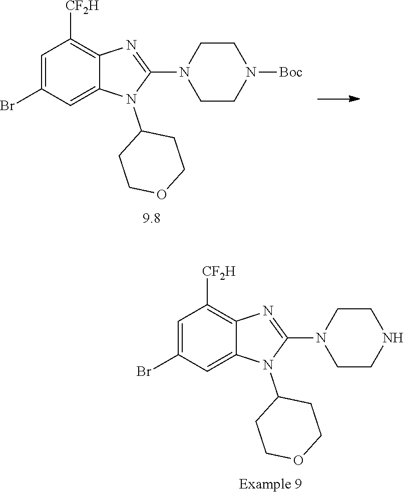 [SMILES: CC(C)(C)OC(=O)N1CCN(c2nc3c(C(F)F)cc(Br)cc3n2C2CCOCC2)CC1 ; FC(F)c1cc(Br)cc2c1nc(N1CCNCC1)n2C1CCOCC1]

Step 1: To a solution of 4-bromo-2-iodo-6-nitroaniline (4.00 g, 11.67 mmol) in EtOH (100 mL) and water (20 mL) were added Fe (6.50 g, 116.65 mmol) and NH₄Cl (6.20 g, 116.65 mmol) at 0° C. After addition, the mixture was stirred at 50° C. for 2 hrs. After cooling to room temperature, the resulting suspension was filtered. The filter cake was washed with EtOH (100 mL×3). The combined filtrates were concentrated to dryness under reduced pressure to give a residue, which was adjusted pH to 8 with saturated sodium bicarbonate (200 mL) and then extracted with EtOAc (100 mL×3). The combined organic layers were concentrated to dryness under reduced pressure to afford the desired product. LC-MS (ESI+): Rt=1.693 min; m/z 312.9 [M+H]⁺.

Step 2: To a solution of 9.1 (3.50 g, 11.18 mmol) in toluene (50 mL) was added CDI (2.70 g, 16.78 mmol) at 20° C. After addition, the mixture was stirred at 80° C. for 2 hrs. After cooling to room temperature, water (200 mL) was added and the mixture was filtered. The filter cake was washed with EtOH (100 mL×3) and concentrated to dryness under reduced pressure to afford the desired product. LC-MS (ESI+): Rt=1.106 min; m/z 338.8 [M+H]⁺.

Step 3: A solution of 9.2 (1.00 g, 2.96 mmol) in POCl₃ (10 mL) was stirred at 100° C. for 12 hrs. After cooling to room temperature, the resulting mixture was concentrated to dryness under reduced pressure (remove POCl₃) to give a residue, which was diluted with water (50 mL) and extracted with EtOAc (30 mL×2). Then the combined organic layers were washed with brine (50 mL×2), dried over Na₂SO₄, filtered, and concentrated under reduced pressure to afford the desired product. LC-MS (ESI+): Rt=1.811 min; m/z 356.8 [M+H]⁺.

Step 4: To a solution of 9.3 (900.0 mg, 2.52 mmol) in NMP (5 mL) were added 2-methylpropan-2-yl piperazine-1-carboxylate (938.30 mg, 5.04 mmol) and DIEA (1626.7 mg, 12.59 mmol) at 20° C. After addition, the mixture was stirred at 130° C. for 4 hrs under N₂. After cooling to 20° C., the resulting mixture was diluted with water (50 mL) and then extracted with EtOAc (30 mL×2). The combined organic layers were washed with water (50 mL×2), brine (50 mL×2), dried over Na₂SO₄ and concentrated under reduced pressure to give a residue, which was purified by column chromatography (SiO₂, PE:EA=6:1 to 3:1) to afford the desired product. LC-MS (ESI+): Rt=1.888 min; m/z 506.8 [M+H]⁺.

Step 5: To a solution of 9.4 (1.00 g, 1.98 mmol), Na₂CO₃ (600.0 mg, 5.92 mmol) and potassiumtrifluoro (vinyl) borate (800.0 mg, 5.92 mmol) in dioxane (20 mL) and H₂O (4 mL) was added Pd(dppf)Cl₂ (100.0 mg, 0.20 mmol) under N₂. After addition, the mixture was purged with N₂ for 2 mins and then stirred at 80° C. for 12 hrs under N₂. After cooling to room temperature, the resulting mixture was poured to water (40 mL) and extracted with EtOAc (30 mL×3). The combined organic layers were washed with brine (50 mL×2), dried over Na₂SO₄, filtered, and concentrated under reduced pressure to give a residue, which was purified by column chromatography (SiO₂, PE:EtOAc:THF=10:1 to 5:1) to afford the desired product. LC-MS (ESI+): Rt=1.151 min; m/z 406.8 [M+H]⁺.

Step 6: To a mixture of 9.5 (400.0 mg, 0.98 mmol), Cs₂CO₃ (1920.4 mg, 5.89 mmol) and TBAI (108.8 mg, 0.30 mmol) in NMP (5 mL) was added intermediate 5 (1061.8 mg, 5.89 mmol) at 20° C. After addition, the mixture was stirred at 130° C. for 4 hrs. After cooling to 20° C., the resulting mixture was diluted with water (20 mL) and extracted with EtOAc (20 mL×2). The combined organic layers were washed with H₂O (20 mL×3), brine (20 mL×2), dried over Na₂SO₄, filtered, and concentrated under reduced pressure to give a residue, which was purified by prep-TLC (SiO₂, PE:EtOAc=1:1) to afford the desired product. LC-MS (ESI+): Rt=2.638 min; m/z 491.1 [M+H]⁺.

Step 7: To a solution of 9.6 (450.0 mg, 0.92 mmol) in THF (5 mL) and water (5 mL) were added NaIO₄ (585.2 mg, 2.75 mmol) and K₂OsO₄·2H₂O (28.1 mg, 0.092 mmol) at 0° C. After addition, the reaction was stirred at 20° C. for 2 hrs. To the resulting mixture was added water (30 mL) and extracted with EtOAc (30 mL×2). The combined organic layers were washed with brine (30 mL×2), dried over Na₂SO₄, filtered, and concentrated under reduced pressure to afford the crude product (400.0 mg, 0.81 mmol, crude) as yellow oil, which was used for next step without further purification.

Step 8: To a solution of 9.7 (400.0 mg, 0.81 mmol) in DCM (10 mL) was added DAST (0.54 mL, 4.05 mmol) at 0° C. After addition, the mixture was stirred at 50° C. for 2 hrs under N₂. After cooling to room temperature, the resulting mixture was diluted with water (30 mL) and extracted with DCM (30 mL×2). The combined organic layers were washed with brine (30 mL×2), dried over Na₂SO₄, filtered, and concentrated under reduced pressure to give a residue, which was purified by prep-TLC (SiO₂, PE:EtOAc=2:1) to afford the desired product. LC-MS (ESI+): Rt=2.555 min; m/z 514.8 [M+H]⁺.

Step 9: To a solution of 9.8 (50.0 mg, 0.097 mmol) in EtOAc (2 mL) was added HCl/1,4-dioxane (4M, 1 mL) at 0° C. After addition, the mixture was stirred at 20° C. for 1 hr. The reaction mixture was concentrated under reduced pressure to give a residue, which was purified by prep-HPLC (Column: Waters Xbridge Prep OBD C18 20× 250 mm×10 μm; Mobile phase: from 20% to 50% ACN in water [0.1% NH₃·H₂O]; Wavelength: 220 nm; Flow rate: 20 mL/min.) to afford the desired product. LC-MS (ESI+): Rt=0.907 min; m/z 415.1 [M+H]⁺. ¹H NMR (400 MHz, methanol-d₄) δ 7.88 (s, 1H), 7.51 (s, 1H), 7.29 (t, J=56 Hz, 1H), 4.59-4.48 (m, 1H), 4.20-4.13 (m, 2H), 3.67-3.59 (m, 2H), 3.31-3.24 (m, 4H), 3.11-3.05 (m, 4H), 2.59-2.46 (m, 2H), 1.89-1.79 (m, 2H).

### Example 10

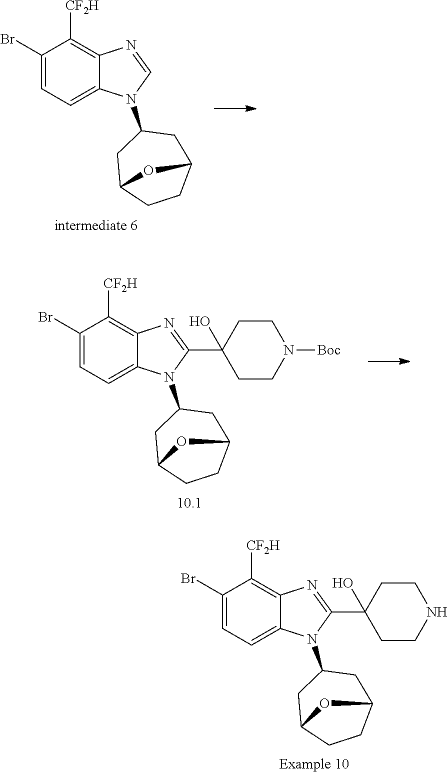 [SMILES: FC(F)c1c(Br)ccc2c1ncn2[C@@H]1C[C@H]2CC[C@@H](C1)O2 ; CC(C)(C)OC(=O)N1CCC(O)(c2nc3c(C(F)F)c(Br)ccc3n2[C@@H]2C[C@H]3CC[C@@H](C2)O3)CC1 ; OC1(c2nc3c(C(F)F)c(Br)ccc3n2[C@@H]2C[C@H]3CC[C@@H](C2)O3)CCNCC1]

Step 1: To a solution of intermediate 6 (50.0 mg, 0.14 mmol) in THF (5 mL) was added LDA (0.14 mL, 2M) dropwise at −78° C. over 5 min. After addition, the mixture was stirred at −78° C. for 1 hr, and then tert-butyl 4-oxopiperidine-1-carboxylate (55.8 mg, 0.28 mmol) in THF (1 mL) was added dropwise at −78° C. The resulting mixture was stirred at −78° C. for 2 hrs under N₂. The resulting mixture was quenched with water (30 mL) and extracted with EtOAc (30 mL×2). The organic layers were washed with water (50 mL×2), brine (50 mL×2), dried over Na₂SO₄, filtered, and concentrated under reduced pressure to give a residue, which was purified by prep-TLC (SiO₂, PE:EtOAc=1:1) to afford the desired product. LC-MS (ESI+): Rt=3.191 min; m/z 555.8 [M+H]⁺.

Step 2: To a solution of 10.1 (25.0 mg, 0.045 mmol) in DCM (3 mL) was added TFA (1 mL) at 0° C. After addition, the mixture was stirred at 20° C. for 2 hrs under N₂. The mixture was concentrated to dryness under reduced pressure to afford a residue, which was purified by prep-HPLC (Column: waters Xbridge Prep OBD C18 20× 250 mm×5 μm; Mobile layer: from 40% to 70% ACN in water [0.1% NH₄HCO₃]; Wavelength: 220 nm; Flow rate: 20 mL/min) to afford desired product. LC-MS (ESI+): Rt=0.597 min; m/z 455.8 [M+H]⁺. ¹H NMR (400 MHz, DMSO-d₆) δ 7.69-7.40 (m, 3H), 5.93-5.80 (m, 2H), 4.53 (s, 2H), 3.36-3.30 (m, 1H), 3.03-2.97 (m, 2H), 2.90-2.87 (m, 2H), 2.43-2.40 (m, 2H), 2.21-2.15 (m, 2H), 1.97-1.88 (m, 4H), 1.81-1.76 (m, 4H).

### Example 14

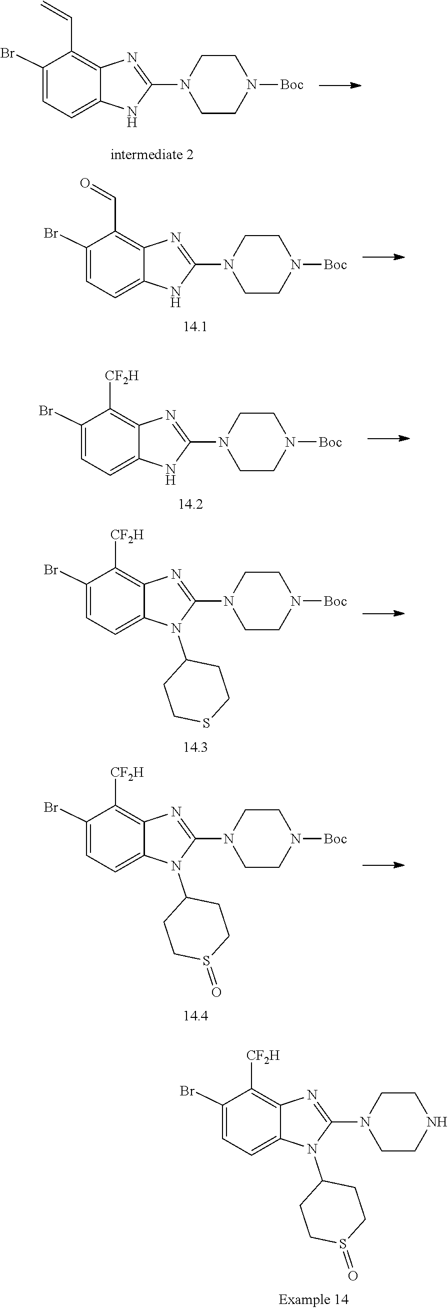 [SMILES: C=Cc1c(Br)ccc2[nH]c(N3CCN(C(=O)OC(C)(C)C)CC3)nc12 ; CC(C)(C)OC(=O)N1CCN(c2nc3c(C=O)c(Br)ccc3[nH]2)CC1 ; CC(C)(C)OC(=O)N1CCN(c2nc3c(C(F)F)c(Br)ccc3[nH]2)CC1 ; CC(C)(C)OC(=O)N1CCN(c2nc3c(C(F)F)c(Br)ccc3n2C2CCSCC2)CC1 ; CC(C)(C)OC(=O)N1CCN(c2nc3c(C(F)F)c(Br)ccc3n2C2CCS(=O)CC2)CC1 ; O=S1CCC(n2c(N3CCNCC3)nc3c(C(F)F)c(Br)ccc32)CC1]

Step 1: To a solution of intermediate 2 (500.0 mg, 1.23 mmol) in THF (10 mL) and water (10 mL) was added NaIO₄ (785.0 mg, 3.69 mmol) and K₂OsO₄·2H₂O (31.2 mg, 0.12 mmol) at 0° C. After addition, the reaction was stirred at 20° C. for 2 hrs. The reaction solution was poured into water (50 mL) and extracted with EtOAc (30 mL×3). The combined organic layers were washed with brine (50 mL×2), dried over Na₂SO₄, filtered, and concentrated under reduced pressure to afford the desired product. LC-MS (ESI+): Rt=1.876 min; m/z 408.9 [M+H]⁺.

Step 2: To a solution of 14.1 (500.0 mg, 1.22 mmol) in DCM (5 mL) was added DAST (0.81 mL, 6.11 mmol) dropwise at 0° C. After addition, the mixture was stirred at 50° C. for 2 hrs under N₂. After cooling to 20° C., the resulting solution was poured into ice water (100 mL) and extracted with DCM (50 mL×3). The combined organic layers were washed with water (50 mL×2), brine (50 mL×2), dried over Na₂SO₄, filtered, and concentrated under reduced pressure to give a residue, which was purified by column chromatography (SiO₂, PE:EtOAc=1:1) to afford the desired product. LC-MS (ESI+): Rt=1.958 min; m/z 430.9 [M+H]⁺.

Step 3: To a solution of 14.2 (380.0 mg, 0.88 mmol), Cs₂CO₃ (861.1 mg, 2.64 mmol) and Tetrabutylammonium iodide (32.5 mg, 0.09 mmol) in NMP (5 mL) was added thian-4-yl methanesulfonate (518.8 mg, 2.64 mmol). After addition, the mixture was stirred at 130° C. for 4 hrs. After cooling to 20° C., the reaction solution was poured into water (100 mL) and extracted with EtOAc (30 mL×3). The combined organic layers were concentrated to dryness under reduced pressure to give a residue, which was purified by column chromatography (SiO₂, PE:EtOAc=1:1) to afford desired product. LC-MS (ESI+): Rt=3.542 min; m/z 531.1 [M+H]⁺.

Step 4: To a solution of 14.3 (50.0 mg, 0.09 mmol) in DCM (1 mL) and ACN (3 mL) was added Oxone (0.06 mL, 0.12 mmol) at 0° C. After addition, the reaction was stirred at 0° C. for 3 hrs. The resulting mixture was diluted with saturated aq. Na₂SO₃ (5 mL). After that, the reaction solution was poured into water (20 mL) and extracted with EtOAc (30 mL×2). The combined organic layers were washed with water (20 mL×2), brine (20 mL×2), filtrated and concentrated under reduced pressure to give a residue, which was purified by column chromatography (SiO₂, DCM:MeOH=10:1) to afford the desired product. LC-MS (ESI+): Rt=2.466 min; m/z 547.0 [M+H]⁺.

Step 5: To a solution of 14.4 (15.0 mg, 0.03 mmol) in EtOAc (1 mL) was added HCl/1,4-dioxane (4M, 2 mL). After addition, the mixture was stirred at 0° C. for 1 hr. The resulting mixture was concentrated to dryness under reduced pressure, which was purified by prep-HPLC (Column: Waters Xbridge Prep OBD C18 20× 250 mm×10 μm; Mobile layer: from 15% to 50% ACN in water [0.1% NH₄OH]; Wavelength: 220 nm; Flow rate: 20 mL/min) to afford the desired product. LC-MS (ESI+): Rt=1.484 min; m/z 447.0 [M+H]⁺. ¹H NMR (400 MHz, methanol-d₄) δ 7.74-7.72 (d, J=8.0 Hz, 1H), 7.55-7.28 (m, 2H), 4.60-4.52 (m, 1H), 3.42-3.39 (m, 4H), 3.30-3.21 (m, 6H), 3.14-3.07 (m, 2H), 3.02-2.92 (m, 2H), 1.99-1.92 (m, 2H).

### Example 19

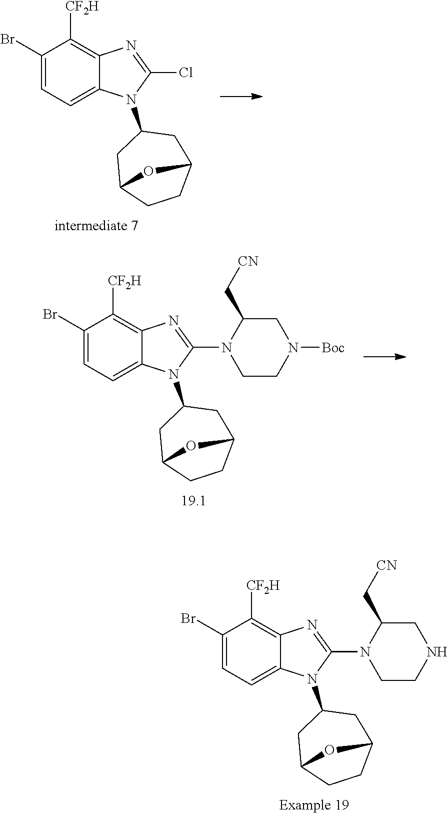 [SMILES: FC(F)c1c(Br)ccc2c1nc(Cl)n2[C@@H]1C[C@H]2CC[C@@H](C1)O2 ; CC(C)(C)OC(=O)N1CCN(c2nc3c(C(F)F)c(Br)ccc3n2[C@@H]2C[C@H]3CC[C@@H](C2)O3)[C@@H](CC#N)C1 ; N#CC[C@H]1CNCCN1c1nc2c(C(F)F)c(Br)ccc2n1[C@@H]1C[C@H]2CC[C@@H](C1)O2]

Step 1: To a solution of intermediate 7 (500.0 mg, 1.28 mmol) in NMP (1 mL) were added DIEA (1.1 mL, 6.38 mmol) and tert-butyl(S)-3-(cyanomethyl) piperazine-1-carboxylate (1.44 g, 6.38 mmol) at 20° C. After addition, the mixture was stirred at 150° C. for 12 hrs under N₂. After cooling to 20° C., the reaction solution was poured into water (30 mL) and extracted with EtOAc (30 mL×3). The combined organic layers were washed with water (100 mL×2), brine (100 mL×2), filtered and concentrated under reduced pressure to give a residue, which was purified by column chromatography (SiO₂, PE:EtOAc=5:1 to 3:1) to afford the desired product. LC-MS (ESI+): Rt=3.307 min; m/z 580.0 [M+H]⁺.

Step 2: To a solution of 19.1 (1.10 g, 1.90 mmol) in EtOAc (30 mL) was added HCl/1,4-dioxane (20 mL, 4M) at 0° C. After addition, the mixture was stirred at 20° C. for 1 hr under N₂. The mixture was concentrated to dryness under reduced pressure to afford a residue, which was purified by prep-HPLC (Column: waters Xbridge Prep OBD C18 20×250 mm×5 μm; Mobile layer: from 35% to 65% ACN in water [0.1% NH₄OH]; Wavelength: 220 nm; Flow rate: 15 mL/min) to afford the desired product. LC-MS (ESI+): Rt=1.371 min; m/z 479.8 [M+H]⁺. ¹H NMR (400 MHz, methanol-d₄) δ 7.68-7.66 (d, J=8.0 Hz, 1H), 7.51-7.49 (d, J=8.0 Hz, 1H), 7.43 (t, J=52.0 Hz, 1H), 5.12-5.03 (m, 1H), 4.66-4.63 (m, 1H), 4.57-4.56 (m, 1H), 3.82-3.78 (m, 1H), 3.28-3.24 (m, 2H), 3.11-2.95 (m, 5H), 2.80-2.75 (m, 1H), 2.64-2.48 (m, 2H), 2.15-2.05 (m, 3H), 1.99-1.91 (m, 3H).

The following compounds were prepared using a synthesis method similar to that described in Example 19.

| | LC-MS (ESI+) | |
|---|---|---|
| Example | [M + H]⁺ | ¹H NMR (400 MHz) |
| 15 | 441.9 | methanol-d₄ δ 8.36 (s, 1H), 7.49-7.23 (t, J = 52.0 Hz, 1H), 4.69-4.62 |
| | | (m, 1H), 4.61-4.56 (m, 2H), 3.38-3.36 (m, 4H), 3.10-3.08 (m, 4H), |
| | | 3.03-2.95 (m, 2H), 2.13-2.09 (m, 2H), 1.96-1.90 (m, 2H), 1.78- |
| | | 1.72 (m, 2H). |
| 16 | 484.9 | methanol-d₄ δ 7.58-7.30 (m, 3H), 4.81-4.72 (m, 1H), 4.61 (s, 2H), |
| | | 3.79-3.76 (t, J = 6.0 Hz, 2H), 3.37-3.32 (m, 4H), 2.84-2.82 (m, |
| | | 4H), 2.72-2.69 (m, 2H), 2.61-2.53 (m, 2H), 2.15-2.12 (m, 2H), |
| | | 1.97-1.92 (m, 2H), 1.82-1.78 (m, 2H) |
| 17 | 499.0 | methanol-d₄ δ 7.58-7.28 (m, 3H), 4.77-4.71 (m, 1H), 4.59 (s, 2H), |
| | | 3.66-3.63 (t, J = 6.0 Hz, 2H), 3.40-3.37 (m, 7H), 3.13-2.88 (m, |
| | | 6H), 2.59-2.51 (m, 2H), 2.13-2.09 (m, 2H), 1.96-1.90 (m, 2H), |
| | | 1.81-1.76 (m, 2H) |
| 24 | 410.9 | methanol-d₄ δ 7.73-7.31 (m, 3H), 5.16-5.07 (m, 1H), 4.64-4.59 (m, |
| | | 2H), 3.53-3.48 (m, 1H), 3.19-3.07 (m, 4H), 3.03-2.97 (m, 1H), |
| | | 2.71-2.51 (m, 3H), 2.17-2.13 (m, 2H), 1.99-1.95 (m, 2H), 1.88- |
| | | 1.84 (m, 1H), 1.70-1.65 (m, 1H), 1.01-1.00 (d, J = 4.0 Hz, 3H). |
| 25 | 397.9 | methanol-d₄ δ 8.30 (s, 1H), 7.53-7.25 (t, J = 52.0 Hz, 1H), 4.70-4.61 |
| | | (m, 1H), 4.60-4.56 (m, 2H), 3.56-3.54 (m, 4H), 3.38-3.32 (m, 4H), |
| | | 3.04-2.97 (m, 2H), 2.13-2.10 (m, 2H), 1.99-1.91 (m, 2H), 1.79- |
| | | 1.75 (m, 2H). |
| 28 | 455.0 | methanol-d₄ δ 7.67-7.65 (d, J = 8.0 Hz, 1H), 7.51-7.49 (d, J = 8.0 Hz, |
| | | 1H), 7.58-7.31 (t, J = 54.0 Hz, 1H), 5.15-5.06 (m, 1H), 4.65-4.59 |
| | | (m, 2H), 3.58-3.53 (m, 1H), 3.23-3.12 (m, 4H), 3.10-3.03 (m, 1H), |
| | | 2.80-2.75 (m, 1H), 2.64-2.51 (m, 2H), 2.16-2.13 (m, 2H), 2.00- |
| | | 1.94 (m, 2H), 1.87-1.83 (m, 1H), 1.71-1.66 (m, 1H), 1.03-1.01 (d, |
| | | J = 8.0 Hz, 3H). |
| 30 | 510.9 | DMSO-d₆ δ 7.46 (t, J = 54.0 Hz, 1H), 7.52-7.50 (d, J = 8.0 Hz, 1H), |
| | | 7.41-7.39 (d, J = 8.0 Hz, 1H), 5.29-7.27 (d, J = 8.0 Hz, 1H), 4.56- |
| | | 4.51 (m, 3H), 4.20-4.15 (m, 1H), 3.54-3.51 (m, 2H), 3.42-3.33 (m, |
| | | 2H), 3.02-2.95 (m, 2H), 2.73-2.70 (m, 2H), 2.41-2.37 (m, 2H), |
| | | 2.24-2.20 (m, 1H), 2.00-1.97 (m, 2H), 1.84-1.72 (m, 6H), 1.43- |
| | | 1.34 (m, 2H). |
| 34 | 483.9 | methanol-d₄ δ 7.54-7.27 (m, 3H), 4.77-4.68 (m, 1H), 4.60 (s, 2H), |
| | | 3.69-3.66 (t, J = 12.0 Hz, 2H), 3.48-3.44 (m, 2H), 3.10-3.03 (m, |
| | | 2H), 2.59-2.51 (m, 2H), 2.13-2.10 (m, 2H), 1.95-1.91 (m, 3H), |
| | | 1.80-1.75 (m, 3H), 1.62-1.59 (m, 2H), 1.47-1.43 (m, 2H), 1.29 (m, |
| | | 1H). |
| 37 | 466.7 | methanol-d₄ δ 7.58-7.29 (m, 3H), 4.83-4.81 (m, 1H), 4.63-4.61 (m, |
| | | 2H), 3.64-3.61 (m, 2H), 3.35-3.32 (m, 2H), 3.19-3.15 (m, 2H), |
| | | 2.61-2.54 (m, 2H), 2.18-2.15 (m, 2H), 2.10-2.05 (m, 2H), 2.04- |
| | | 1.94 (m, 4H), 1.83-1.78 (m, 2H). |
| 39 | 497.8 | methanol-d₄ δ 7.56-7.29 (m, 3H), 4.81-4.70 (m, 1H), 4.60 (s, 2H), |
| | | 4.35-4.28 (m, 1H), 3.24-3.21 (m, 2H), 3.19-3.15 (m, 2H), 2.60- |
| | | 2.53 (m, 2H), 2.40-2.35 (m, 2H), 2.15-2.12 (m, 2H), 1.95-1.90 (m, |
| | | 2H), 1.84-1.76 (m, 8H). |
| 40 | 496.8 | methanol-d₄ δ 7.59-7.30 (m, 3H), 4.82-4.75 (m, 1H), 4.62 (s, 2H), |
| | | 4.50-4.45 (m, 1H), 3.57-3.53 (m, 2H), 3.35-3.28 (m, 1H), 3.29- |
| | | 3.25 (m, 1H), 3.18-3.15 (m, 1H), 2.99-2.93 (m, 1H), 2.76-2.69 (m, |
| | | 1H), 2.65-2.54 (m, 3H), 2.29-2.25 (m, 1H), 2.15-2.13 (m, 2H), |
| | | 1.96-1.94 (m, 2H), 1.86-1.82 (m, 4H). |
| 41 | 468.8 | methanol-d₄ δ 7.58-7.31 (m, 3H), 4.82-4.76 (m, 1H), 4.64-4.59 (m, |
| | | 2H), 3.59-3.55 (m, 1H), 3.22-3.11 (m, 4H), 2.97-2.93 (m, 1H), |
| | | 2.65-2.49 (m, 2H), 2.16-2.12 (m, 2H), 1.96-1.83 (m, 3H), 1.71- |
| | | 1.66 (m, 1H), 1.28-1.23 (m, 6H). |
| 42 | 468.8 | methanol-d₄ δ 7.66-7.64 (d, J = 8.0 Hz, 1H), 7.49-7.47 (d, J = 8.0 |
| | | Hz, 1H), 7.42 (t, J = 54.0 Hz, 1H), 5.23-5.14 (m, 1H), 4.63-4.57 (m, |
| | | 2H), 3.43-3.38 (m, 1H), 3.12-3.08 (m, 1H), 3.05-2.98 (m, 2H), |
| | | 2.74-2.49 (m, 4H), 2.15-2.10 (m, 2H), 1.95-1.90 (m, 2H), 1.85- |
| | | 1.80 (m, 1H), 1.66-1.62 (m, 1H), 1.12 (d, J = 8.0 Hz, 3H), 0.90 (d, J = |
| | | 8.0 Hz, 3H). |
| 47 | 466.8 | methanol-d₄ δ 7.40 (t, J = 54.0 Hz, 1H), 7.47-7.45 (d, J = 8.0 Hz, |
| | | 1H), 7.33-7.31 (d, J = 8.0 Hz, 1H), 4.68-4.62 (m, 1H), 4.57 (s, 2H), |
| | | 3.97-3.93 (m, 1H), 3.72-3.68 (m, 2H), 3.58-3.54 (m, 1H), 3.18- |
| | | 3.08 (m, 2H), 2.60-2.52 (m, 2H), 2.34-2.31 (m, 1H), 2.11-2.01 (m, |
| | | 3H), 1.99-1.90 (m, 4H), 1.81-1.74 (m, 2H). |
| 44 | 441.8 | DMSO-d₆ δ 7.48 (t, J = 54.0 Hz, 1H), 7.56-7.54 (d, J = 8.0 Hz, 1H), |
| | | 7.43-7.43 (d, J = 8.0 Hz, 1H), 4.67-4.58 (m, 1H), 4.54-4.51 (m, |
| | | 2H), 3.83-3.81 (m, 4H), 3.23-3.21 (m, 4H), 2.43-2.36 (m, 2H), |
| | | 2.00-1.97 (m, 2H), 1.87-1.84 (m, 2H), 1.79-1.75 (m, 2H). |
| 51 | 497.0 | DMSO-d₆ δ 7.59-7.32 (m, 4H), 6.80 (s, 1H), 4.54-4.48 (m, 3H), |
| | | 3.44-3.41 (m, 2H), 3.00-2.93 (m, 2H), 2.41-2.33 (m, 2H), 2.09- |
| | | 2.07 (m, 2H), 2.00-1.89 (m, 3H), 1.84-1.72 (m, 6H), 1.42-1.32 (m, |
| | | 2H). |

### Example 26

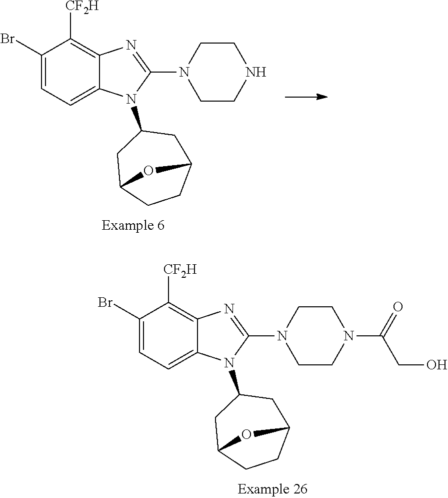 [SMILES: FC(F)c1c(Br)ccc2c1nc(N1CCNCC1)n2[C@@H]1C[C@H]2CC[C@@H](C1)O2 ; O=C(CO)N1CCN(c2nc3c(C(F)F)c(Br)ccc3n2[C@@H]2C[C@H]3CC[C@@H](C2)O3)CC1]

To a solution of example 6 (20.0 mg, 0.045 mmol) and 2-hydroxyacetic acid (3.8 mg, 0.05 mmol) in DMF (1 mL) were added HATU (20.7 mg, 0.05 mmol) and TEA (22.9 mg, 0.23 mmol) at 0° C. After addition, the mixture was stirred at 20° C. for 4 hrs under N₂. The reaction mixture was poured into water (50 mL) and extracted with EtOAc (30 mL×3). The combined organic layers were washed with water (100 mL×2), brine (100 mL×2), filtered and concentrated under reduced pressure to give a residue, which was purified by prep-HPLC (Column: waters Xbridge Prep OBD C18 20×250 mm×5 um; Mobile layer: from 30% to 70% ACN in water [0.1% NH₄OH]; Wavelength: 220 nm; Flow rate: 20 mL/min) to afford the desired product. LC-MS (ESI+): Rt=2.341 min; m/z 498.7 [M+H]⁺. ¹H NMR (400 MHz, methanol-d₄) δ 7.61-7.29 (m, 3H), 4.83-4.79 (m, 1H), 4.62 (s, 2H), 4.33 (s, 2H), 3.90-3.80 (m, 2H), 3.75-3.62 (m, 2H), 3.35-3.31 (m, 4H), 2.63-2.55 (m, 2H), 2.15-2.12 (m, 2H), 2.00-1.97 (m, 2H), 1.86-1.81 (m, 2H).

### Example 29

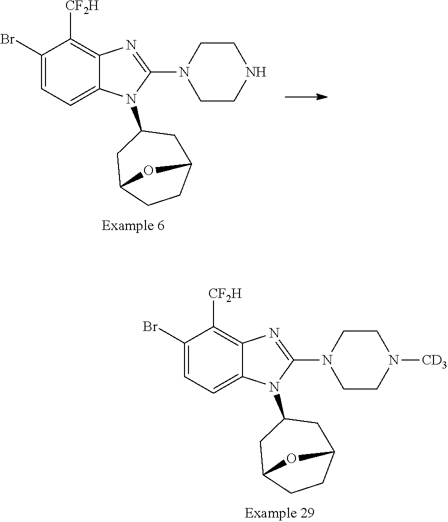 [SMILES: FC(F)c1c(Br)ccc2c1nc(N1CCNCC1)n2[C@@H]1C[C@H]2CC[C@@H](C1)O2 ; [2H]C([2H])([2H])N1CCN(c2nc3c(C(F)F)c(Br)ccc3n2[C@@H]2C[C@H]3CC[C@@H](C2)O3)CC1]

To a solution of example 6 (20.0 mg, 0.14 mmol) in DMF (2 mL) was added NaH (12.8 mg, 0.11 mmol, 60%) at 0° C. After addition, the mixture was stirred at 0° C. for 10 min, and then methyl-d₃ 4-methylbenzenesulfonate (25.8 mg, 0.045 mmol) was added at 0° C., the mixture was stirred at 20° C. for 4.5 hrs under N₂. The resulting solution was quenched by water (50 mL) and extracted with EtOAc (30 mL×3). The combined organic layers were washed with water (100 mL×2), brine (100 mL×2), filtered and concentrated under reduced pressure to give a residue, which was purified by prep-HPLC (Column: waters Xbridge Prep OBD C18 20×250 mm×5 μm; Mobile layer: from 35% to 65% ACN in water [0.1% NH₄HCO₃]; Wavelength: 220 nm; Flow rate: 15 mL/min) to afford the desired product. LC-MS (ESI+): Rt=2.374 min; m/z 457.9 [M+H]⁺. ¹H NMR (400 MHz, methanol-d₄) 7.58-7.30 (m, 3H), 4.81-4.72 (m, 1H), 4.63-4.60 (m, 2H), 3.36-3.32 (m, 4H), 2.72-2.70 (m, 4H), 2.61-2.53 (m, 2H), 2.15-2.12 (m, 2H), 1.97-1.91 (m, 2H), 1.83-1.78 (m, 2H).

### Example 31

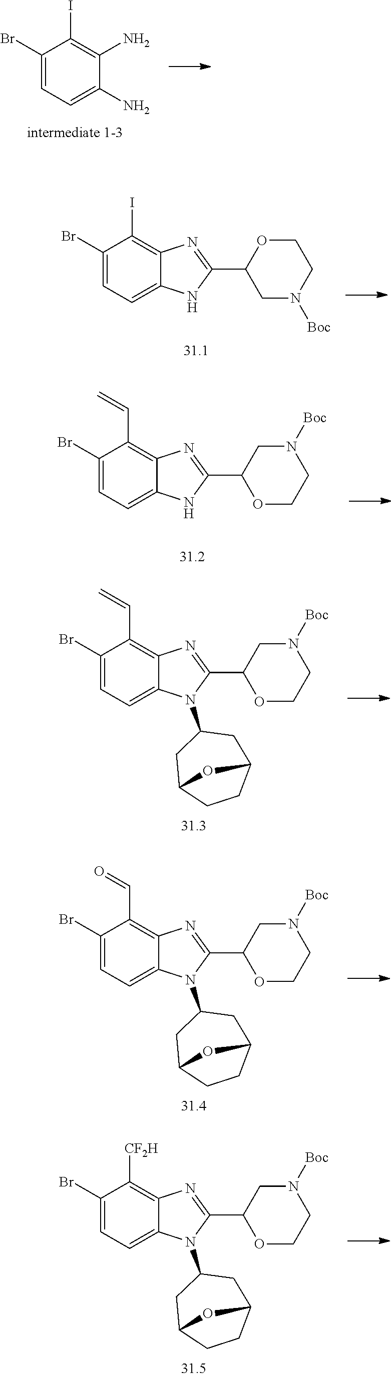 [SMILES: Nc1ccc(Br)c(I)c1N ; CC(C)(C)OC(=O)N1CCOC(c2nc3c(I)c(Br)ccc3[nH]2)C1 ; C=Cc1c(Br)ccc2[nH]c(C3CN(C(=O)OC(C)(C)C)CCO3)nc12 ; C=Cc1c(Br)ccc2c1nc(C1CN(C(=O)OC(C)(C)C)CCO1)n2[C@@H]1C[C@H]2CC[C@@H](C1)O2 ; CC(C)(C)OC(=O)N1CCOC(c2nc3c(C=O)c(Br)ccc3n2[C@@H]2C[C@H]3CC[C@@H](C2)O3)C1 ; CC(C)(C)OC(=O)N1CCOC(c2nc3c(C(F)F)c(Br)ccc3n2[C@@H]2C[C@H]3CC[C@@H](C2)O3)C1]

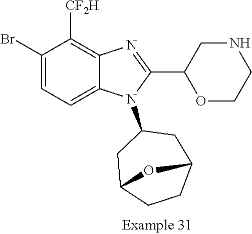 [SMILES: FC(F)c1c(Br)ccc2c1nc(C1CNCCO1)n2[C@@H]1C[C@H]2CC[C@@H](C1)O2]

Step 1: To a solution of intermediate 1-3 (7.60 g, 24.29 mmol) in methanol (100 mL) were added 2-methylpropan-2-yl-2-formyl-1,4-oxazinane-4-carboxylate (10.45 g, 48.57 mmol) and AcOH (6.95 mL, 121.45 mmol) dropwise at 0° C. After addition. The reaction was stirred at 25° C. for 12 hrs. The reaction solution was concentrated under reduced pressure to give a residue, which was poured into water (150 mL) and extracted with EtOAc (100 mL×3), washed with water (100 mL×2), brine (100 mL×2), dried over Na₂SO₄, filtered and concentrated under reduced pressure afford a residue, which was purified by column chromatography (SiO₂, PE:EtOAc=10:1 to 5:1) to afford the desired product. LC-MS (ESI+): Rt=1.923 min; m/z 507.7 [M+H]⁺.

Step 2: To a solution of 31.1 (1.50 g, 2.95 mmol) in dioxane (30 mL) and water (6 mL) were added Na₂CO₃ (685.2 mg, 6.46 mmol), potassium vinyltrifluoroborate (346.5 mg, 2.59 mmol) and Pd (dppf) C₁₂ (157.7 mg, 0.22 mmol) at 20° C. After addition, the mixture was purged with N₂ for 3 mins and then stirred at 80° C. for 12 hrs under. After cooling to 20° C., the reaction solution was poured into water (30 mL) and extracted with EtOAc (30 mL×3), washed with water (60 mL), brine (60 mL), dried over Na₂SO₄, filtered, and concentrated under reduced pressure to give a residue, which was purified by column chromatography (SiO₂, PE:EtOAc=15:1 to 10:1) to afford the desired product. LC-MS (ESI+): Rt=1.938 min; m/z 407.9 [M+H]⁺.

Step 3: To a solution of 31.2 (700.0 mg, 1.71 mmol), Cs₂CO₃ (2.79 g, 8.57 mmol) and TBAI (126.8 mg, 0.34 mmol) in NMP (20 mL) was added intermediate 4 (1.77 g, 8.57 mmol) at 20 C°. After addition, the mixture was stirred at 135° C. for 12 hrs under N₂. After cooling to 20° C., the reaction solution was poured into water (20 mL) and extracted with EtOAc (20 mL×3). The combined organic layers were washed with water (20 mL×2), brine (20 mL×2), filtrate and concentrated under reduced pressure to give a residue which was purified by prep-TLC (PE:EtOAc=3:2) to afford the desired product. LC-MS (ESI+): Rt=3.589 min; m/z 518.2 [M+H]⁺.

Step 4: To a solution of 31.3 (300.0 mg, 0.58 mmol) in THF (10 mL) and H₂O (10 mL) were added NaIO₄ (371.3 mg, 1.74 mmol) and K₂OsO₄·2H₂O (9.3 mg, 0.058 mmol) at 0° C. After addition, the mixture was stirred at 25° C. for 2 hrs under N₂. The reaction solution was poured into water (20 mL) and extracted with EtOAc (20 mL×3). The combined organic layers were washed with water (30 mL×2), brine (30 mL), dried over Na₂SO₄, filtered, and concentrated under reduced pressure to afford the desired product. LC-MS (ESI+): Rt=2.799 min; m/z 519.9 [M+H]⁺.

Step 5: To a solution of 31.4 (300.0 mg, 0.58 mmol) in DCM (5 mL) was added DAST (309.8 mg, 1.92 mmol) dropwise at 0° C. After addition, the mixture was stirred at 50° C. for 2 hrs under N₂. After cooling to 20° C., the reaction solution was poured into ice water (10 mL) and extracted with DCM (10 mL×2). The combined organic layers were washed with brine (10 mL×2), dried over Na₂SO₄, filtered, and concentrated under reduced pressure to give a residue which was purified by prep-TLC (SiO₂, PE:EtOAc=2:1) to afford the desired product LC-MS (ESI+): Rt=3.001 min; m/z 541.8 [M+H]⁺.

Step 6: To a solution of 31.5 (150.0 mg, 0.28 mmol) in EtOAc (2 mL) was added HCl/dioxane (4 mL, 16.00 mmol) dropwise at 0° C. After addition, the mixture was stirred at 25° C. for 2 hr. The mixture was concentrated to dryness under reduced pressure to afford a residue, which was purified by prep-HPLC (SHIMADZULC-20AP, Column: WelchXB-C18, 20×250 mm, 10 μm. Mobile Layer A: 0.1% NH₄HCO₃, B: ACN; flow rate: 20 mL/min) to afford desired product. LC-MS (ESI+): Rt=0.526 min; m/z 441.8 [M+H]⁺. ¹H NMR (400 MHz, methanol-d₄) δ 7.77-7.75 (d, J=8.0 Hz, 1H), 7.49 (t, J=56.0 Hz, 1H), 7.58-7.55 (d, J=12.0 Hz, 1H), 5.30-5.22 (m, 1H), 5.00-4.90 (m, 1H), 4.63 (s, 2H), 3.95-3.90 (m, 1H), 3.86-3.80 (m, 1H), 3.41-3.34 (m, 1H), 3.30-3.29 (m, 1H), 2.98-2.95 (m, 2H), 2.65-2.55 (m, 2H), 2.16-2.09 (m, 2H), 2.04-1.97 (m, 2H), 1.91-1.84 (m, 2H).

The following compounds were prepared using a synthesis method similar to that described in Example 31.

| | LC-MS (ESI+) | |
|---|---|---|
| Example | [M + H]⁺ | ¹H NMR (400 MHz) |
| 32 | 457.9 | methanol-d₄ δ 7.77-7.75 (d, J = 8.0 Hz, 1H), 7.48 (t, J = 56.0 Hz, |
| | | 1H), 7.57-7.54 (d, J = 12.0 Hz, 1H), 5.31-5.25 (m, 1H), 5.14-5.11 |
| | | (m, 1H), 4.63 (s, 2H), 4.09-4.03 (m, 1H), 3.93-3.87 (m, 1H), 3.58- |
| | | 3.44 (m, 2H), 3.16-3.13 (m, 1H), 3.04-2.99 (m, 1H), 2.65-2.56 (m, |
| | | 2H), 2.15-2.09 (m, 2H), 2.05-1.98 (m, 4H), 1.92-1.86 (m, 2H). |
| 33 | 475.9 | methanol-d₄ δ 7.84-7.82 (d, J = 8.0 Hz, 1H), 7.62-7.35 (m, 2H), |
| | | 5.02-4.94 (m, 1H), 4.61(s, 2H), 4.48-4.44 (m, 1H), 4.06-3.98 (m, |
| | | 1H), 3.89-3.79 (m, 2H), 3.45-3.40 (m, 1H), 2.76-2.45 (m, 4H), |
| | | 2.21-2.18 (m, 1H), 2.11-2.08 (m, 2H), 2.02-1.90 (m, 2H), 1.86- |
| | | 1.82 (m, 1H). |
| 35 | 439.9 | methanol-d₄ δ 8.02-7.99 (d, J = 12.0 Hz, 1H), 7.79-7.76 (d, J = 12.0 |
| | | Hz, 1H), 7.51 (t, J = 52.0 Hz, 1H), 5.14-5.05 (m, 1H), 4.67-4.56 (m, |
| | | 2H), 3.96-3.89 (m, 1H), 3.67-3.61 (m, 2H), 3.45-3.37 (m, 2H), |
| | | 2.71-2.63 (m, 2H), 2.45-2.27 (m, 4H), 2.22-2.12 (m, 4H), 2.04- |
| | | 1.99 (m, 2H). |
| 43 | 465.9 | methanol-d₄ δ 7.70-7.68 (d, J = 8.0 Hz, 1H), 7.63-7.36 (m, 2H), 4.90- |
| | | 4.81 (m, 1H), 4.51-4.63 (m, 2H), 3.73-3.69 (m, 2H), 3.60-3.54 |
| | | (m, 1H), 2.65-2.58 (m, 2H), 2.25-1.83 (m, 14H). |

### Example 36

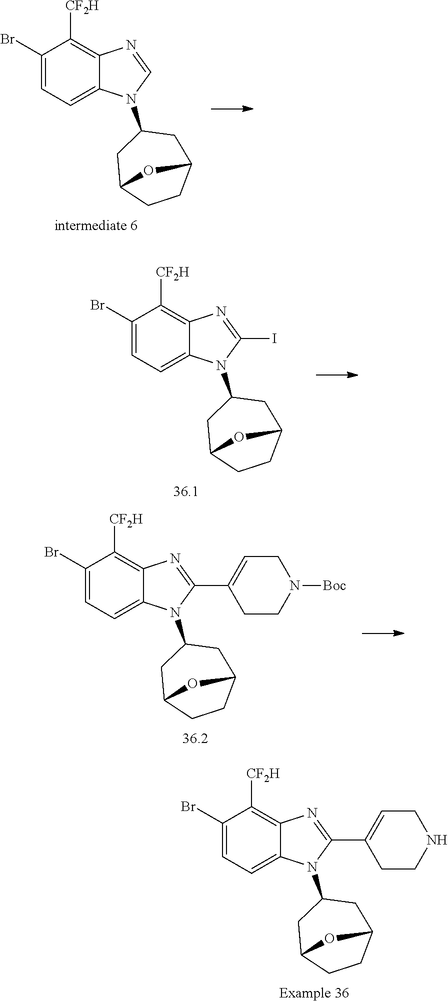 [SMILES: FC(F)c1c(Br)ccc2c1ncn2[C@@H]1C[C@H]2CC[C@@H](C1)O2 ; FC(F)c1c(Br)ccc2c1nc(I)n2[C@@H]1C[C@H]2CC[C@@H](C1)O2 ; CC(C)(C)OC(=O)N1CC=C(c2nc3c(C(F)F)c(Br)ccc3n2[C@@H]2C[C@H]3CC[C@@H](C2)O3)CC1 ; FC(F)c1c(Br)ccc2c1nc(C1=CCNCC1)n2[C@@H]1C[C@H]2CC[C@@H](C1)O2]

Step 1: To a solution of intermediate 6 (425.0 mg, 1.19 mmol) in THF (10 mL) was added LDA (1.78 mL, 2M) dropwise at −78° C. over 5 min. After addition, the mixture was stirred at −78° C. for 1 hr, and then I₂ (603.9 mg, 2.38 mmol) in THF (2 mL) was added dropwise at −78° C. The resulting mixture was stirred at −78° C. for 1 hr under N₂. The resulting mixture was quenched with water (30 mL) and extracted with EtOAc (30 mL×2). The combined organic layers were washed with brine (50 mL×2), dried over Na₂SO₄, filtered, and concentrated under reduced pressure to give a residue, which was purified by prep-TLC (SiO₂, PE:EtOAc=1:1) to afford the desired product. LC-MS (ESI+): Rt=2.807 min; m/z 482.7 [M+H]⁺.

Step 2: To a solution of 36.1 (250.0 mg, 0.52 mmol), Na₂CO₃ (164.6 mg, 1.55 mmol) and tert-butyl 4-(4,4,5,5-tetramethyl-1,3,2-dioxaborolan-2-yl)-3,6-dihydropyridine-1 (2H)-carboxylate (192.0 mg, 0.62 mmol) in 1,4-dioxane (10 mL) and water (2 mL) was added Pd(dppf)Cl₂ (37.9 mg, 0.052 mmol) at 20° C. After addition, the mixture was purged with N₂ for 2 mins and then stirred at 70° C. for 4 hrs under N₂. After cooling to 20° C., the reaction solution was poured into water (50 mL) and extracted with EtOAc (30 mL×3). The combined organic layers were washed with water (50 mL×2), brine (50 mL×2), dried over Na₂SO₄, filtered, and concentrated under reduced pressure to give a residue, which was purified by column chromatography (SiO₂, PE:EtOAc=10:1 to 2:1) to afford the desired product. LC-MS (ESI+): Rt=2.655 min; m/z 537.5 [M+H]⁺.

Step 3: To a solution of 36.2 (325.0 mg, 0.60 mmol) in EtOAc (5 mL) was added HCl/1,4-dioxane (4 mL, 4M) dropwise at 0° C. After addition, the mixture was stirred at 20° C. for 2 hrs under N₂. The mixture was concentrated to dryness under reduced pressure to afford a residue, which was purified by prep-HPLC (Column: waters Xbridge Prep OBD C18 20× 250 mm×5 μm; Mobile layer: from 20% to 70% ACN in water [0.1% NH₄OH]; Wavelength: 220 nm; Flow rate: 15 mL/min) to afford the desired product. LC-MS (ESI+): Rt=1.359 min; m/z 437.9 [M+H]⁺. ¹H NMR (400 MHz, DMSO-d₆) δ 7.74-7.41 (m, 3H), 6.22 (m, 1H), 4.94-4.85 (m, 1H), 4.56-4.52-6.22 (m, 2H), 3.47-3.45 (m, 2H), 3.31-3.10 (m, 1H), 2.98-2.95 (t, J=6.0 Hz, 2H), 2.50-2.28 (m, 4H), 1.99-1.96 (m, 2H), 1.83-1.78 (m, 4H).

The following compounds were prepared using a synthesis method similar to that described in Example 36.

| | LC-MS (ESI+) | |
|---|---|---|
| Example | [M + H]⁺ | ¹H NMR (400 MHz) |
| 46 | 424.9 | methanol-d₄ δ 8.17 (s, 2H), 7.83-7.81 (d, J = 8.0 Hz, 1H), 7.59-7.56 |
| | | (d, J = 12.0 Hz, 1H), 7.47 (t, J = 54.0 Hz, 1H), 5.11-5.02 (m, 1H), |
| | | 4.63-4.60 (m, 2H), 2.73-2.66 (m, 2H), 2.11-2.04 (m, 2H), 1.94- |
| | | 1.89 (m, 2H), 1.86-1.80 (m, 2H). |
| 49 | 423.2 | DMSO-d₆ δ 13.59 (s, 1H), 8.01 (s, 1H), 7.81-7.79 (d, J = 8.0 Hz, 1H), |
| | | 7.62 (t, J = 54.0 Hz, 1H), 7.58-7.56 (d, J = 8.0 Hz, 1H), 7.01 (s, 1H), |
| | | 6.39-6.34 (m, 1H), 4.56 (s, 2H), 3.35-3.15 (m, 2H), 2.00-1.97 (m, |
| | | 2H), 1.92-1.82 (m, 4H). |

### Example 38

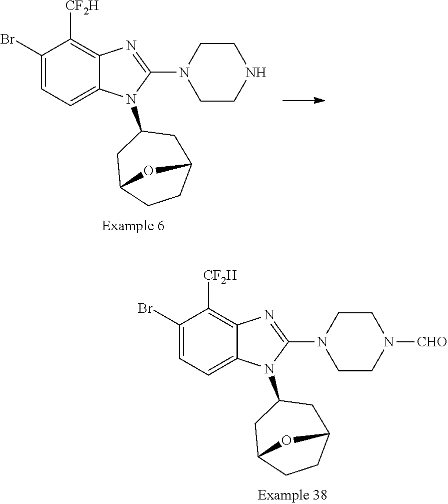 [SMILES: FC(F)c1c(Br)ccc2c1nc(N1CCNCC1)n2[C@@H]1C[C@H]2CC[C@@H](C1)O2 ; O=CN1CCN(c2nc3c(C(F)F)c(Br)ccc3n2[C@@H]2C[C@H]3CC[C@@H](C2)O3)CC1]

To a solution of example 6 (100.0 mg, 0.23 mmol) in ethyl formate (5 mL) at 20° C. After addition, the mixture was stirred at 50° C. for 12 hrs under N₂. After cooling to 20° C., the reaction solution was poured into water (50 mL) and extracted with EtOAc (30 mL×3). The combined organic layers were washed with water (50 mL×2), brine (50 mL×2), dried over Na₂SO₄, filtered and concentrated under reduced pressure to give a residue, which was purified by prep-HPLC (Column: waters Xbridge Prep OBD C18 20×250 mm×5 μm; Mobile layer: from 15% to 70% ACN in water [0.1% NH₄HCO₃]; Wavelength: 220 nm; Flow rate: 15 mL/min) to afford desired product. LC-MS (ESI+): Rt=2.090 min; m/z 469.0 [M+H]⁺. ¹H NMR (400 MHz, DMSO-d₆) δ 8.12 (s, 1H), 7.61-7.34 (m, 3H), 4.65-4.59 (m, 1H), 4.53 (s, 2H), 3.65-3.60 (m, 4H), 3.28-3.25 (m, 2H), 3.20-3.18 (m, 2H), 2.43-2.37 (m, 2H), 1.99-1.78 (m, 6H).

### Example 45

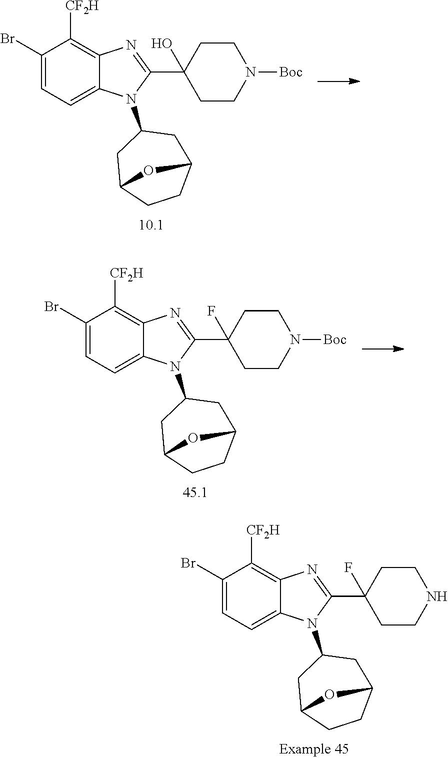 [SMILES: CC(C)(C)OC(=O)N1CCC(O)(c2nc3c(C(F)F)c(Br)ccc3n2[C@@H]2C[C@H]3CC[C@@H](C2)O3)CC1 ; CC(C)(C)OC(=O)N1CCC(F)(c2nc3c(C(F)F)c(Br)ccc3n2[C@@H]2C[C@H]3CC[C@@H](C2)O3)CC1 ; FC(F)c1c(Br)ccc2c1nc(C1(F)CCNCC1)n2[C@@H]1C[C@H]2CC[C@@H](C1)O2]

Step 1: To a solution of 10.1 (160.0 mg, 0.29 mmol) in DCM (5 mL) were added DAST (0.2 mL, 1.44 mmol) at 0° C. After addition, the mixture was stirred at 50° C. for 30 min under N₂. After cooling to 20° C., the reaction solution was poured into water (30 mL) and extracted with EtOAc (30 mL×3), the combined organic layers were washed with water (60 mL) and brine (60 mL), dried over Na₂SO₄, filtered and concentrated under reduced pressure to give a residue, which was purified by prep-TLC (SiO₂,PE:EtOAc=1:1) to afford the desired product. LC-MS (ESI+): Rt=2.336 min; m/z 579.8 [M+Na]⁺.

Step 2: To a solution of 45.1 (75.0 mg, 0.13 mmol) in EtOAc (1 mL) was added HCl/dioxane (2 mL, 4 M) at 0° C. After addition, the mixture was stirred at 25° C. for 2 hrs. The reaction solution was concentrated to dryness under reduced pressure to afford a residue, which was purified by prep-HPLC (SHIMADZU LC-20AP, Column: YMC Triart C18, 20× 250 mm, 5 μm; Mobile Layer A: 0.1% NH₄HCO₃, B: ACN; flow rate: 15 mL/min; gradient: 25-55%.) to afford the desired product. LC-MS (ESI+): Rt=0.505 min; m/z 457.9 [M+H]⁺. ¹H NMR (400 MHz, methanol-d₄) δ 7.77-7.75 (d, J=8.0 Hz, 1H), 7.57-7.54 (d, J=12.0 Hz, 1H), 7.47 (t, J=54.0 Hz, 1H), 5.39-5.32 (m, 1H), 4.60 (s, 2H), 3.13-3.08 (m, 4H), 2.67-2.60 (m, 2H), 2.52-2.37 (m, 2H), 2.28-2.22 (m, 2H), 2.12-2.09 (m, 2H), 1.94-1.83 (m, 4H).

The following compound was prepared using a synthesis method similar to that described in Example 45.

| | LC-MS (ESI+) | |
|---|---|---|
| Example | [M + H]⁺ | ¹H NMR (400 MHz) |
| 50 | 444.0 | methanol-d₄ δ 7.79-7.77 (d, J = 8.0 Hz, 1H), 7.59-7.57 (d, J = 8.0 |
| | | Hz, 1H), 7.47 (t, J = 54.0 Hz, 1H), 5.23-5.14 (m, 1H), 4.62 (s, 2H), |
| | | 3.95-3.85 (m, 1H), 3.66-3.57 (m, 1H), 3.38-3.33 (m, 1H), 3.23- |
| | | 3.18 (m, 1H), 2.80-2.69 (m, 1H), 2.68-2.60 (m, 3H), 2.14-2.10 (m, |
| | | 2H), 1.95-1.84 (m, 4H). |

### Example 48

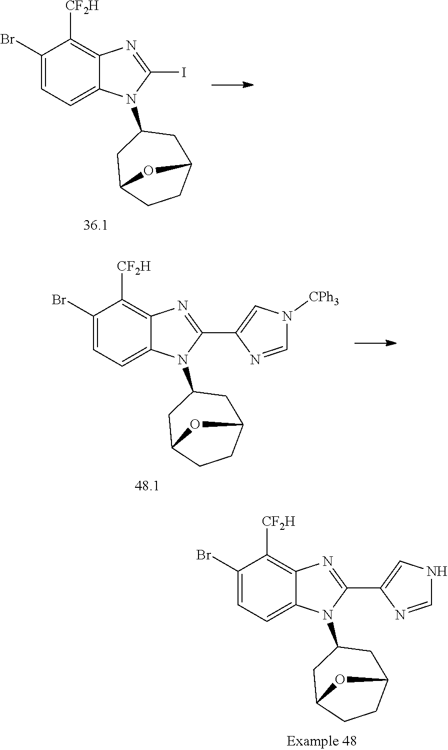

Step 1: To a solution of 36.1 (43.0 mg, 0.09 mmol) in dioxane (1 ml) were added lithium chloride (5.6 mg, 0.13 mmol) and 4-(tributylstannyl)-1-trityl-1H-imidazole (63.8 mg, 0.11 mmol) and Pd₂(dba)₃ (8.1 mg, 8.86 μmol) at 20° C. After addition, the mixture was purged with N₂ for 3 mins and then stirred at 100° C. for 12 hrs under N₂. After cooling to 20° C., the reaction solution was poured into water (5 mL) and extracted with EtOAc (5 mL×3). The combined organic layers were washed with brine (10 mL×2), dried over Na₂SO₄, filtered, and concentrated under reduced pressure to give a residue which was purified by prep-TLC (SiO₂, PE:EtOAc=1:1) to afford the desired product. LC-MS (ESI+): Rt=4.027 min; m/z 665.1 [M+H]⁺.

Step 2: To a solution of 48.1 (40.0 mg, 0.06 mmol) in DCM (2 mL) was added TFA (463 μL, 6.0 mmol) at 0° C. After addition, the mixture was stirred at 25° C. for 2 hrs. The reaction mixture was concentrated under reduced pressure to give a residue which was purified by pre-HPLC (SHIMADZU LC-20AP, Column: YMC Triart C18, 20×50 mm, 5 μm; Mobile Phase A: 0.1% NH₄OH, B: ACN; flow rate: 15 mL/min; gradient: 25-55%.) to afford desired product. LC-MS (ESI+): Rt=1.573 min; m/z 422.9 [M+H]⁺. ¹H NMR (400 MHz, DMSO-d₆) δ 12.78 (s, 1H), 7.98-7.97 (m, 2H), 7.57 (t, J=54.0 Hz, 1H), 7.73-7.71 (d, J=8.0 Hz, 1H), 7.50-7.48 (d, J=8.0 Hz, 1H), 6.64-6.54 (m, 1H), 4.53 (s, 2H), 2.01-1.88 (m, 4H), 1.83-1.78 (m, 2H), 1.30-1.15 (m, 2H).

### BIOLOGICAL EXAMPLES

### Example A1: CDK8 Kinase Assay

Compound IC₅₀ were determined against CDK8/cyclinC.

Compound serial dilution is performed by Echo, and the final concentrations vary from 1 μM to 0.017 nM. This was filled by the addition of 5 μL/well of Enzyme & antibodies mixture to the assay plate containing the compound. The plate was centrifuged at 1000 rpm about 15 seconds, and incubate 15 minutes at 23° C. Then 5 L/well of tracer solution was added to initiate the reaction. Next the plate was centrifuged at 1000 rpm for about 15 seconds and a film seal was used to cover the assay plate. This was followed by incubation for 90 min at 23° C.

The assay plate was analyzed on Envision.

Representative results are shown in Table 3.

TABLE 3

| | CDK8/ |
|---|---|
| Example | CycC, IC₅₀ |
| 1 | A |
| 2 | A |
| 3 | B |
| 4 | A |
| 5 | B |
| 6 | A |
| 7 | A |
| 8 | A |
| 9 | B |
| 10 | B |
| 11 | B |
| 12 | B |
| 13 | B |
| 14 | B |
| 15 | B |
| 16 | A |
| 17 | B |
| 18 | A |
| 19 | A |
| 20 | B |
| 21 | A |
| 22 | A |
| 23 | A |
| 24 | A |
| 25 | B |
| 26 | A |
| 27 | A |
| 28 | A |
| 29 | A |
| 30 | A |
| 31 | B |
| 32 | B |
| 33 | A |
| 34 | A |
| 35 | A |
| 36 | B |
| 37 | A |
| 38 | A |
| 39 | B |
| 40 | B |
| 41 | A |
| 42 | B |
| 43 | C |
| 44 | A |
| 45 | A |
| 46 | A |
| 47 | C |
| 48 | B |
| 49 | B |
| 50 | B |
| 51 | A |

$$A : 0 < {IC}_{50} \leq 10. ( nM )$$

$$B : 10. < I {C}_{50} \leq 100 ( nM )$$

$$C : 100 < I {C}_{50} \leq 1000 ( nM )$$

$$D : 1000 < I {C}_{50} ( nM )$$

### Example A2: In Vivo PK

The animals (male, n=3 per group) were administered a single oral dose. Blood samples were collected in K2EDTA-coated tubes by 0.25, 0.5, 1, 2, 4, 6, 8 and 24 h post-dose and were centrifuged to obtain plasma. The plasma concentration was determined by protein precipitation and LC-MS/MS detection. Examples showed good plasma exposure.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound of Formula (Ia), or a pharmaceutically acceptable salt or stereoisomer thereof: 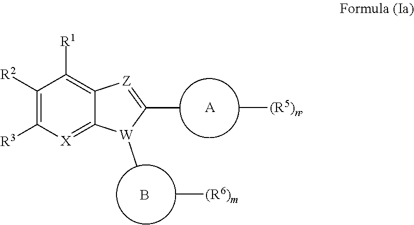
wherein:
R¹ is halogen, —CN, —OH, —OR^{a}, —NR^{c}R^{d}, —C(═O)R^{a}, —C(═O)OR⁵, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
R² is fluoro, chloro, or bromo and R³ is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or
R³ is fluoro, chloro, or bromo and R² is hydrogen, fluoro, chloro, iodo, —CN, —OH, —OR^{a}, —S(═O)R^{a}, —S(═O)₂R^{a}, —S(═O)₂NR^{c}R^{d}, —S(═O)(═NR^{b})R^{b}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
X is N or CR⁴;
R⁴ is hydrogen, halogen, —CN, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
W is —N—;
Z is —N— or —CR^{Z1}—;
R^{Z1} is hydrogen, halogen, —SF₅, —CN, —NO₂, —OH, —OR^{a}, —NR^{c}R^{d}, —C(═O)R^{a}, —C(═O)OR^{b}, —C(═O)NR^{c}R^{d}, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
Ring A is cycloalkyl, heterocycloalkyl, aryl, or heteroaryl;
each R⁵ is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NRDC(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{5a};
or two R⁵ on the same atom are taken together to form an oxo;
each R^{5a} is independently halogen, —CN, —NO₂, —OH, —OR^{a}, —OC(═O)R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{5a} on the same atom are taken together to form an oxo;
n is 0, 1, 2, 3, 4, 5, or 6;
Ring B is cycloalkyl, heterocycloalkyl, aryl, or heteroaryl;
each R⁶ is independently halogen, —CN, —NO₂, —OH, —OR^{a}, —OC(═O) R^{a}, —OC(═O)OR^{b}, —OC(═O)NR^{c}R^{d}, —SF₅, —SH, —SR^{a}, —S(═O)R^{a}, —S(═O)₂R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NR^{b}C(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R^{6a};
or two R⁶ on the same atom are taken together to form an oxo;
or two R⁶ on the same atom are taken together to form ═NH;
each R^{6a} is independently halogen, —CN, —NO₂, —OH, —OR^{a}, —OC(═O) R^{a}, —OC(═O)OR^{b}, —OC(═O)NR^{c}R^{d}, —SF₅, —SH, —SR^{a}, —S(═O)R^{a}, —S(═O) ¿R^{a}, —S(═O)₂NR^{c}R^{d}, —S(═O)(═NR^{b})R^{b}, —NR^{c}R^{d}, —NR^{b}C(═O)NR^{c}R^{d}, —NR^{b}C(═O)R^{a}, —NRDC(═O)OR^{b}, —NR^{b}S(═O)₂R^{a}, —N═S(═O)(R^{b})₂, —C(═O)R^{a}, —C(═O)OR^{b}, —C(═O)NR^{c}R^{d}, —P(═O)(R^{b})₂, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one or more R;
or two R^{6a} on the same atom are taken together to form an oxo;
m is 0, 1, 2, 3, 4, 5, or 6;
each R^{a} is independently C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
each R^{b} is independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
R^{c} and R^{d} are each independently hydrogen, C₁-C₆alkyl, C₁-C₆haloalkyl, C₁-C₆hydroxyalkyl, C₁-C₆aminoalkyl, C₁-C₆heteroalkyl, C₂-C₆alkenyl, C₂-C₆alkynyl, -L-cycloalkyl, -L-heterocycloalkyl, -L-aryl, or -L-heteroaryl, wherein each alkyl, alkenyl, alkynyl, cycloalkyl, heterocycloalkyl, aryl, and heteroaryl is independently optionally substituted with one or more R;
or R^{c} and R^{d} are taken together with the atom to which they are attached to form a heterocycloalkyl optionally substituted with one or more R; and
L is absent or C₁-C₃ alkylene optionally substituted with one or more R;
each R is independently halogen, —CN, —OH, —SF₅, —SH, —S(═O) C₁-C₃alkyl, —S(═O)₂C₁-C₃alkyl, —S(═O)₂NH₂, —S(═O)₂NHC₁-C₃alkyl, —S(═O)₂N(C₁-C₃alkyl)₂, —S(═O)(═NC₁-C₃alkyl)(C₁-C₃alkyl), —NH₂, —NHC₁-C₃alkyl, —N(C₁-C₃alkyl)₂, —N═S(═O)(C₁-C₃alkyl)₂, —C(═O)C₁-C₃alkyl, —C(═O)OH, —C(═O)OC₁-C₃alkyl, —C(═O)NH₂, —C(═O)NHC₁-C₃alkyl, —C(═O)N(C₁-C₃alkyl)₂, —P(═O)(C₁-C₃alkyl)₂, C₁-C₃alkyl, C₁-C₃alkoxy, C₁-C₃haloalkyl, C₁-C₃haloalkoxy, C₁-C₃hydroxyalkyl, C₁-C₃aminoalkyl, C₁-C₃heteroalkyl, or C₃-C₆cycloalkyl.

2. The compound of claim 1, or a pharmaceutically acceptable salt or stereoisomer thereof, wherein Z is —N—.

3. The compound of claim 1 or 2, or a pharmaceutically acceptable salt or stereoisomer thereof, wherein R² is bromo and R³ is hydrogen, fluoro, chloro, C₁-C₆alkyl, or C₁-C₆haloalkyl.

4. The compound of any one of claims 1-3, or a pharmaceutically acceptable salt or stereoisomer thereof, wherein R² is bromo and R³ is hydrogen.

5. The compound of claim 1 or 2, or a pharmaceutically acceptable salt or stereoisomer thereof, wherein R² is chloro and R³ is hydrogen, fluoro, chloro, C₁-C₆alkyl, or C₁-C₆haloalkyl.

6. The compound of claim 1, 2, or 5, or a pharmaceutically acceptable salt or stereoisomer thereof, wherein R² is chloro and R³ is hydrogen.

7. The compound of any one of claims 1-6, or a pharmaceutically acceptable salt or stereoisomer thereof, wherein R¹ is C₁-C₆haloalkyl.

8. The compound of any one of claims 1-7, or a pharmaceutically acceptable salt or stereoisomer thereof, wherein R¹ is CF₂H.

9. The compound of any one of claims 1-8, or a pharmaceutically acceptable salt or stereoisomer thereof, wherein X is CR⁴.

10. The compound of any one of claims 1-9, or a pharmaceutically acceptable salt or stereoisomer thereof, wherein R⁴ is hydrogen or halogen.

11. The compound of any one of claims 1-10, or a pharmaceutically acceptable salt or stereoisomer thereof, wherein Ring A is heterocycloalkyl.

12. The compound of any one of claims 1-11, or a pharmaceutically acceptable salt or stereoisomer thereof, wherein Ring A is a monocyclic 5- to 7-membered heterocycloalkyl comprising one or two heteroatoms selected from the group consisting of N and O.

13. The compound of any one of claims 1-11, or a pharmaceutically acceptable salt or stereoisomer thereof, wherein Ring A is a bicyclic 6- to 10-membered heterocycloalkyl comprising one or two heteroatoms selected from the group consisting of N and O.

14. The compound of any one of claims 1-13, or a pharmaceutically acceptable salt or stereoisomer thereof, wherein each R⁵ is independently —OH, —NR^{c}R^{d}, C₁-C₆alkyl, C₁-C₆haloalkyl, C₁-C₆hydroxyalkyl, or C₂-C₆alkynyl; wherein each alkyl and alkynyl is independently optionally substituted with one or more R^{5a}; or two R⁵ on the same atom are taken together to form an oxo.

15. The compound of any one of claims 1-14, or a pharmaceutically acceptable salt or stereoisomer thereof, wherein each R^{5a} is independently —CN or —OH.

16. The compound of any one of claims 1-15, or a pharmaceutically acceptable salt or stereoisomer thereof, wherein n is 0, 1, or 2.

17. The compound of any one of claims 1-16, or a pharmaceutically acceptable salt or stereoisomer thereof, wherein Ring B is cycloalkyl or heterocycloalkyl.

18. The compound of any one of claims 1-17, or a pharmaceutically acceptable salt or stereoisomer thereof, wherein Ring B is 5- to 7-membered cycloalkyl.

19. The compound of any one of claims 1-17, or a pharmaceutically acceptable salt or stereoisomer thereof, wherein Ring B is heterocycloalkyl.

20. The compound of any one of claim 1-17 or 19, or a pharmaceutically acceptable salt or stereoisomer thereof, wherein Ring B is a monocyclic or bicyclic 3- to 10-membered heterocycloalkyl comprising one, two, or three heteroatoms selected from the group consisting of N, O, S, and P.

21. The compound of any one of claim 1-17, 19, or 20, or a pharmaceutically acceptable salt or stereoisomer thereof, wherein Ring B is a monocyclic 5- to 7-membered heterocycloalkyl comprising one or two heteroatoms selected from the group consisting of N, O, and S.

22. The compound of any one of claim 1-17 or 19, or a pharmaceutically acceptable salt or stereoisomer thereof, wherein Ring B is a bicyclic 6- to 10-membered heterocycloalkyl comprising one or two heteroatoms selected from the group consisting of N, O, and S.

23. The compound of any one of claims 1-22, or a pharmaceutically acceptable salt or stereoisomer thereof, wherein each R⁶ is independently halogen, —CN, —OH, —OR^{a}, —NR^{c}R^{d}, C₁-C₆alkyl, or C₁-C₆haloalkyl; or two R⁶ on the same atom are taken together to form an oxo; or two R⁶ on the same atom are taken together to form ═NH.

24. The compound of any one of claims 1-23, or a pharmaceutically acceptable salt or stereoisomer thereof, wherein each R⁶ is independently —OH or C₁-C₆alkyl; or two R⁶ on the same atom are taken together to form an oxo.

25. The compound of any one of claims 1-24, or a pharmaceutically acceptable salt or stereoisomer thereof, wherein m is 0, 1, 2, 3, or 4.

26. A compound selected form a compound found in Table 1 or Table 2, or a pharmaceutically acceptable salt or stereoisomer thereof.

27. A compound of any one of claims 1-26, or a pharmaceutically acceptable salt or stereoisomer thereof wherein the compound is a CDK8 inhibitor.

28. A pharmaceutical composition comprising a compound of any one of claims 1-27, or a pharmaceutically acceptable salt or stereoisomer thereof, and at least one pharmaceutically acceptable excipient.

29. A method of inhibiting cyclic dependent kinase 8 and/or 19 (CDK8/19) activity in a subject, comprising administering to the subject a compound of any one of claims 1-27, or a pharmaceutically acceptable salt or stereoisomer thereof, or a pharmaceutical composition of claim 28.

30. A method of treating cancer in a subject in need thereof, the method comprising administering a therapeutically effective amount of a compound of any one of claims 1-27, or a pharmaceutically acceptable salt or stereoisomer thereof, or a pharmaceutical composition of claim 28.

31. The method of claim 30, wherein the cancer is leukemia, acute myeloid leukemia (AML), chronic myeloid leukemia, acute lymphoblastic leukemia (ALL), non-Hodgkin lymphoma (NHL), Hodgkin lymphoma (HL), or multiple myeloma (MM).

32. The method of claim 30 or 31, wherein the cancer is AML.

33. The method of claim 30, wherein the cancer is a solid cancer.

34. The method of claim 30 or 33, wherein the cancer is a prostate cancer, breast cancer, colon cancer, pancreatic cancer, skin cancer, ocular cancer, gastrointestinal cancer, thyroid cancer, ovarian cancer, central nervous system cancer, laryngeal cancer, cervical cancer, lymphatic system cancer, genitourinary tract cancer, bone cancer, biliary tract cancer, endometrial cancer, liver cancer, lung cancer, head and neck squamous cell carcinomas, melanoma.

35. A method of treating an auto-immune disease or disorder in a subject in need thereof, the method comprising administering a therapeutically effective amount of a compound of any one of claims 1-27, or a pharmaceutically acceptable salt or stereoisomer thereof, or a pharmaceutical composition of claim 28.
